(19) United States
(12) Patent Application Publication
(10) Pub. No.: US 2025/0109434 A1
(43) Pub. Date: Apr. 3, 2025

(54) CLEAVABLE CYCLIC LOOP NUCLEOTIDES FOR NANOPORE SEQUENCING

(71) Applicant: Illumina, Inc., San Diego, CA (US)

(72) Inventors: Hugo Brandão, Somerville, MA (US); John Moon, Rancho Santa Fe, CA (US); Jeffrey G. Mandell, La Jolla, CA (US); Erin Garcia, Lemon Grove, CA (US); Lisa Savagian, San Diego, CA (US); Eunho Noh, San Diego, CA (US); Xiangyuan Yang, Singapore (SG); Sharyuen Soh, Singapore (SG); Sadeer Salam, Singapore (SG)

(73) Assignee: Illumina, Inc., San Diego, CA (US)

(21) Appl. No.: 18/900,392

(22) Filed: Sep. 27, 2024

Related U.S. Application Data
(60) Provisional application No. 63/586,916, filed on Sep. 29, 2023.

(51) Int. Cl. C12Q 1/6869; C12Q 1/6809

(52) U.S. Cl. CPC C12Q 1/6869; C12Q 1/6809

(57) ABSTRACT

A polymer is disposed through a nanopore that encodes a polynucleotide's sequence and includes monomer units that encode nucleotides and include first and second reporter moieties and first and second arresting constructs. The first reporter moiety is translocated into the nanopore's aperture, where a first value of an electrical property of the first reporter moiety is measured while the first arresting construct pauses translocation. The second reporter moiety is translocated into the aperture, where a second value of an electrical property of the second reporter moiety is measured while the second arresting construct pauses translocation. The first value and the second value for each monomer unit is used to identify the nucleotide encoded by that monomer unit; and distinguish the nucleotide encoded by that monomer unit from the nucleotides encoded by adjacent monomer units, including by those that encode the same type of nucleotide as that monomer unit.

### CROSS-REFERENCE TO RELATED APPLICATIONS

This application claims the benefit of U.S. Provisional Patent Application No. 63/586,916, filed Sep. 29, 2023 and entitled “Cleavable Cyclic Loop Nucleotides for Nanopore Sequencing,” the entire contents of which are incorporated by reference herein.

### BACKGROUND

Some polynucleotide sequencing techniques involve performing a large number of controlled reactions on support surfaces or within predefined reaction chambers. The controlled reactions may then be observed or detected, and subsequent analysis may help identify properties of the polynucleotide involved in the reaction. Examples of such sequencing techniques include next-generation sequencing or massive parallel sequencing involving sequencing-by-ligation, sequencing-by-synthesis, reversible terminator chemistry, or pyrosequencing approaches.

Some polynucleotide sequencing techniques utilize a nanopore, which can provide a path for an ionic electrical current. For example, as the polynucleotide traverses through the nanopore, the polynucleotide influences the electrical current through the nanopore. Each passing nucleotide, or series of nucleotides, that passes through the nanopore yields a characteristic blockage current. These characteristic electrical currents of the traversing polynucleotide can be recorded to determine the sequence of the polynucleotide.

Errors may result in the readout of various polynucleotides, such as the readout of homopolymer sequences where two or more of the same nucleotide reside directly adjacent to each other in a polynucleotide. Thus, there has been a need to improve the accuracy of nanopore sequencing and synthesize polynucleotide derivatives in order to inhibit or prevent reading errors as the polynucleotide is translocated through the nanopore.

### SUMMARY

Traditionally the readhead of nanopores (e.g., the constriction region of nanopores) “senses” several bases concurrently along the sample DNA strand via an electrical current, increasing the challenge of accurate nanopore sequencing due to many permutations of signals arising from different sequences. For example, the MspA pore reads at least 4 bases as a time, giving rise to at least 4\hat{ }4=256 different signals that needs to be deconvoluted and resolved. To overcome the large possible number of signals that could be read from four nucleotide bases, derivative nucleotide bases have been developed. In this way, the original DNA nucleotides are not read in groups, but modified derivatives of DNA nucleotides are read individually. Daughter strand nucleotides may be modified to contain cyclic loops that create a covalent bridge from the nucleotide to the phosphate backbone of that nucleotide. For example, cyclic loops have been synthesized to contain a unique barcoding/reporter region that is specific to the original bases (e.g., A, T, C, or G for DNA or A, U, C, G in RNA) and a cleavable site. The daughter strand may be then “elongated” by cutting the cleavable sites. Additionally, linker groups and/or spacers may be incorporated into the cyclic loop structure in order to elongate the cleaved cyclic loop further and increase the spacing between the readout regions of the cyclic loop. In some examples, the cyclic loop modification may contain both barcoding/reporter regions and non-barcoding linker groups. In some examples, the linker group may be the barcoding/reporter region. Consequently, when sequencing the daughter strand, the nanopore can directly “read” each individual nucleotide via the reporter region of the cyclic loop associated with that nucleotide.

The cyclic loop may also contain one or more “arresting” constructs (ARC), which may operate to stop or slow down the translocation of the daughter sequence through the nanopore. With the incorporation of arresting constructs the nucleotide sequence may be advanced through the nanopore in a “ratcheted” manner. The advancement of the nucleotide sequence through the nanopore may be performed either in “auto-ratchet” mode (with stochastic and fairly unpredictable advancement of the nucleotide sequence) or in “pulsed-ratchet” mode which promotes the advancement of nucleotides (along with their arresting constructs) in fairly pre-defined time intervals. Such sequencing modes may be referred to herein as ratchet-based nanopore sequencing.

Despite these modifications, the readout of the daughter strand containing the derivative cyclic loops may have errors of up to 10%. For example, the readout of the nucleotide sequence may be inaccurate due to “deletion errors,” “mismatch errors,” or “insertion errors.” Some of these result from the stochastic skipping of bases in ratcheting or auto-ratcheting, damage to the arresting constructs, or other synthesis issues. As an example, mismatch errors may occur in the readout due to noise in the current traces. For example, the readout may interpret a reporter signal (voltage, current, etc.) as that of another reporter signal where the signals are not sufficiently separated or defined. Non-exhaustive examples of noise in nanopore sequencing are a temporarily clogged pore due to contaminants in solution, an ARC becoming temporarily stuck in the pore leading to a shift in current level, bilayer membrane instability, or a poor signal-to-noise ratio (SNR). For example, one error in nanopore sequencing relates to homopolymer sequences, where two nucleobases of the same type are adjacent to each other. In several different scenarios, as discussed further herein, the readout of the polynucleotide sequence may incorrectly interpret the same two nucleobases adjacent to each other as a single nucleobase, in what may be referred to as a “deletion error.” This is undesirable and can result in poor sequencing resolution.

Therefore, the disclosed examples provide means to reduce the errors in the readout process, for example by an order of magnitude or more. For example, double redundancy coding and arresting region modifications may be incorporated in various sequences of the cyclic loop in order to inhibit or prevent deletion errors, mismatch errors, and increase the signal to noise ratio.

In some aspects, the disclosed technology provides systems, devices, kits, and methods that employ cyclic loop “tick marks” that encode a nucleotide with a unique fingerprint that is unique from the reporting/encoding region of the nucleotide and provides redundancy in the readout of an individual nucleotide. In some examples a tick mark may be placed either before or after a coding region in a cyclic loop. In some aspects the systems, devices, kits, and methods also relate to optimized sequences for arresting constructs, encoding/reporter regions, and spacer regions for each nucleotide. In some instances, the sequences may be unique to each nucleotide, or may be pre-defined based upon the type of nanopore sequencing event.

The systems disclosed herein may be prepared to allow parallel reads in multiple nanopores, such as thousands or millions of nanopores. Accordingly, components of any system may be functionally duplicated to multiply sequencing throughput. Any system herein may also be adapted with microfluidics or automation.

The systems, devices, kits, and methods disclosed herein each have several aspects, no single one of which necessarily is solely responsible for their desirable attributes. Without limiting the scope of the claims, some prominent features will now be discussed briefly. Numerous other examples are also contemplated, including examples that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

Some examples herein provide a method of sequencing a polynucleotide. The method may include (i) disposing a polymer through a nanopore having a first side, a second side, and an aperture extending through the first and second sides, such that a first end of the polymer is on the first side of the nanopore, and a second end of the polymer is on the second side of the nanopore. The polymer encodes a sequence of a polynucleotide and includes a sequence of monomer units coupled to one another. Each of the monomer units encodes an identity of a nucleotide in the polynucleotide and includes a first reporter moiety; a second reporter moiety; a first arresting construct; and a second arresting construct. The method may include (ii) translocating the first reporter moiety of one of the monomer units into the aperture. The method may include (iii) measuring a first value of an electrical property of the first reporter moiety within the aperture while the first arresting construct pauses translocation of the first reporter moiety. The method may include (iv) translocating the second reporter moiety of the monomer unit into the aperture. The method may include (v) measuring a second value of an electrical property of the second reporter moiety within the aperture while the second arresting construct pauses translocation of the second reporter moiety. The method may include (vi) repeating operations (ii) through (v) for additional monomer units. The method may include (vii) using the first value and the second value for each of the monomer units to: identify the nucleotide encoded by that monomer unit; and distinguish the nucleotide encoded by that monomer unit from the nucleotides respectively encoded by adjacent monomer units, including by any adjacent monomer units that encode the same type of nucleotide as that monomer unit.

In some examples, translocating the first reporter moiety of that monomer unit into the aperture includes applying a first stimulus, and translocating the second reporter moiety of that monomer unit into the aperture includes applying a second stimulus. In some examples, the second stimulus is applied at a different time than the first stimulus. In some examples, the first stimulus and the second stimulus are of substantially the same magnitude as one another.

In some examples, the first reporter moiety is translocated out of the aperture using a constant stimulus, and the second reporter moiety is translocated out of the aperture using the constant stimulus.

In some examples, measuring the first value includes characterizing a first electrical current, ionic current, electrical resistance, or electrical voltage drop across the nanopore while the first reporter moiety is within the aperture; and measuring the second value includes characterizing a second electrical current, ionic current, electrical resistance, or electrical voltage drop across the nanopore while the second reporter moiety is within the aperture.

In some examples, the first reporter moiety uniquely identifies the nucleotide encoded by that monomer unit and the second reporter moiety does not uniquely identify the nucleotide encoded by that monomer unit; or the second reporter moiety uniquely identifies the nucleotide encoded by that monomer unit and the first reporter moiety does not uniquely identify the nucleotide encoded by that monomer unit.

In some examples, the first reporter moiety uniquely identifies the nucleotide encoded by that monomer unit; and the second reporter moiety uniquely identifies the nucleotide encoded by that monomer unit. In some examples, for an additional one of the monomer units, a single deletion error causes the first value to be measured and the second value not to be measured, or causes the first value not to be measured and the second value to be measured. The method further may include using the measured first value to uniquely identify the nucleotide and the non-measurement of the second value to identify that the single deletion error occurred; or using the measured second value to uniquely identify the nucleotide and the non-measurement of the first value to identify that the single deletion error occurred.

In some examples, for an additional one of the monomer units, a single insertion error causes the first value to be measured more than once, or causes the second value to be measured more than once. The method further may include using the twice measured first value to uniquely identify the nucleotide and to identify that the single insertion error occurred; or using the twice measured second value to uniquely identify the nucleotide to identify that the single insertion error occurred.

In some examples, the first and second reporter moieties, alone, do not uniquely identify the nucleotide encoded by that monomer unit; and the first and second reporter moieties, together, uniquely identify the nucleotide encoded by that monomer unit.

In some examples, each of the monomer units further includes a third reporter moiety and a third arresting construct. The first, second, and third reporter moieties, alone, do not uniquely identify the nucleotide encoded by that monomer unit; and the first, second, and third reporter moieties, together, uniquely identify the nucleotide encoded by that monomer unit.

In some examples, each of the monomer units further includes a third reporter moiety, a fourth reporter moiety, and a third arresting construct. The first, second, third, and fourth reporter moieties, alone, do not uniquely identify the nucleotide encoded by that monomer unit; and the first, second, third, and fourth reporter moieties, together, uniquely identify the nucleotide encoded by that monomer unit. In some examples, each of the monomer units further includes a fifth reporter moiety, a fourth arresting construct, and a fifth arresting construct. The fifth reporter moiety does not uniquely identify the nucleotide encoded by that monomer unit.

In some examples, the method further includes, before (vi) repeating operations (ii) through (v) for additional monomer units: (viii) translocating the second reporter moiety of that monomer unit out of the aperture while translocating that first reporter moiety into the aperture. The method further may include (ix) measuring a third value of an electrical property of that first reporter moiety within the aperture while the second arresting construct pauses translocation of that first reporter moiety. The method further may include (x) using the first, second, and third values together to: identify the nucleotide encoded by that monomer unit; and distinguish the nucleotide encoded by that monomer unit from the nucleotides respectively encoded by adjacent monomer units, including by any adjacent monomer units that encode the same type of nucleotide as that monomer unit.

In some examples, the method further includes, after (vi) repeating operations (ii) through (v) for additional ones of the monomer units: translocating a plurality of the monomer units to the first side of the nanopore, and then repeating operations (ii) through (vii) to obtain a plurality of additional values characterizing the polynucleotide encoded by the polymer.

In some examples, the first arresting construct is disposed between the first reporter moiety and a nucleobase of the monomer unit, or the first arresting construct is disposed between the second reporter moiety and a phosphate group of the monomer unit.

In some examples, the second arresting construct is disposed between the first reporter moiety and the second reporter moiety or between the first reporter moiety and the base.

In some examples, operations (ii) and (iii) are performed before operations (iv) and (v).

In some examples, operations (ii) and (iii) are performed after operations (iv) and (v).

Some examples herein provide a polymer encoding a sequence of a polynucleotide. The polymer includes a sequence of monomer units coupled to one another. Each of the monomer units encodes an identity of a nucleotide in the polynucleotide and includes: a first reporter moiety; a second reporter moiety; a first arresting construct; and a second arresting construct.

In some examples, the polymer includes four different types of the monomer units. A first type of the monomer units corresponds to a first type of nucleotide. A second type of the monomer units corresponds to a second type of nucleotide. A third type of the monomer units corresponds to a third type of nucleotide. A fourth type of the monomer units corresponds to a fourth type of nucleotide. In some examples, the first type of the monomer units corresponds to A, the second type of the monomer units corresponds to C, the third type of the monomer units corresponds to G, and the fourth type of the monomer units corresponds to T or U.

In some examples, the first reporter moieties of the first, second, third, and fourth types of the monomer units are of different types than one another. In some examples, at least some of the second reporter moieties of the first, second, third, and fourth types of the monomer unit are of the same type as one another. In some examples, the second reporter moieties of the first, second, third, and fourth types of the monomer unit are of the same type as one another.

In some examples, each of the monomer units further includes a third reporter moiety; and a third arresting construct. In some examples, each of the monomer units further includes a fourth reporter moiety. In some examples, within each one of the monomer units, at least two of the first, second, third, and fourth reporter moieties within that monomer unit are of different types than one another. In some examples, within each one of the monomer units, at least three of the first, second, third, and fourth reporter moieties within that monomer unit are of different types than one another. In some examples, within each one of the monomer units, all four of the first, second, third, and fourth reporter moieties within that monomer unit are of different types than one another. In some examples, the third arresting construct, the third reporter moiety, and the fourth reporter moiety are disposed between the first reporter moiety and the second reporter moiety.

In some examples, the first reporter moiety has a first electrical characteristic, and the second reporter moiety has a second electrical characteristic that is different from the first electrical characteristic.

In some examples, the first arresting construct is of the same type as the second arresting construct.

In some examples, the first arresting construct includes a peptide.

In some examples, the second arresting construct includes a peptide.

In some examples, each of the monomer units further includes a spacer disposed between the first reporter moiety and the second reporter moiety of the adjacent monomer unit.

In some examples, the first arresting construct is disposed between the first reporter moiety and a nucleobase of that monomer unit, or the first arresting construct is disposed between the second reporter moiety and a phosphate group of the monomer unit.

In some examples, the second arresting construct is disposed between the first reporter moiety and the second reporter moiety or between the first reporter moiety and the base.

Some examples herein provide a polymer encoding a sequence of a polynucleotide.

The polymer includes a sequence of monomer units coupled to one another. Each of the monomer units encodes an identity of a nucleotide in the polynucleotide and includes a first reporter moiety; a second reporter moiety having a different electrical characteristic than the first reporter moiety; and a first arresting construct.

In some examples, the first arresting construct is disposed between the first reporter moiety and the second reporter moiety of an adjacent monomer unit.

In some examples, each of the monomer units further includes a second arresting construct. In some examples, the second arresting construct is disposed between the first reporter moiety and the second reporter moiety.

In some examples, the polymer includes a first steric lock coupled to the first end of the polymer, and a second steric lock coupled to the second end of the polymer.

Some examples herein provide a composition. The composition may include a nanopore having a first side, a second side, and an aperture extending through the first and second sides. The composition further may include any of the polymers provided herein. A first end of the polymer may be on the first side of the nanopore, and a second end of the polymer may be on the second side of the nanopore.

Some examples herein provide a device that includes such a composition, and circuitry configured to implement any of the methods provided herein.

Some examples herein provide a nucleotide. The nucleotide may include a sugar. The nucleotide may include a nucleobase coupled to the sugar. The nucleotide may include an alpha phosphate group coupled to the sugar. The nucleotide may include a first reporter moiety coupled to the nucleobase. The nucleotide may include a second reporter moiety coupled the alpha phosphate group. The nucleotide may include a first arresting construct, The nucleotide may include a second arresting construct.

In some examples, the first arresting construct is disposed between the first reporter moiety and the second reporter moiety.

In some examples, the second arresting construct is either (i) disposed between the first reporter moiety and the nucleobase or (ii) disposed between the second reporter moiety and the alpha phosphate group.

Disclosed herein includes a cyclic loop nucleotide including a cyclic loop modification bridging a nucleobase and a phosphate group, wherein the cyclic loop modification includes a reporter encoding the identity of the nucleobase, an arresting construct adjacent to the reporter, a tick mark, and one or more spacer regions.

Disclosed herein also includes a cyclic loop nucleotide, wherein the cyclic loop modification further includes a second arresting construct adjacent to the tick mark.

In some aspects, the techniques described herein relate to a cyclic loop nucleotide, wherein the tick mark provides a signal that is different from that of any of the reporters encoding each nucleobase.

In some aspects, the techniques described herein relate to a cyclic loop nucleotide, wherein the signal provided by the tick mark is unique to the nucleobase.

In some aspects, the techniques described herein relate to a cyclic loop nucleotide, further including a first conjugating moiety connection a first end of the cyclic loop modification to the nucleobase, a second conjugating moiety connecting a second end of the cyclic loop modification to the phosphate group, and optionally a first linker between the first conjugating moiety and the nucleobase and a second linker between the second conjugating moiety and the phosphate group.

In some aspects, the techniques described herein relate to a cyclic loop nucleotide having one of the following structures:

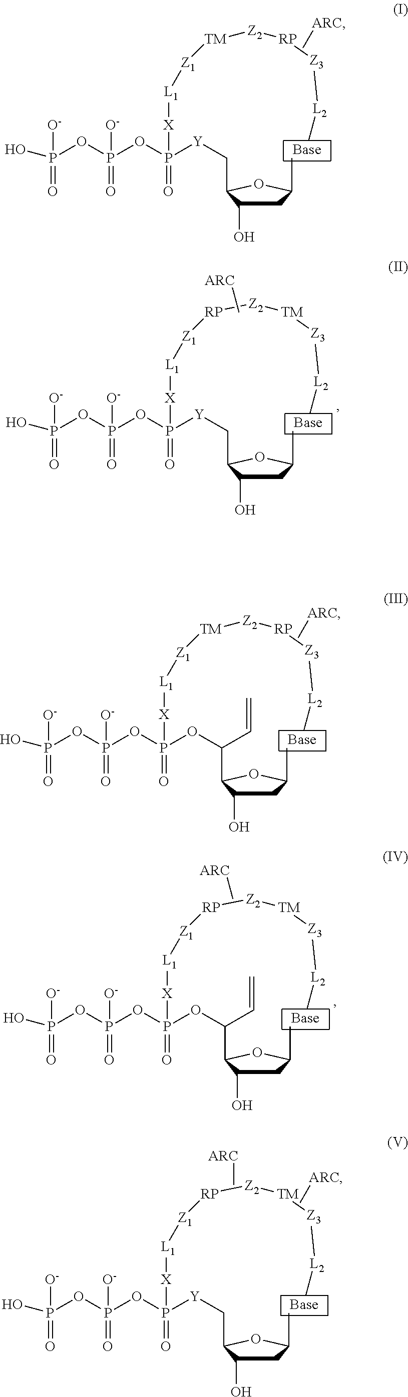

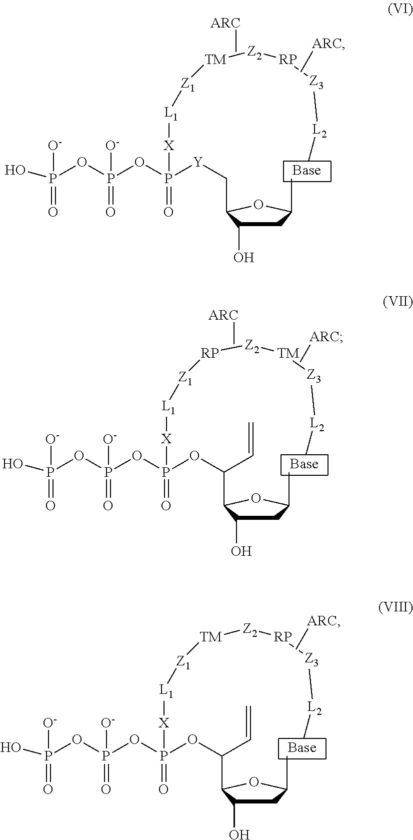

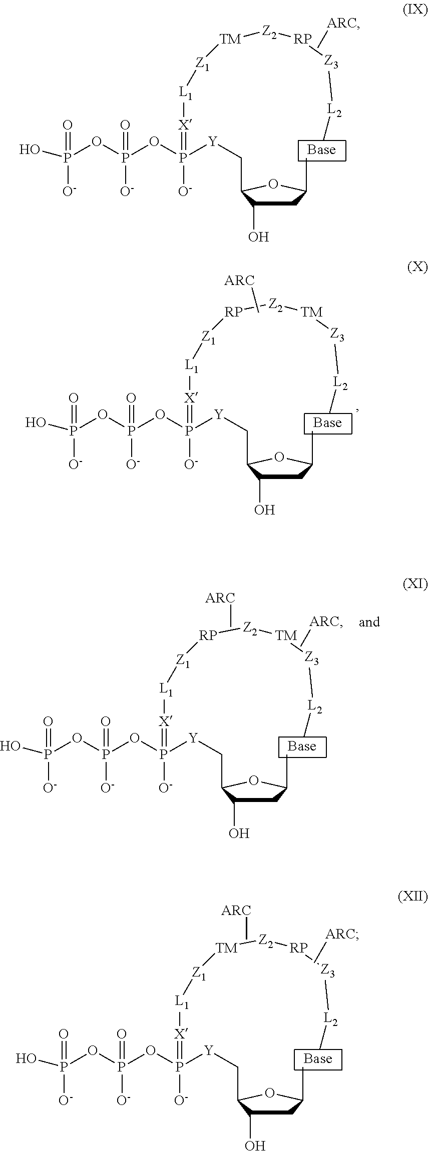

wherein X is —O—, —CH₂—, —NSO₂—, —NH—,

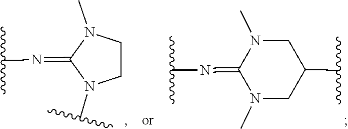

X′ is ═N—SO2-; ═NH—CO—, or

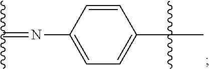 [SMILES: *c1ccc(N=*)cc1]

Y is —O—, —S—, —NH—, or —Se—; Base is the nucleobase; L₁ and L₂ are each a linking group; Z₁, Z₂, and Z₃ are each a spacer or absent; RP is the reporter encoding the nucleobase; TM is the tick mark; and ARC is the arresting construct.

In some aspects, the techniques described herein relate to a cyclic loop nucleotide, wherein each of L₁ and L₂ independently includes a conjugating moiety selected from the group consisting of amine-NHS ester, amine-imidoester, amine-pentafluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene.

In some aspects, the techniques described herein relate to a cyclic loop nucleotide, wherein each of L₁ and L₂ independently further includes an additional linker between the conjugating moiety and X.

In some aspects, the techniques described herein relate to a cyclic loop nucleotide, wherein the linker are independently selected from the group consisting of polynucleotide having 10 to 100 repeating units, polypeptide having 10 to 100 repeating units, alkyl chains having 5 to 50 carbons, hydrophilic polymers having 10 to 100 repeating units including polyethylene glycol, polyvinyl alcohol, polyacrylamide, polyvinylpyrrolidone, polystyrene sulfonate, or polyethyleneimine, hydrophobic polymers having 10 to 100 repeating units including polylactic acid, polymethylmethacrylate, or polystyrene, and combinations thereof.

In some aspects, the techniques described herein relate to a cyclic loop nucleotide, wherein the spacer includes a oligonucleotide or modified oligonucleotide or phosphoramidite analogs having 1 to 100 repeating units, polypeptide having 1 to 100 repeating units, alkyl chains having 5 to 50 carbons, hydrophilic polymers having 1 to 100 repeating units selected form the group consisting of polyethylene glycol, polyvinyl alcohol, polyacrylamide, polyvinylpyrrolidone, polystyrene sulfonate, and polyethyleneimine, hydrophobic polymers having 1 to 100 repeating units selected from the group consisting of polylactic acid, polymethylmethacrylate, and polystyrene, and combinations thereof.

In some aspects, the techniques described herein relate to a cyclic loop nucleotide, wherein the tick mark is selected from the group consisting of aliphatic chains, synthetic polymers, polyphosphate, polypeptide, oligonucleotides, and modified oligonucleotides.

In some aspects, the techniques described herein relate to a cyclic loop nucleotide, wherein the reporter includes oligonucleotides, modified oligonucleotides or phosphoramidite analogs having 1 to 100 repeating units, or polypeptides having 1 to 100 repeating units.

In some aspects, the techniques described herein relate to a cyclic loop nucleotide, wherein the arresting construct includes a synthetic hydrophobic polymer, a synthetic hydrophilic polymer, an oligonucleotide/polynucleotide, a peptide/polypeptide, or combinations thereof.

In some aspects, the techniques described herein relate to a cyclic loop nucleotide including a cyclic loop modification bridging a nucleobase and a phosphate group, wherein the cyclic loop modification includes two or more reporters in combination encoding the nucleobase, two or more arresting constructs adjacent to the two or more reporters, and one or more spacer regions.

In some aspects, the techniques described herein relate to a cyclic loop nucleotide, wherein the two or more reporters are independently selected from three or more unique reporter moieties.

In some aspects, the techniques described herein relate to a cyclic loop nucleotide, wherein the cyclic loop modification includes 2, 3, 4, or 6 reporters independently selected from 4 unique reporter moieties.

In some aspects, the techniques described herein relate to a cyclic loop nucleotide, wherein the cyclic loop modification includes 2 reporters independently selected from 8 unique reporter moieties.

In some aspects, the techniques described herein relate to a cyclic loop nucleotide, wherein the cyclic loop modification includes 4 or 5 reporters independently selected from 3 unique reporter moieties.

In some aspects, the techniques described herein relate to a cyclic loop nucleotide, wherein the cyclic loop modification includes 5 reporters independently selected from 5 unique reporter moieties.

In some aspects, the techniques described herein relate to a cyclic loop nucleotide, further including a first conjugating moiety connection a first end of the cyclic loop modification to the nucleobase, a second conjugating moiety connecting a second end of the cyclic loop modification to the phosphate group, and optionally a first linker between the first conjugating moiety and the nucleobase and a second linker between the second conjugating moiety and the phosphate group.

In some aspects, the techniques described herein relate to a cyclic loop nucleotide having one of the following structures:

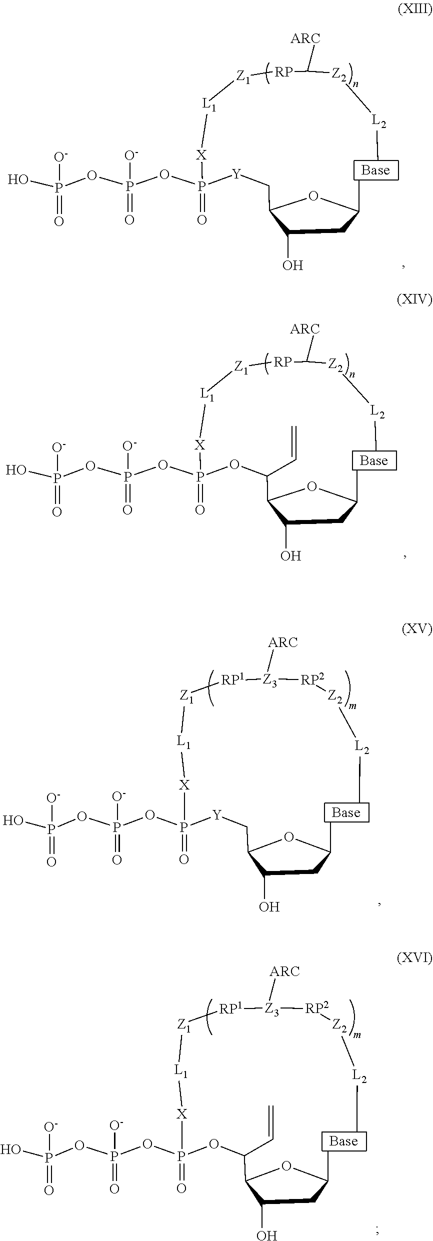

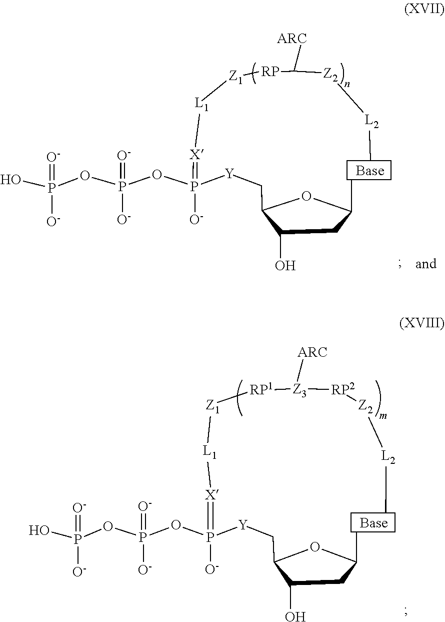

wherein X is —O—, —CH₂—, —NSO₂—, —NH—,

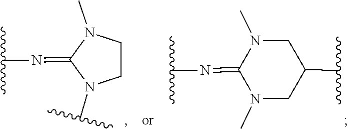

X′ is ═N—SO₂—; ═NH—CO—, or

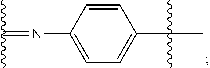 [SMILES: *c1ccc(N=*)cc1]

Y is —O—, —S—, —NH—, or —Se—; Base is the nucleobase; L₁ and L₂ are each a linking group; m or n is a positive integer; Z₁, Z₂, and Z₃ are each a spacer or absent; RP, RP¹ and RP² are each the reporter, wherein a combination of all the RP or a combination of all the RP¹ and RP² encodes the nucleobase; and ARC is an arresting construct.

In some aspects, the techniques described herein relate to a cyclic loop nucleotide, wherein each of L₁ and L₂ independently includes a conjugating moiety selected from the group consisting of amine-NHS ester, amine-imidoester, amine-pentafluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene.

In some aspects, the techniques described herein relate to a cyclic loop nucleotide, wherein each of L₁ and L₂ independently further includes a linker between the conjugating moiety and X or X′.

In some aspects, the techniques described herein relate to a cyclic loop nucleotide, wherein the linker are independently selected from the group consisting of polynucleotide having 1 to 100 (e.g., 10 to 100 repeating units), polypeptide having 1 to 100 (e.g., 10 to 100) repeating units, alkyl chains having 5 to 50 carbons, hydrophilic polymers having 1 to 100 (e.g., 10 to 100) repeating units including polyethylene glycol, polyvinyl alcohol, polyacrylamide, polyvinylpyrrolidone, polystyrene sulfonate, or polyethyleneimine, hydrophobic polymers having 1 to 100 (e.g., 10 to 100) repeating units including polylactic acid, polymethylmethacrylate, or polystyrene, and combinations thereof.

In some aspects, the techniques described herein relate to a cyclic loop nucleotide, wherein the spacer includes a oligonucleotide or modified oligonucleotide or phosphoramidite analogs having 1 to 100 repeating units, polypeptide having 1 to 100 repeating units, alkyl chains having 5 to 50 carbons, hydrophilic polymers having 1 to 100 repeating units selected form the group consisting of polyethylene glycol, polyvinyl alcohol, polyacrylamide, polyvinylpyrrolidone, polystyrene sulfonate, and polyethyleneimine, hydrophobic polymers having 1 to 100 repeating units selected from the group consisting of polylactic acid, polymethylmethacrylate, and polystyrene, and combinations thereof.

In some aspects, the techniques described herein relate to a cyclic loop nucleotide, wherein the tick mark is selected from the group consisting of aliphatic chains, synthetic polymers, polyphosphate, oligonucleotide/polynucleotide, modified oligonucleotide, a peptide/polypeptide, or combinations thereof.

In some aspects, the techniques described herein relate to a cyclic loop nucleotide, wherein the reporter includes oligonucleotides, modified oligonucleotides or phosphoramidite analogs having 1 to 100 repeating units, or polypeptides having 1 to 100 repeating units.

In some aspects, the techniques described herein relate to a cyclic loop nucleotide, wherein the arresting construct includes a synthetic hydrophobic polymer, a synthetic hydrophilic polymer, an oligonucleotide/polynucleotide, a peptide/polypeptide, or combinations thereof.

In some aspects, the techniques described herein relate to a method for determining a sequence of a target polynucleotide in a nanopore-based sequencing system, the method comprising: providing the target polynucleotide comprising a plurality of any one of the cyclic loop nucleotides disclosed herein; cleaving a cleavable bond on each of the plurality of cyclic loop nucleotide between the phosphate and a nucleoside, thereby elongating the target polynucleotide to form an elongated polymer, wherein the elongated polymer includes the cyclic loop modification associated with each nucleoside; applying a voltage to cause the elongated polymer to insert into and translocate through a nanopore; and reading a plurality of current signals as the elongated polymer translocate through the nanopore, wherein each of the current signal or a group of current signals correlates to the identity of the nucleobase.

In some aspects, the techniques described herein relate to a kit for performing a method for determining a sequence of a polynucleotide in a nanopore-based sequencing system, the kit comprising any one of the cyclic loop nucleotides disclosed herein.

In some aspects, the techniques described herein relate to a system for determining a sequence of a target polynucleotide, the system configured to perform a method according to any of the methods disclosed herein.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIGS. 1A and 1B schematically illustrate an example of sequencing an elongated polynucleotide and the elongation of a modified nucleotide with a cyclic loop.

FIG. 2 schematically illustrates a sample workflow for a nanopore sequencing event.

FIG. 3 illustrates an example modified nucleobase with a cyclic loop.

FIG. 4 illustrates a sample readout process for the example modified nucleobase in FIG. 3.

FIG. 5 illustrates an example modified nucleobase with a “tick mark.”

FIG. 6A illustrates a sample readout process for an example homopolymer polynucleotide comprising the example modified nucleobase in FIG. 3.

FIG. 6B illustrates a sample readout process for the example modified nucleobase in FIG. 5.

FIG. 7 demonstrates an example sequence associated with the modified nucleobase in FIG. 5 and potential errors associated therewith.

FIGS. 8A and 8B illustrate sample readout processes for an example F-2-8 EC reporter.

FIG. 9A illustrates an error correcting process associated with an example strand of F-2-8 EC reporters.

FIG. 9B illustrates an example sequence of example F-2-8 EC reporters and potential errors associated therewith.

FIGS. 10A and 10B illustrate a comparison between an example F-2-5 reporter (tick mark reporter) and an example F-2-8 EC reporter.

FIG. 11 illustrates a structural example of an F-3-4 EC reporter modified nucleobase.

FIG. 12 illustrates a structural example of an F-5-5 EC reporter modified nucleobase.

FIGS. 13A, 13B, and 13C illustrate a comparison of example F-2-8, F-3-4, and F-5-5 EC reporters.

FIG. 14 illustrates a structural example of a B-6-4 EC reporter.

FIG. 15 illustrates a plot of example candidate current reporter structures and their output signals.

FIG. 16 illustrates an example signal to noise ratio versus reporter current separations.

FIG. 17 illustrates a structural example of a transient tick mark nucleobase.

FIG. 18 illustrates example error reduction with a tick mark scheme.

FIG. 19 illustrates example transient tick mark candidates and data associated therewith.

FIGS. 20A-20E illustrate an example modified cyclic loop for a transient tick mark.

FIG. 21 illustrates an example flow of operations in a method for sequencing a polynucleotide using a polymer that encodes a sequence of the polynucleotide.

FIG. 22 illustrates an example F-4-3 EC reporter modified nucleobase.

FIG. 23A schematically illustrates an example modified nucleobase with an asymmetric cyclic loop including a single arresting construct and a single reporter moiety.

FIG. 23B schematically illustrates a polymer including a monomer unit with the cyclic loop of FIG. 23A, disposed through a nanopore.

FIG. 23C schematically illustrates a polymer including a monomer unit with an alternative, symmetric cyclic loop, disposed through a nanopore.

FIGS. 23D-1 through 23D-6 schematically illustrate an example implementation of a modified nucleobase with an symmetric cyclic loop.

FIG. 24A schematically illustrates a polymer including a monomer unit with an asymmetric cyclic loop including two arresting constructs and two reporter moieties.

FIG. 24B schematically illustrates a polymer including an alternative monomer unit with a symmetric cyclic loop including two arresting constructs.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or system, the term “comprising” means that the compound, composition, or system includes at least the recited features or components, but may also include additional features or components.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “nanopore” is intended to mean a hollow structure discrete from, or defined in, and extending across a membrane or substrate. A nanopore includes an aperture that permits molecules to cross therethrough from a first side of the nanopore to a second side of the nanopore, in which a portion of the aperture of a nanopore has a width of 100 nm or less, e.g., 10 nm or less, or 2 nm or less. The aperture extends through the first and second sides of the nanopore. The nanopore permits ions, electric current, and/or fluids to cross from one side of the membrane to the other side of the membrane. For example, a membrane that inhibits the passage of ions or water-soluble molecules can include a nanopore structure that extends across the membrane to permit the passage, through a nanoscale opening (aperture) extending through the nanopore structure, of the ions or water-soluble molecules (such as amino acids or nucleotides) from one side of the membrane to the other side of the membrane. The diameter of the nanoscale opening extending through the nanopore structure can vary along its length (i.e., from one side of the membrane to the other side of the membrane), but at any point is on the nanoscale (i.e., from about 1 nm to about 1000 nm, or from about 1 nm to about 100 nm, or from about 1 nm to about 10 nm, or any other dimension less than 1000 nm). In some examples, a nanopore refers to a pore having an opening with a diameter at its most narrow point of about 0.3 nm to about 2 nm. Optionally, a portion of the aperture can be narrower than one or both of the first and second sides of the nanopore, in which case that portion of the aperture can be referred to as a “constriction” or a “readhead.” Alternatively or additionally, the aperture of a nanopore, or the constriction of a nanopore (if present), or both, can be greater than 0.1 nm, 0.5 nm, 1 nm, 10 nm or more. A nanopore can include multiple constrictions, e.g., at least two, or three, or four, or five, or more than five constrictions.

Examples of the nanopore include, for example, biological nanopores, solid-state nanopores, and biological and solid-state hybrid nanopores. In some examples, a nanopore may be a solid-state nanopore, a graphene nanopore, or an elastomer nanopore, or may be a naturally-occurring or recombinant protein that forms a tunnel upon insertion into a bilayer, thin film, membrane, or solid-state aperture, also referred to as a protein pore or protein nanopore herein (e.g., a transmembrane pore). If the protein inserts into the membrane, then the protein may be referred to as a tunnel-forming protein.

As used herein, the term “diameter” is intended to mean a longest straight line inscribable in a cross-section of a nanoscale opening through a centroid of the cross-section of the nanoscale opening. It is to be understood that the nanoscale opening may or may not have a circular or substantially circular cross-section (where in some examples, the cross-section of the nanoscale opening is substantially parallel with the cis/trans electrodes and/or is substantially perpendicular to an axis of the opening). Further, the cross-section may be regularly shaped or irregularly shaped.

As used herein, the term “biological nanopore” is intended to mean a nanopore whose structure portion is made from materials of biological origin. Biological origin refers to a material derived from or isolated from a biological environment such as an organism or cell, or a synthetically manufactured version of a biologically available structure. Biological nanopores include, for example, polypeptide nanopores and polynucleotide nanopores.

As used herein, the term “polypeptide nanopore” is intended to mean a protein/polypeptide that extends across the membrane, and permits ions, electric current, polymers such as DNA or peptides, or other molecules of appropriate dimension and charge, and/or fluids to flow therethrough from one side of the membrane to the other side of the membrane. A polypeptide nanopore can be a monomer, a homopolymer, or a heteropolymer. Structures of polypeptide nanopores include, for example, an α-helix bundle nanopore and a β-barrel nanopore. Example polypeptide nanopores include α-hemolysin, Mycobacterium smegmatis porin A (MspA, which also may be referred to as Msp porin), gramicidin A, maltoporin, OmpF, OmpC, PhoE, Tsx, F-pilus, CsgG, etc. The protein α-hemolysin is found naturally in cell membranes, where the α-hemolysin acts as a pore for ions or molecules to be transported in and out of cells. Mycobacterium smegmatis porin A (MspA) is a membrane porin produced by Mycobacteria, which allows hydrophilic molecules to enter the bacterium. MspA forms a tightly interconnected octamer and transmembrane beta-barrel that resembles a goblet and contains a central pore. For further details regarding α-hemolysin, see U.S. Pat. No. 6,015,714, the entire contents of which are incorporated by reference herein. For further details regarding SP1, see Wang et al., Chem. Commun., 49:1741-1743 (2013), the entire contents of which are incorporated by reference herein. For further details regarding MspA, see Butler et al., “Single-molecule DNA detection with an engineered MspA protein nanopore,” Proc. Natl. Acad. Sci. 105:20647-20652 (2008) and Derrington et al., “Nanopore DNA sequencing with MspA,” Proc. Natl. Acad. Sci. USA, 107:16060-16065 (2010), the entire contents of both of which are incorporated by reference herein. Other nanopores include, for example, the MspA homolog from Norcadia farcinica, and lysenin. For further details regarding lysenin, see PCT Publication No. WO 2013/153359, the entire contents of which are incorporated by reference herein. For further details regarding aerolysin, see Cao et al., “Single-molecule sensing of peptides and nucleic acids by engineered aerolysin nanopores,” Nature Communications 10: Article number: 4918 (2019), the entire contents of which are incorporated by reference herein.

A polypeptide nanopore can be synthetic. A synthetic polypeptide nanopore includes a protein-like amino acid sequence that does not occur in nature. The protein-like amino acid sequence may include some of the amino acids that are known to exist but do not form the basis of proteins (i.e., non-proteinogenic amino acids). The protein-like amino acid sequence may be artificially synthesized rather than expressed in an organism and then purified/isolated.

As used herein, a “peptide” refers to two or more amino acids joined together by an amide bond (that is, a “peptide bond”). Peptides comprise up to or include 50 amino acids. Peptides may be linear or cyclic. Peptides may be α, β, γ, δ, or higher, or mixed. Peptides may comprise any mixture of amino acids as defined herein, such as comprising any combination of D, L, α, β, γ, δ, or higher amino acids.

As used herein, a “protein” refers to an amino acid sequence having 51 or more amino acids.

A “polynucleotide nanopore” is intended to mean a nanopore that is made from one or more nucleic acid polymers. A polynucleotide nanopore can include, for example, a polynucleotide origami.

As used herein, the term “solid-state nanopore” is intended to mean a nanopore whose structure portion is defined by a solid-state membrane and includes materials of non-biological origin (i.e., not of biological origin). A solid-state nanopore can be formed of an inorganic or organic material. Solid-state nanopores include, for example, silicon nitride (SiN), silicon dioxide (SiO₂), silicon carbide (SiC), hafnium oxide (HfO₂), molybdenum disulfide (MoS₂), hexagonal boron nitride (h-BN), or graphene. A solid-state nanopore may comprise an aperture formed within a solid-state membrane, e.g., a membrane including any such material(s).

In some examples, the nanopores disclosed herein may be hybrid nanopores. A “hybrid nanopore” refers to a nanopore including materials of both biological and non-biological origins. An example of a hybrid nanopore includes a polypeptide-solid-state hybrid nanopore and a polynucleotide-solid-state nanopore.

As used herein, “of biological origin” refers to material derived from or isolated from a biological environment such as an organism or cell, or a synthetically manufactured version of a biologically available structure.

As used herein, “solid-state” refers to material that is not of biological origin.

As used herein, the terms “membrane” and “barrier” refer to a non-permeable or semi-permeable barrier or other sheet that separates two liquid/gel chambers (e.g., a cis well and a fluidic cavity) which can contain the same compositions or different compositions therein. The permeability of the membrane to any given species depends upon the nature of the membrane. In some examples, the membrane may be non-permeable to ions, to electric current, and/or to fluids. For example, a membrane may be impermeable to ions (i.e., does not allow any ion transport therethrough), but may be at least partially permeable to water (e.g., water diffusivity ranges from about 40 μm/s to about 100 μm/s). For another example, a synthetic/solid-state membrane, one example of which is silicon nitride, may be impermeable to ions, electric charge, and fluids (i.e., the diffusion of all of these species is zero). Any suitable membrane may be used in accordance with the present disclosure, as long as the membrane can include a nanopore (e.g., transmembrane nanoscale opening) and can maintain a potential difference across the membrane. The membrane may be a monolayer or a multilayer membrane. A multilayer membrane includes two or more layers, each of which is a non-permeable or semi-permeable material. A membrane may be formed of materials of non-biological or biological origin.

An example membrane that is made from non-biological materials are block copolymer. The term is a “block copolymer” is intended to refer to a polymer having at least a first portion or block that includes a first type of monomer, and at least a second portion or block that is coupled directly or indirectly to the first portion and includes a second, different type of monomer. Block copolymers include, but are not limited to, diblock copolymers and triblock copolymers. A “diblock copolymer” is intended to refer to a block copolymer that includes a first and second blocks coupled directly or indirectly to one another. The first block may be hydrophilic and the second block may be hydrophobic, in which case the diblock copolymer may be referred to as an “AB” copolymer where “A” refers to the hydrophilic block and “B” refers to the hydrophobic block. A “triblock copolymer” is intended to refer to a block copolymer that includes a first, second, and third blocks coupled directly or indirectly to one another. The first and third blocks may include, or may consist essentially of, the same type of monomer (repeating unit) as one another, and the second block may include a different type of monomer (repeating unit). In one example, the first block may be hydrophilic, the second block may be hydrophobic, and the third block may be hydrophilic and includes the same type of monomer as the first block, in which case the triblock copolymer may be referred to as an “ABA” copolymer where “A” refers to the hydrophilic blocks and “B” refers to the hydrophobic block. The block copolymers may be formed into a bilayer membrane in which the hydrophilic blocks are position on the outward of the bilayer membrane and in which the hydrophobic blocks are positioned inward of the bilayer membrane.

Example hydrophilic A blocks include, but are not limited to, a polymer selected from the group consisting of: N-vinyl pyrrolidone, polyacrylamide, zwitterionic polymer (Zwitt), hydrophilic polypeptide, poly(ethylene glycol) (PEG), carbon-oxygen-nitrogen containing polymers (CₓO_{y}N_{z}), and combinations thereof. Example hydrophobic B blocks include, but are not limited to, poly(dimethylsiloxane) (PDMS), poly(isobutylene) (PiB), polybutadiene (PBd), polyisoprene, polymyrcene, polychloroprene, hydrogenated polydiene, fluorinated polyethylene, polypeptide, and combination thereof. Example block copolymers used to form a bilayer membrane include, but are not limited to, PDMS-ab-Zwitt, PiB-ab-Zwitt, PiB-ab-PEG, PiB-ab-(CₓO_{y}N_{z}), PDMS-ab-PEG, PDMS-ab-(CₓO_{y}N_{z}), PiB-aba-Zwitt, PiB-aba-PEG, PiB-aba-(CₓO_{y}N_{z}), and PDMS-aba-PEG, PDMS-ab-(CₓO_{y}N_{z}), and other suitable block copolymers.

An example membrane that is made from the material of biological origin includes a monolayer formed by a bolalipid. Another example membrane that is made from the material of biological origin includes a lipid bilayer. Suitable lipid bilayers include, for example, a membrane of a cell, a membrane of an organelle, a liposome, a planar lipid bilayer, and a supported lipid bilayer. A lipid bilayer can be formed, for example, from two opposing layers of phospholipids, which are arranged such that their hydrophobic tail groups face towards each other to form a hydrophobic interior, whereas the hydrophilic head groups of the lipids face outwards towards the aqueous environment on each side of the bilayer. Lipid bilayers also can be formed, for example, by a method in which a lipid monolayer is carried on an aqueous solution/air interface past either side of an aperture that is substantially perpendicular to that interface. The lipid is normally added to the surface of an aqueous electrolyte solution by first dissolving the lipid in an organic solvent and then allowing a drop of the solvent to evaporate on the surface of the aqueous solution on either side of the aperture. Once the organic solvent has at least partially evaporated, the solution/air interfaces on either side of the aperture are physically moved up and down past the aperture until a bilayer is formed. Other suitable methods of bilayer formation include tip-dipping, painting bilayers, and patch-clamping of liposome bilayers. Any other methods for obtaining or generating lipid bilayers may also be used.

In some examples, a solid-state membrane can be a monolayer, such as a coating or film on a supporting substrate (i.e., a solid support), or a freestanding element. The solid-state membrane can also be a composite of multilayered materials in a sandwich configuration. Any material not of biological origin may be used, as long as the resulting membrane can include a transmembrane nanoscale opening and can maintain a potential difference across the membrane. The membranes may include organic materials, inorganic materials, or both. Examples of suitable solid-state materials include, for example, microelectronic materials, insulating materials (e.g., silicon nitride (Si₃N₄), aluminum oxide (Al₂O₃), hafnium oxide (HfO₂), tantalum pentoxide (Ta₂O₅), silicon oxide (SiO₂), etc.), some organic and inorganic polymers (e.g., polyamide, plastics, such as polytetrafluoroethylene (PTFE), or elastomers, such as two-component addition-cure silicone rubber), and glasses. In some examples, the solid-state membrane can be made from a monolayer of graphene, which is an atomically thin sheet of carbon atoms densely packed into a two-dimensional honeycomb lattice, a multilayer of graphene, or one or more layers of graphene mixed with one or more layers of other solid-state materials. A graphene-containing solid-state membrane can include at least one graphene layer that is a graphene nanoribbon or graphene nanogap, which can be used as an electrical sensor to characterize the target polynucleotide. It is to be understood that the solid-state membrane can be made by any suitable method, for example, chemical vapor deposition (CVD). In an example, a graphene membrane can be prepared through either CVD or exfoliation from graphite.

As used herein, the term “nanopore sequencer” refers to any suitable device, such as any of the devices disclosed herein, that can be used for nanopore sequencing. In the examples disclosed herein, during nanopore sequencing, a membrane having a nanopore disposed or defined therethrough is disposed between first and second fluids (e.g., immersed in examples of the electrolyte disclosed herein) and a potential difference is applied across the membrane. In an example, the potential difference is an electric potential difference or an electrochemical potential difference. An electrical potential difference can be imposed across the membrane via a voltage source that injects or administers current to at least one of the ions of the electrolyte (first fluid) contained in the cis well or the electrolyte (second fluid) contained in one or more of the trans wells. An electrochemical potential difference can be established by a difference in ionic composition of the cis and trans wells in combination with an electrical potential. The different ionic composition can be, for example, different ions in each well or different concentrations of the same ions in each well. Apparatuses and methods include sequencing polynucleotides and sequencing polypeptides and include providing genomics analysis and proteomics analysis.

As used herein, “cis” refers to a first side of a nanopore opening. In some examples, “cis” refers to the side of the nanopore opening through which a polymer (e.g., a polynucleotide) enters the opening.

As used herein, “trans” refers to a second side of a nanopore opening which is opposite to the “cis” side. In some examples, “trans” refers to the side of a nanopore opening through which a polymer (e.g., a polynucleotide) exits the opening. In other examples, “trans” refers to the side of the nanopore opening through which a polymer (e.g., a polynucleotide) enters the opening. In some examples, a polymer (e.g., a polynucleotide) or a portion thereof (e.g., a monomer unit or a portion thereof) is repeatedly moved between the cis side and the trans side of the nanopore in a “flossing” type of motion.

As used herein, a “nucleotide” or “nucleic acid” includes a nitrogen containing a sugar, one or more phosphate groups, and in some examples also includes a nucleobase (e.g., heterocyclic base). Nucleotides are monomeric units of a nucleic acid sequence. Examples of nucleotides include, for example, deoxyribonucleotides, modified deoxyribonucleotides, ribonucleotides, modified ribonucleotides, peptide nucleotides, modified peptide nucleotides, modified phosphate sugar backbone nucleotides, and mixtures thereof. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides are natural nucleotides, but it is to be further understood that non-natural nucleotides, modified nucleotides or analogs of the aforementioned nucleotides can also be used.

Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleotides” or “modified nucleic acids.” Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”). Nucleotides thus may include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

As used herein, a “nucleoside” includes a nitrogen-containing heterocyclic base and a sugar. The base and the sugar are covalently bonded together through a β N-glycosidic linkage. A nucleoside differs from a nucleotide in that the sugar of the nucleoside is not directly linked to a phosphate group.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, FL), all of which are herein incorporated by reference in their entireties.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

Accordingly, in some examples, the term “polynucleotide” refers to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) or phosphorothiolate DNA. Unless otherwise indicated, a particular nucleic acid sequence includes the complementary sequence thereof. In some examples, the term “polynucleotide” is used herein to refer to a polymer that encodes a sequence of a polynucleotide. Such a polymer may include a plurality of monomer units, each of which encodes an identity of a nucleotide in a polynucleotide. Each of the monomer units of the polymer may include one or more reporter moieties and one or more arresting constructs (e.g., two or more reporter moieties, and two or more arresting constructs).

As used herein, the terms “encode” and “parse” are verbs referring to transferring from one format to another, and refer to transferring the genetic information of a polynucleotide (e.g., target template base sequence) into a polymer including an arrangement of reporter regions.

As used herein, the term “modified oligonucleotide” refers to a polymeric chain of nucleobases or nucleotides assembled with moieties comprising a modified nucleobase, one or more modified sugar rings (e.g., LNA, constraint ethyl, ethylene bridged, TNA, 2′-OMe, 2′-F, 2′-MOE) and/or one or more nucleobases attached to a scaffold (e.g. unlock, 4′-thio, CeNA (cyclohexene nucleic acid), HNA (hexitol nucleic acid), TNA (threose nucleic acid), GNA (glycol nucleic acid), or FNA (fluoronucleic acid)). In CeNA, the sugar backbone is replaced by a cyclohexene ring, which can influence the binding properties and stability of nucleic acid strands. HNA has a six-carbon sugar (hexitol) in its backbone instead of the five-carbon sugar (deoxyribose) found in DNA; this modification can enhance stability and binding affinity. TNA uses a threose sugar in its backbone, which is a four-carbon sugar; this makes TNA one of the simplest nucleic acid analogs, potentially useful in prebiotic chemistry and origin-of-life studies. GNA has a simpler, two-carbon glycol backbone; it is studied for its potential role in synthetic biology and as a primitive form of genetic material. FNA refers to nucleic acids with fluorinated sugar backbones; fluorination can enhance the stability of nucleic acids, making FNA useful in therapeutic and diagnostic applications.

As used herein, the term “phosphoramidite analogs” refers to any polymer synthesized using phosphoramidite or related chemistries resulting in the formation of phosphodiester, methylphosphonate, or phosphorothioate bonds between each moiety.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP. Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and Isopol™ SD+ polymerase. In specific, nonlimiting examples, the polymerase is selected from a group consisting of Bst, Bsu, and Phi29. Some polymerases have an activity that degrades the strand behind them’ (3′ exonuclease activity). Some useful polymerases have been modified, either by mutation or otherwise, to reduce or eliminat’ 3′ and/o′ 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NS5B protein. Example RNA Reverse Transcriptases. A non-limiting example list includes reverse transcriptases derived from Avian Myelomatosis Virus (AMV), Murine Moloney Leukemia Virus (MMLV) and/or the Human Immunodeficiency Virus (HIV), telomerase reverse transcriptases such as (hTERT), SuperScript™ III, SuperScript™ IV Reverse Transcriptase, ProtoScript® II Reverse Transcriptase.

In some examples, polymerases include, but are not limited to, DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase I, Klenow fragment, Thermophilus aquaticus DNA polymerase, Tth DNA polymerase, VentR® DNA polymerase (New England Biolabs), Deep VentR® DNA polymerase (New England Biolabs), Bst DNA Polymerase Large Fragment, Stoeffel Fragment, 90N DNA Polymerase, 90N DNA polymerase, Pfu DNA Polymerase, TfI DNA Polymerase, Tth DNA Polymerase, RepliPHI Phi29 Polymerase, Tii DNA polymerase, eukaryotic DNA polymerase beta, telomerase, Therminator™ polymerase (New England Biolabs), KOD HiFi™ DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting, and polymerases cited in US 2007/0048748, U.S. Pat. Nos. 6,329,178, 6,602,695, and 6,395,524 (incorporated by reference). These polymerases include wild-type, mutant isoforms, and genetically engineered variants.

In the context of a polypeptide, the terms “variant” and “derivative” as used herein refer to a polypeptide that includes an amino acid sequence of a polypeptide or a fragment of a polypeptide, which has been altered by the introduction of amino acid residue substitutions, deletions, or additions. A variant or a derivative of a polypeptide can be a fusion protein which contains part of the amino acid sequence of a polypeptide. The term “variant” or “derivative” as used herein also refers to a polypeptide or a fragment of a polypeptide, which has been chemically modified, e.g., by the covalent attachment of any type of molecule to the polypeptide. For example, but not by way of limitation, a polypeptide or a fragment of a polypeptide can be chemically modified, e.g., by glycosylation, acetylation, pegylation, phosphorylation, methylation, nitrosylation, amidation, derivatization by known protecting/blocking groups, proteolytic cleavage, linkage to a cellular ligand or other protein, etc. The variants or derivatives are modified in a manner that is different from naturally occurring or starting peptide or polypeptides, either in the type or location of the molecules attached. Variants or derivatives further include deletion of one or more chemical groups which are naturally present on the peptide or polypeptide. A variant or a derivative of a polypeptide or a fragment of a polypeptide can be chemically modified by chemical modifications using techniques known to those of skill in the art, including, but not limited to specific chemical cleavage, acetylation, formulation, metabolic synthesis of tunicamycin, etc. Further, a variant or a derivative of a polypeptide or a fragment of a polypeptide can contain one or more non-classical amino acids. A polypeptide variant or derivative may possess a similar or identical function as a polypeptide, or a fragment of a polypeptide described herein. A polypeptide variant or derivative may possess an additional or different function compared with a polypeptide or a fragment of a polypeptide described herein.

As used herein, a “moiety” is intended to refer to a portion of a molecule which has a functionality that is distinct from the functionality of another portion of a molecule. In some examples, a “moiety” is one of two or more parts into which something may be divided, such as, for example, the various parts of a molecule.

As used herein, a “reporter,” “reporter moiety,” “reporter region,” or “reporter element” refers to a structure which may be used to identify a nucleotide. A reporter may be composed of one or more reporter elements or reporter moieties. Reporters include what are known as “tags” and “labels,” and in some cases the term “reporter” may be used synonymously with the term “tag” or “label.” Reporters may serve to encode (parse) the identities of nucleotides in a polynucleotide. In some examples, reporters may include constituent sub-reporters. In some examples, multiple reporters may be used to identify, or may be coupled to or present on, a single nucleotide or oligonucleotide When present in the readhead of a nanopore, reporters provide distinctive and sometimes unique blockage currents at given read voltages. A combination of “reporter moieties”, or “reporter elements” may be referred to herein as a “barcode,” “barcode region,” “barcoding/reporter region,” “reporter/barcode,” “reporter barcode,” or the like. A “barcode” may be used to identify a nucleotide, a group of nucleotides (e.g. purines, or pyrimidines, or another suitable subset of nucleotides), or may be associated with a tick mark. In some instances a barcode may be identified by the plurality of signals it produces under applied bias when the barcode (or “reporter moieties”) are located in, or near, the nanopore constriction. A reporter that does not uniquely encode the identity of a nucleotide (that is, from which the identity of a nucleotide may not be uniquely determined, for example because some or all monomer units contain a reporter of that type) may be referred to herein in some examples as a “tick mark” or in other examples as an “error correcting reporter.”

As used herein, a “linker,” “linking group,” or “linker group” is a molecule or moiety that joins two molecules or moieties and provides spacing between the two molecules or moieties such that they are able to function in their intended manner. For example, a linker can comprise a diamine hydrocarbon chain that is covalently bound through a reactive group on one end to an oligonucleotide analog molecule and through a reactive group on another end to a solid support, such as, for example, a bead surface. Coupling of linkers to nucleotides and substrate constructs of interest can be accomplished through the use of coupling reagents that are known in the art (see, e.g., Efimov et al., Nucleic Acids Res. 27:4416-4426, 1999, the entire contents of which are incorporated by reference herein). Methods of derivatizing and coupling organic molecules are well known in the arts of organic and bioorganic chemistry. A linker may also be cleavable or reversible.

As used in herein, the term “tick mark” refers to a reporter (e.g., coding region) that produces a signal distinct from the signals produced by any of the reporters used to identify a given nucleotide, but from which the nucleotide may not be uniquely identified. For example, the bases A, T (or U), C, and G may each be configured to have at least one unique reporter associated with that base, as well as at least one tick mark which would have a signal distinct from the reporter signal signatures associated with either A, T (or U), C, or G. In some examples, a generic tick mark or a universal tick mark is a structure which produces the same single, distinct signal for each nucleobase, for example, when incorporated into the cyclic loops of all four nucleobases.

As used herein, the term “Error Correcting reporter” (EC reporter) refers to a coding region modification associated with a particular nucleobase that produces an order of signals to identify the passage of a nucleobase through a nanopore. An EC reporter may further be used to help identify the nucleobase or may be used to identify errors that can occur during the measurement of the signals associated with a reporter (e.g. skipping/deletion errors, insertion errors, mismatch errors, etc.). An EC reporter may include a type of tick mark. Various EC reporters may include additional signals (tick marks or otherwise) or a group of signals associated with each individual nucleobase, A, T (or U), C, or G. These groups of signals are constructed to imbue some kind of “error correcting”, “error minimizing”, or other similar desirable property for identifying the nucleotide. An example of such an error correcting property may include tolerance to deletion/skipping errors as provided by a universal tick mark, or may be provided by use of redundancy (e.g. repeated use) of reporter signals unique to a nucleobase.

As used herein, the term “Error Correcting nucleotide” (EC nucleotide) refers to a type of modified nucleotide (e.g. a cyclic loop nucleotide), and may contain a collection of EC reporters, arresting constructs, spacers, linkers, nucleobase, cyclic loop modifications, and cleavage groups to elongate the cyclic loop modification. The order of reporters on the EC nucleotide may be referred to as an EC code, which refers to the encoding scheme of the EC nucleotide that allows the nucleotide to be uniquely identified, as well as any associated errors during measurement of the signals associated with the nucleotide. EC nucleotides.

As used herein, the term “Error Correcting oligomer” (EC oligo) refers to a type of modified oligomer (e.g. such as a primer, or other short polynucleotide), that may contain a collection of EC reporters, arresting constructs, spacers, linkers, nucleobase, cyclic loop modifications, and cleavage groups to elongate the cyclic loop modification. The order of reporters on the EC oligomer may be referred to as an EC code, which refers to the encoding scheme of the EC nucleotide that allows the oligomer to be uniquely identified, as well as any associated errors during measurement of the signals associated with the nucleotide.

As used herein, the term “Transient Tick Mark” refers to a tick mark that may translocate through the readhead without having an associated arresting construct to slow down translocation of the tick mark. Typically a transient tick mark is not directly adjacent to an arresting construct in a cyclic loop. Transient tick marks may each produce unique signals associated with each nucleobase with a distinct signal from the reporter for each nucleobase.

As used herein, the term “arresting construct” or “ARC” refers to a structure that provides a resistance (in the form of a “holding force”) that slows and/or stops a polymer (e.g., polynucleotide) to translocate through the nanopore unless the resistance due to the arresting construct is overcome by a “driving force.” The resistance provided by the arresting construct is due to a property of the arresting construct (e.g., size, geometry, and/or non-covalent interaction with the nanopore). An arresting construct can operate as a ratchet or a brake for the polymer (e.g., polynucleotide) translocation through a nanopore.

As used herein, the term “arrest” generally means to stop and/or slow down. For example, when the translocation of a polymer (e.g., polynucleotide) through a nanopore is arrested, the relative motion of the polymer with respect to the nanopore may come to rest or may continue but with a slower speed. An arresting construct that arrests nucleotide translocation through a nanopore may serve to stop or slow down translocation compared to an unmodified nucleotide.

The application of the electric potential difference across a nanopore may facilitate the translocation of a polymer (e.g., polynucleotide) through the nanopore. One or more signals are generated that correspond to the translocation of the polymer (e.g., polynucleotide) through the nanopore. Illustratively, as a polymer (e.g., polynucleotide) translocates through the nanopore, the voltage or current across the membrane changes due to time-varying blockage of the constriction, for example. The signal from that change in voltage or current can be measured using any of a variety of methods. Each signal is unique to the species of nucleotide(s) (e.g., is unique to the reporter moiet(ies) encoding the identity of such nucleotide, for example cyclic loop modification(s) with reporter moiety region(s)) in the nanopore, such that the resultant signal can be used to determine a characteristic of the polynucleotide. For example, the identity of one or more species of nucleotide(s) that produces a characteristic signal can be determined.

As used herein, the term “signal” is intended to mean an indicator that represents information. Signals include, for example, an electrical signal and an optical signal. The term “electrical signal” refers to an indicator of an electrical quality that represents information. The indicator can be, for example, current, voltage, tunneling, resistance, potential, voltage, conductance, or a transverse electrical effect. An “electronic current” or “electric current” refers to a flow of electric charge. In an example, an electrical signal may be an electric current passing through a nanopore, and the electric current may flow when an electric potential difference is applied across the nanopore.

As used herein, the term “driving force” is intended to mean an electrical current that causes or allows a polynucleotide to translocate through the nanopore. In some examples, the electrical current may flow when an electric potential difference is applied across the nanopore.

As used herein, the term “holding force” is intended to mean a resistance that slows and/or stops a polynucleotide to translocate through the nanopore. In some examples, the holding force is overcome by the application of a driving force. Thus, the driving force overcomes/overrides the resistance that slows and/or stops a polynucleotide, thereby allowing the polynucleotide to translocate through the nanopore.

As used herein, “translocation,” means that a polymer (e.g., a polynucleotide) enters one side of an opening of a nanopore and move to and out of the other side of the opening. It is contemplated that any example herein comprising translocation may refer to electrophoretic translocation or non-electrophoretic translocation, unless specifically noted. In some examples, an electric field may be used to translocate a polymer through a nanopore. By “interacts,” it is meant that polymer moves into and, optionally, through the opening of a nanopore, where “through the opening” (or “translocates”) means to enter one side of the opening and move to and out of the other side of the opening. Optionally, methods that do not employ electrophoretic translocation are contemplated. In some examples, physical pressure causes a polymer to interact with, enter, or translocate (e.g., after alteration) through the opening. In some examples, a magnetic bead is attached to a polymer on the trans side, and magnetic force causes the polymer to interact with, enter, or translocate (after alteration) through the opening. Other methods for translocation include but not limited to gravity, osmotic forces, temperature, and other physical forces such as centripetal force.

In some examples, the polymer may enter one side of the nanopore and exit out the other side of the nanopore. In some examples, the polymer may move forward and backward within or through the nanopore. In some examples, the polymer may move through and out of the other side of the nanopore, but may not exit the nanopore completely.

A “vestibule” refers to the interior portion of a nanopore. In examples in which the nanopore is or includes an Msp porin (MspA), the “vestibule” may refer to the cone-shaped portion of the interior of the Msp porin, whose diameter generally decreases from one end to the other along a central axis, where the narrowest portion of the vestibule is connected to the constriction zone. A vestibule may also be referred to as a “goblet.” The vestibule and the constriction zone together define the tunnel of a nanopore, such as an Msp porin. A “constriction zone” or the “readhead” refers to the narrowest portion of the tunnel of a nanopore, such as an Msp porin, in terms of diameter, that is connected to the vestibule. The length of the constriction zone may range from about 0.3 nm to about 2 nm. Optionally, the length is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, or 3 nm, or any range derivable therein. The diameter of the constriction zone may range from about 0.3 nm to about 2 nm. Optionally, the diameter is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, or 3 nm, or any range derivable therein. A “tunnel” refers to the central, empty portion of a nanopore, such as an Msp porin, that is defined by the vestibule and the constriction zone, through which a gas, liquid, ion, or analyte (e.g., a polymer, such as a polynucleotide) may pass. A tunnel is an example of an opening of a nanopore.

Various conditions such as light and the liquid medium that contacts a nanopore, including its pH, buffer composition, detergent composition, and temperature, may affect the behavior of the nanopore, particularly with respect to its conductance through the tunnel as well as the movement of a polymer (e.g., polynucleotide) with respect to the tunnel, either temporarily or permanently.

In some examples, the disclosed system for nanopore sequencing comprises a nanopore, such as an Msp porin, having a vestibule and a constriction zone that define a tunnel, wherein the tunnel is positioned between a first liquid medium and a second liquid medium, wherein at least one liquid medium comprises a polymer (e.g., polynucleotide), and wherein the system is operative to detect a property of the polymer (e.g., a sequence of the polynucleotide). The system may be operative to detect a property of a polymer (e.g., polynucleotide) comprising subjecting a nanopore, such as an Msp porin, to an electric field such that the polymer interacts with the nanopore (e.g., Msp porin). The system may be operative to detect a property of the polymer comprising subjecting the nanopore (e.g., Msp porin) to an electric field such that the polymer electrophoretically translocates through the tunnel of the nanopore (e.g., Msp porin). In some examples, the system comprises a nanopore (e.g., an Msp porin) having a vestibule and a constriction zone that define a tunnel, wherein the tunnel is positioned in a lipid bilayer between a first liquid medium and a second liquid medium, and wherein the only point of liquid communication between the first and second liquid media occurs in the tunnel. Moreover, any nanopore (e.g., Msp porin) described herein may be comprised in any system described herein. In some examples, the system may further comprise an amplifier or a data acquisition device. The system may further comprise one or more temperature regulating devices in communication with the first liquid medium, the second liquid medium, or both. The system described herein may be operative to translocate a polymer through an aperture of a nanopore (e.g., through an Msp porin tunnel) either electrophoretically or otherwise.

As used herein, “adjacent” means next to or adjoining. In some instances, a reporter element may be referred to as being “adjacent to” another element such as an arresting construct. Adjacent in the context of an element (e.g. a structure, a moiety, or otherwise) of a molecule may be used to mean these elements are covalently bound to one another either directly or through some intermediary structure.

The aspects and examples set forth herein and recited in the claims can be understood in view of the above definitions.

### Overview of Nanopore Sequencing, and Cleavable Cyclic Loop Nucleotides for Use in Nanopore Sequencing

A common drawback with nanopore sequencers is that the nanopore is sensitive to multiple bases of a polynucleotide (such as DNA) strand in the nanopore, as opposed to reading single base one at a time. For example, the MspA nanopore has a constriction region which serves as a readhead of at least 4 nucleotides (termed a “k-mer”), resulting in minimally 256 (4\hat{ }4) different permutations of 4-mer sequences that needs to be deconvolved. For a k-mer of 5 bases, the number of possible signals is 4\hat{ }5=1,024. A longer readhead will result in an exponential increase in number of signals to be differentiated, which complicates the sequencing readout and increases the complexity of base calling, thus reducing accuracy. Another issue with nanopore sequencers is that the speed of translocation of natural single stranded DNA is in the order of >10 million nucleotides per second, way above the rate that is compatible with electronics and detectors.

As provided herein, in some examples, by using cleavable sites along the polynucleotide (e.g., DNA) backbone while conjoining adjacent nucleobases with a barcoding region (reporter moiety), the disclosed technology allows the distance between adjacent nucleobases to be increased and negates the need to deconvolve a large number of signals. Once the backbone is cleaved at a certain location to open the cyclic loop containing a barcode region, the polynucleotide is elongated with the barcode region connecting adjacent nucleobases. The barcode region of an elongated polynucleotide may contain one or more reporter moieties, which can occupy the entire readhead of the nanopore for highly accurate single molecule sequencing with single base resolution.

In some examples, the disclosed technology allows having 1 nucleobase of the elongated polynucleotide to reside in the readhead at any point in time, successfully reducing the diversity of reads to 4 (A, T (or U), C and G), enabling more accurate sequencing at a lower cost.

In addition, a tick mark may be provided in addition to the reporter(s) in order to provide an unique signal that may be differentiated from the signals of reporters associated with various nucleobases. The tick mark may allow detections of the occurrence of a mismatch, deletion, or other reading error. In some examples, an arresting construct may be positioned adjacent to a reporter and/or a tick mark in order to provide a mechanism by which the polynucleotide may be ratcheted through or slowly translocate through the nanopore with relatively high fidelity. In further examples an arresting construct may be positioned in two locations, after the tick mark and after the nucleobase reporter. This may operate as a failsafe to inhibit or prevent errors that may have resulted in the ratcheting of the polynucleotide sequence. The disclosed technology may provide various advantages over the prior art including relatively high throughput, cheaper and more accurate DNA sequencing.

Example systems and methods for performing nanopore sequencing now will be described in greater detail.

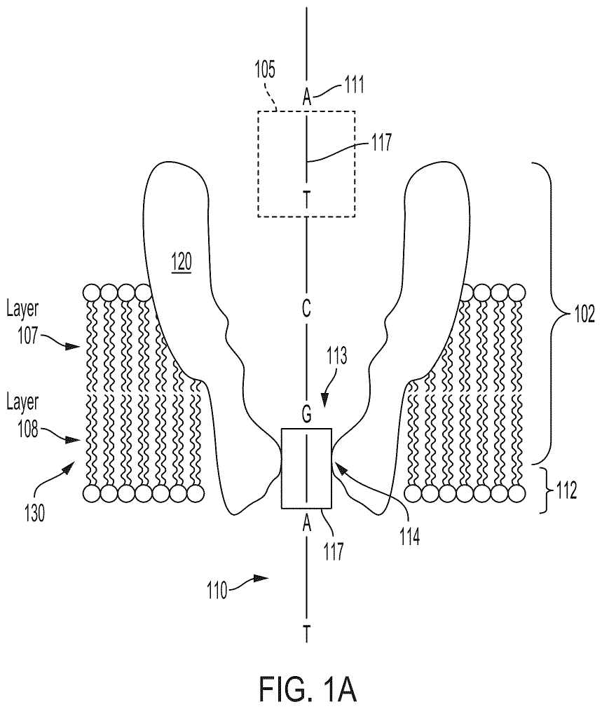

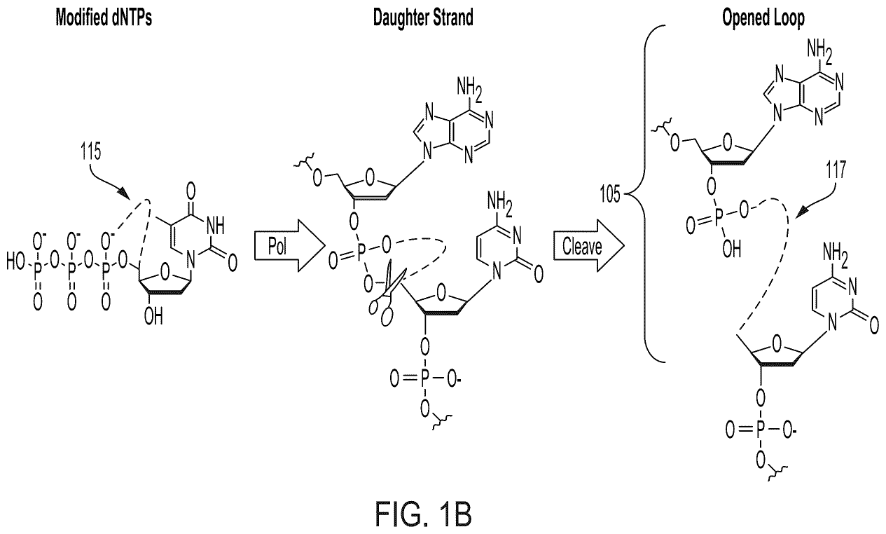

FIGS. 1A and 1B schematically illustrate an example of sequencing an elongated polynucleotide and the elongation of a modified nucleotide with a cyclic loop. Referring first to FIG. 1A, a nanopore (e.g., protein nanopore) 120 is disposed through a membrane (barrier) 130. An elongated polymer (e.g., polynucleotide) 110, which may encode a sequence of a polynucleotide, translocates through the nanopore 120, for example responsive to a voltage bias applied across membrane 130. The polymer 110 includes a sequence of monomer units 105, each of which encodes an identity of a nucleotide in the polynucleotide. In a manner such as will be described in greater detail below, each monomer unit 105 may include one or more reporter moieties and one or more arresting constructs (ARCs). In some examples, the monomer units 105 may include cyclic loop modification regions 117 between successive nucleotides 111, although such modification regions may not necessarily be cyclic at the specific time illustrated in FIG. 1A (e.g., when monomer units 105 are coupled to one another to form polymer 110). Through the use of monomer units 105 (e.g., by introducing a cyclic loop modification 117), the k-mer length—that is, the number of reporter moieties within the readhead of the nanopore—can be reduced to 1, resulting in just 4 signals (for A, T, C and G) and reducing the complexity of base calling. A characteristic reporter/barcode may be assigned to each of the 4 individual bases to achieve base recognition. In some examples, the monomer unit 105 (which also may be referred to as a signal unit or moiety) includes a nucleotide (e.g., a “T” nucleotide) and a corresponding cyclic loop modification 117, which may contain a reporter that serves as the barcode for that nucleotide (e.g., for T). Diversity of reads is reduced to 4 with a single reporter moiety (e.g., barcode) which is characteristic of each nucleobase residing in the nanopore readhead at a given time.

In some examples, to determine the sequence of a polynucleotide (illustratively, DNA), a daughter strand of the polynucleotide is prepared using monomer units 105 that encode nucleotides of the polynucleotide. In the nonlimiting example illustrated in FIG. 1B, the monomer units include modified nucleotides that include a cyclic loop 115. Each type of nucleotide has a cyclic loop 115 that has one or more encoding regions that allow the identity of the associated nucleotide to be identified in a nanopore sequencer. Such encoding regions may be referred to herein as “cyclic” because encoding regions form, or are part of, a loop (heterocycle) at times before the modified nucleotides are coupled to one another to form the polymer, even though the encoding regions may no longer be cyclic at times after the modified nucleotides are coupled to one another to form the polymer.

FIG. 1B schematically shows an example of the formation of the elongated polymer (e.g., polynucleotide) 110 as depicted in FIG. 1A. As shown in FIG. 1B, modified nucleotides are used for (e.g., provided for) forming a daughter strand of the polynucleotide (polynucleotide not specifically illustrated). In this example, each of the modified nucleotides (e.g., modified dNTPs) has a cyclic loop structure 115. Using the polynucleotide as a template, polymerase (Pol) synthesizes a daughter strand by coupling the modified nucleotides to one another in an order that encodes the sequence of the polynucleotide. Thus, in some examples, the prepared daughter strand would incorporate the cyclic loop nucleotides. As illustrated in FIG. 1B, the daughter strand can then be cleaved at locations on the backbone, which opens up the cyclic loops and creates an elongated polymer (which may be referred to as a polynucleotide) that includes a sequence of monomer units 105 coupled to one another. For example, as illustrated in FIG. 1B, monomer units 105 include respective cyclic loop modifications 117 linking and spacing out the adjacent nucleotides. In some examples, the cyclic loop modifications on the modified dNTPs become the cyclic loop modifications 206 in the elongated polymer (e.g., polynucleotide) that create distance between adjacent nucleotides.

In some examples, each of the monomer units 105 may include a first reporter moiety, a second reporter moiety, a first arresting construct, and a second arresting construct. In some examples, each of the monomer units 105 may include a first reporter moiety, a second reporter moiety having a different signal (e.g., electrical) characteristic than the first reporter moiety, and at least one arresting construct (e.g., a first arresting construct, and optionally a second arresting construct). In the particular example shown in FIGS. 1A-1B, every nucleotide of the elongated polynucleotide 110 is attached to a cyclic loop modification 117. In some examples, the cyclic loop modification 117 may comprise at least one reporter that encodes the nucleotide, at least one arresting construct, and a tick mark reporter. While not being bound to any specific theory, the arresting construct may pause (e.g., stop or slow) the polymer translocation, so the encoding regions(s) in the cyclic loop modification may be allowed to spend more time at the readhead during sequencing.

In some examples, the present disclosure is related also to derivations of the cyclic loop to reduce the occurrence of read errors and improve readout accuracy. In some examples, the present disclosure is related to ways in which the cyclic loops can be adjusted to enhance and/or fine-tune their readout properties. In some examples, the present disclosure is related to a system for determining a sequence of a polynucleotide using the method disclosed herein. In some examples, a constant stimulus (e.g., no change in voltage bias) is required to advance a nucleotide through the readhead of a nanopore. In some examples, a change in voltage bias is implemented to advance a nucleotide through the readhead of a nanopore.

As noted above, after the polymerization process is completed, cleavage at predetermined locations opens the loops and increases the distances between adjacent nucleotides. Cleavage of the daughter strand can be designed to occur at any part of the backbone of the daughter strand as long as such cleavage occurs within the loop structure between the two positions where the cyclic loop is attached to adjacent nucleotides in the structure. Cleavage of the daughter strand along the backbone opens the loops and elongates the daughter strand, leaving a modification (which may be referred to as a cyclic loop modification, although not cyclic) conjoining the backbone phosphate and the sugar. In examples where the cyclic loop modification contains an arresting construct configured to interact with the nanopore, the arresting construct may be used to pause (e.g., slow or halt) the translocation of the elongated polymer through the nanopore and allow the nucleotides or one or more reporter moieties in the cyclic loop modification to be read by the nanopore one at a time.

In some examples where a reporter moiety (such as a reporter barcode) is a part of the monomer units making up the daughter strand (e.g., the monomer units include a cyclic loop modification), the cleaved product, i.e., the elongated polymer 110, exposes a series of reporter moieties which encode (report) the identities of bases to which they correspond. In examples where the cyclic loop modification also contains an arresting construct configured to interact with the nanopore, the elongated polymer can be sequenced in the nanopore one barcode at a time.

### Example Operations

The polynucleotide sequencing techniques described herein utilize a nanopore, which can provide a path for an ionic current when a bias is applied across the nanopore. For example, as the polymer 110 translocates through the nanopore, such translocation may result in a time-varying sequence of unique ionic current blockades at the nanopore and, therefore, a time-varying sequence of unique nanopore resistances depending on the identity of the nucleotides/reporters or the sequence combination of nucleotides/reporters. By measuring the ionic current and/or the nanopore resistance, the nucleotide or the sequence combination of nucleotides at or near the nanopore can be identified. In other words, the polymer translocating relative to the nanopore may modulate the electrical properties of the nanopore such that the nucleobase sequence of the polynucleotide encoded by the polymer can be identified. For example, the ionic current through the nanopore or the electrical resistance at the nanopore may be a function of the reporter or reporter moiety at or near the nanopore.

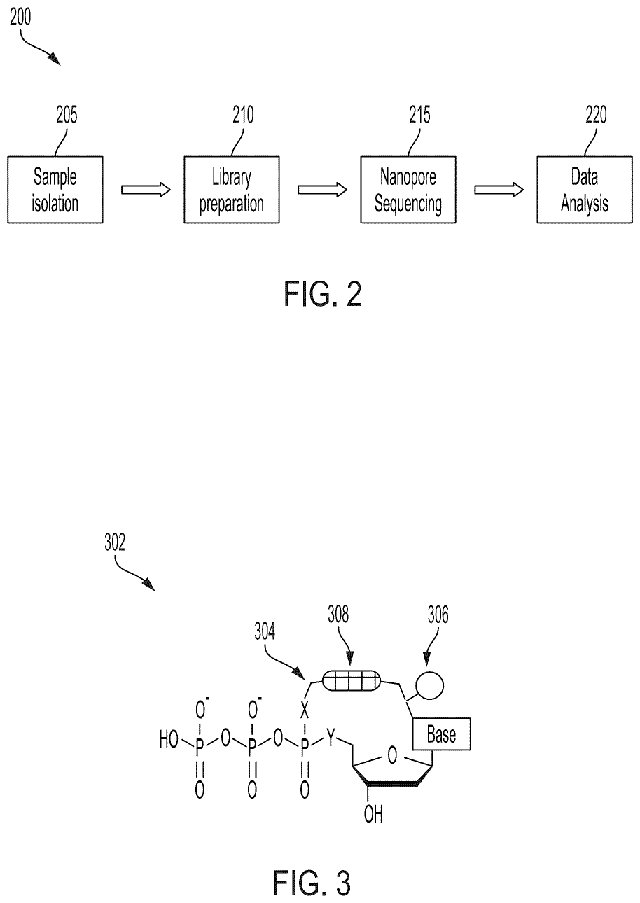

FIG. 2 illustrates an example workflow 200 for the nanopore sequencing method described herein. The workflow 200 starts at step 205, isolation of sample polynucleotide (such as DNA) from a biological source using any suitable extraction method(s). After isolation of sample polynucleotide, the workflow moves to step 210, where the sample polynucleotide is subjected to a library preparation process which comprises using the sample polynucleotide as a template for synthesizing a new daughter strand, for example using modified polynucleotides with cyclic loop modifications. In some examples, the cyclic loop structure comprises at least one arresting construct, one or more reporters, and a tick mark. In various examples, the cyclic loop bridges the nucleobase and the phosphate group. However, a person having ordinary skill in the art would appreciate that in other examples the cyclic loop could be configured to span any number of nucleobases (i.e., 1, 2, 3, 4, 5, etc). The nucleotides may include sites where one or more chemical bonds may be broken to form elongated polymer 110.

After library preparation, the workflow moves to the nanopore sequencing step 215, where the polymer 110 in the library is translocated through the nanopore, and data during translocation is collected to be used for determining the base identities of the sample polynucleotide. After the nanopore sequencing step, the workflow moves to the data analysis step 220, where base calls are made based on the data collected. In some examples, the workflow 200 of FIG. 2 is a part of a sequencing cycle.

Example Cyclic Loop Modifications with Arresting Constructs

FIG. 3 illustrates an example of a modified nucleotide that can be used to form a daughter strand of the sample polynucleotide. The modified nucleotide has a cyclic loop modification 304 appended to the nucleotide 302 at two locations, thereby forming a cyclic loop. The cyclic loop modification further includes an arresting construct 306 configured to slow or modulate the translocation speed of the target polynucleotide through a nanopore readhead. The cyclic loop modification further includes a reporter element 308 encoding the nucleobase that the reporter element attaches to or is associated with. Each reporter element 308 produces a specific signal when traversing the nanopore. In some examples, the reporter element 308 for each type of nucleobase produces a substantially different signal than the reporter elements for other types of nucleobases. These signals are differentiated within the range of signals (voltages, currents, optical signals, etc.) used in the method disclosed herein.

As described herein, a modified nucleotide (e.g., cyclic loop nucleotide) includes a cyclic loop modification bridging a nucleobase and a phosphate group of the nucleotide. In some examples, the cyclic loop modification includes a reporter encoding the identity of the nucleobase, an arresting construct adjacent to the reporter, and a tick mark. In some examples a second arresting construct may be located adjacent to the tick mark. In some examples, one or more spacer regions may also be included in the cyclic loop modification. The tick mark may also be a reporter, but in some examples, the tick mark is not used for identification of the nucleobase. The tick mark may provide (generate) a signal that is different from that of any of the reporters associated with each of the nucleotides. In other examples, the tick mark may provide a signal that is unique to the nucleobase.

In some examples, the cyclic loop nucleotide can have one of the following structures:

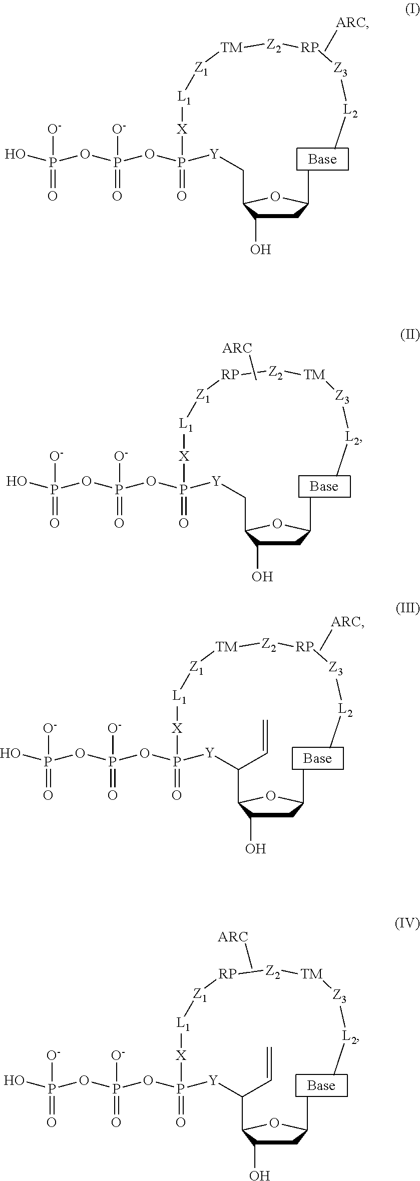

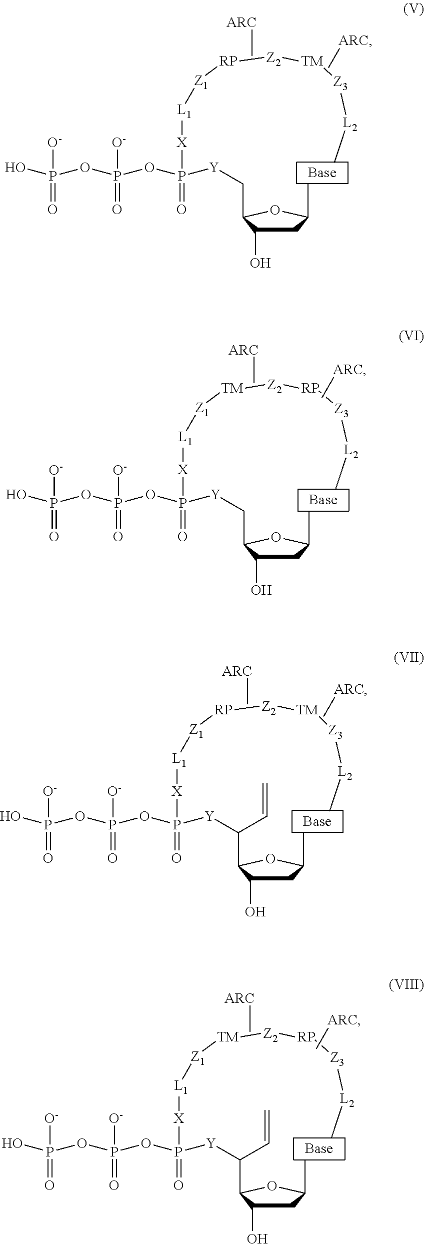

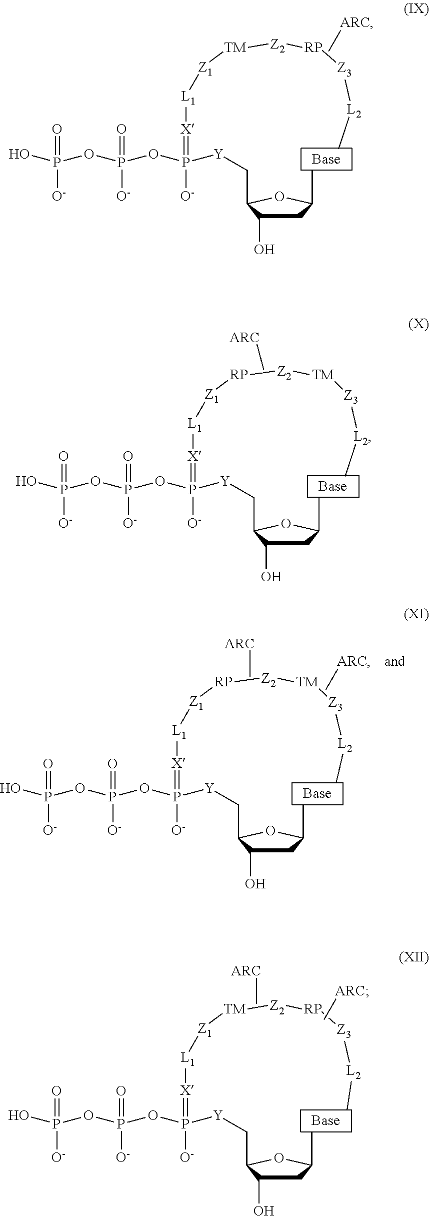

wherein: X can be —O—, —CH₂—, —NSO₂—, —NH—,

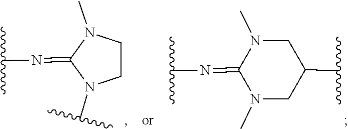

X′ can be ═N—SO₂—; ═NH—CO—, or

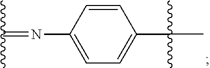 [SMILES: *c1ccc(N=*)cc1]

Y can be —O—, —S—, —NH—, or —Se—; Base is the nucleobase; L₁ and L₂ are linking groups or absent; Z₁, Z₂, and Z₃ are spacers or absent; RP is a reporter encoding the nucleobase; TM is a tick mark; and each ARC is an arresting construct.

In some examples having the structures I-XII, one or more of the X′, X, spacers, or linking groups may be absent. However, such structures can be helpful to provide spacing between reporter elements, the Base, and/or tick marks to improve the fidelity of the readout. In some examples the tick marks, reporters, X′, X, spacers, or linking groups may include at least two or more, at least three or more, at least four or more, at least five or more, or at least six or more subunits, such as phosphate units, peptide units, polyamide, and synthetic polymer units described elsewhere herein.

In some examples having the structures I-XII, the cyclic loop modification may consist of the subunits shown in structures I-XII. In some examples, the subunits of the cyclic loop modification are directly adjacent to each other and sequentially configured. In some examples, the linker groups may be replaced with spacers and the spacers may be replaced with linker groups. In some examples the reporter is a partial encoding of the modified nucleotide, such that a sequence of reporters encodes each nucleotide (or polynucleotide) using a modified cyclic loop.

In some examples, the arresting construct is a branch off of the “loop” of the modified nucleotide (e.g. creating a branched polymer). In some examples, the width of the arresting construct is larger than the width of the linker, spacer, reporter, and/or tick mark. In some examples, the width of the arresting construct is at least 20%, at least about 30%, at least about 40%, at least about 50%, at least about 60%, at least about 75%, or at least about 100% larger than the width of the linker, spacer, reporter, and/or tick mark.

Some examples may have cyclic loops with varying configurations. For example, in some examples, the cyclic loop modification may have one or more linking groups flanking the ends of the cyclic loop, such as to attach the cyclic loop to the nucleotide. In some examples, the attachment construct (for example X) may be configured to attach through a single bond to a phosphate of the phosphate backbone of the nucleotide. Such attachment construct may, in some examples, comprise an ether linkage, a methyl linkage, a sulfonamide linkage, an amine linkage, a linkage comprising a five or six-membered heterocyclic ring, an imidazole-2-imine linkage, an N,1,3-trimethylimidazolidin-2-imine linkage, a pyrimidin-2 (1H)-imine linkage, or a tetrahydropyrimidin-2 (1H)-imine linkage. In some examples, the attachment construct (for example X′) may be configured to attach through a double bond to a phosphate of the phosphate backbone of the nucleotide. Such attachment construct may, in some examples, comprise a sulfonamide linkage, an amide linkage, an imine linkage to a five or six membered heterocycle or homocycle, a tolylmethanimine linkage, or a N-p-tolylmethanimine linkage.

In some examples, the cyclic loop may include one or more linker groups, one or more spacers, one or more reporters, one or more tick marks, and/or one or more arresting constructs. In some examples, the cyclic loop comprises two or more linker groups, two or more spacers, one or more arresting constructs, and two or more reporters or tick marks. In some examples, each linker group is adjacent to a spacer. In some examples, each reporter is adjacent to an arresting construct. In some examples, each tick mark is adjacent to an arresting construct. In some examples, each spacer is adjacent to an arresting construct. In some examples, the cyclic loop has a reporter adjacent to an arresting construct, and a tick mark adjacent to an arresting construct, and a spacer is between the reporter and the tick mark. In some examples, the cyclic loop may contain a reporter and a tick mark with a single arresting construct adjacent to the reporter. In some examples, the cyclic loop may comprise a spacer followed by a tick mark followed by a spacer followed by a reporter, and in some examples, those units are sequentially or directly adjacent to each other. In some examples, the cyclic loop may comprise a spacer followed by a tick mark followed by an arresting construct followed by a spacer followed by a reporter, and in some examples, those units are sequentially or directly adjacent to each other. In some examples, the cyclic loop may comprise a spacer followed by a tick mark or a reporter followed by an arresting construct followed by a spacer followed by a reporter or tick mark followed by an arresting construct, and in some examples, those units are sequentially or directly adjacent to each other. In some examples, the cyclic loop can connect to a phosphate in the phosphate backbone through a single bond (i.e. “P—”) or through a double bond (“P═”). In some examples, the cyclic loop can connect to a phosphate of the phosphate backbone through an imine (“—N═”), and the cyclic loop may be connected through either side of the imine group (“═N—”) or (“—N═”). In some examples, the imine may be further connected to a sulfonyl group, a carbonyl group, a methyl group, an amine, an ether, or a heterocycle or homocycle.

In some examples, the cyclic loop can contain two arresting constructs with a spacer and a reporter or spacer between them. In some examples the cyclic loop can contain a repeating subunit of a reporter, an arresting construct, and a spacer, and in some examples the subunit is repeated at least two times. In some examples the cyclic loop can contain one or more repeating subunits of a reporter, a spacer, an arresting construct, a reporter, and a spacer, and in some examples, these units are sequentially adjacent to each other. In some examples, that reporting subunit may be repeated two, three, or four times, or more than four times. In some examples, a linker group is directly attached to the nucleobase of the nucleotide, and in some examples the linker group is sequentially followed by a spacer unit.

In some examples, a vinyl group may be positioned between the ribose or deoxyribose and the phosphate backbone to facilitate the expansion of the cyclic loop or the cleavage between the ribose or deoxyribose and the phosphate backbone. In some examples, the vinyl group may be absent and the ribose or deoxyribose may be connected to the phosphate backbone through an ether, a sulfide, an amine, or a selenium linkage. In some examples where the vinyl group is present, the ribose is connected to a phosphate of the phosphate backbone via an ether linkage.

In some examples, the cyclic loop nucleotide can have one of the following structures:

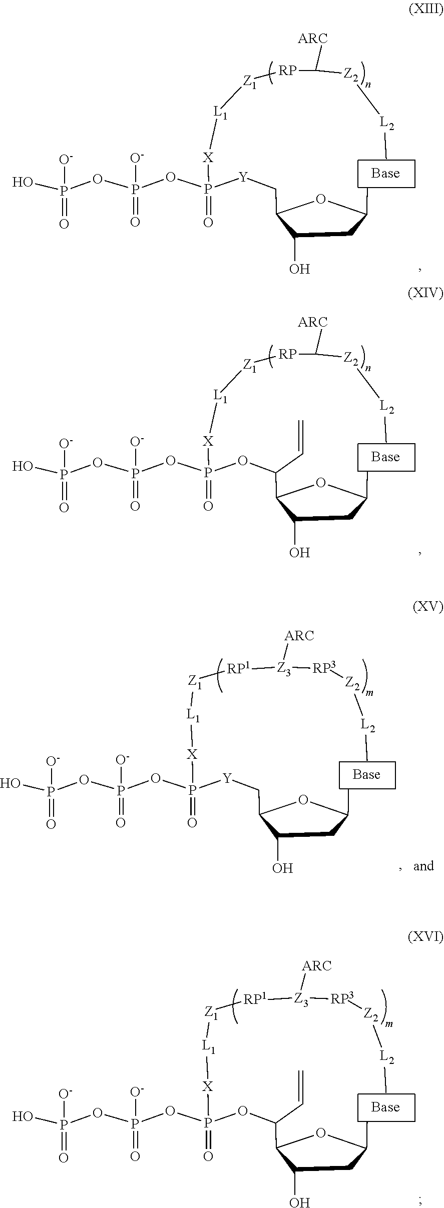

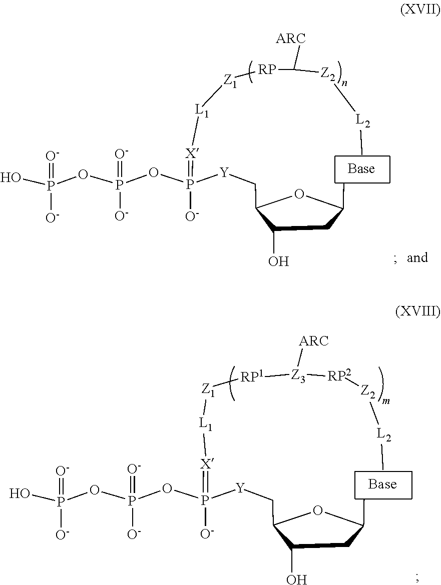

wherein: X is —O—, —CH₂—, —NSO₂—, —NH—,

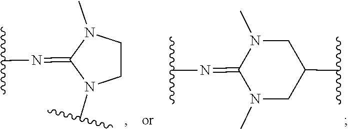

X′ is ═N—SO2-; ═NH—CO—, or

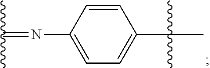 [SMILES: *c1ccc(N=*)cc1]

Y is —O—, —S—, —NH—, or —Se—; m or n is a positive integer; Base is a nucleobase; L₁ and L₂ are each a linking group or absent; Z₁, Z₂, and Z₃ are each a spacer or absent; RP, RP¹ and RP² are each a reporter, wherein a combination of all the RP or a combination of all the RP¹ and RP² encodes the nucleobase; and ARC is an arresting construct.

In some examples having the structures XIII-XVIII, one or more of the X′, X, spacers, or linking groups may be absent. However, such structures can be helpful to provide spacing between reporter elements, the Base, and/or tick marks to improve the fidelity of the readout. In some examples, the tick marks, reporters, X′, X, spacers, or linking groups may include at least two or more, at least three or more, at least four or more, at least five or more, or at least six or more subunits, such as phosphate units, peptide units, polyamide, and synthetic polymer units described elsewhere herein.

In some examples having the structures XIII-XVIII, the cyclic loop modification may consist of the subunits shown in structures XIII-XVIII. In some examples, the subunits of the cyclic loop modification are directly adjacent to teach other and sequentially configured. In some examples, the linker groups may be replaced with spacers and the spacers may be replaced with linker groups. In some examples, the reporter is a partial encoding of the modified nucleotide, such that a sequence of reporters encodes each modified cyclic loop.

In some examples of the structures XIII-XVIII, the attachment construct (for example X) may be configured to attach through a single bond to a phosphate of the phosphate backbone of the nucleotide. In some examples, the attachment construct may comprise an ether linkage, a methyl linkage, a sulfonamide linkage, an amine linkage, a linkage comprising a five or six-membered heterocyclic ring, an imidazole-2-imine linkage, an N, 1,3-trimethylimidazolidin-2-imine linkage, a pyrimidin-2 (1H)-imine linkage, or a tetrahydropyrimidin-2 (1H)-imine linkage. In some examples, the attachment construct (for example X′) may be configured to attach through a double bond to a phosphate of the phosphate backbone of the nucleotide. In some examples, the attachment construct may comprise a sulfonamide linkage, an amide linkage, an imine linkage to a five or six membered heterocycle or homocycle, a tolylmethanimine linkage, or a N-p-tolylmethanimine linkage.

In some examples, the arresting construct is a branch off of the loop of the modified (looped) nucleotide (e.g. creating a branched polymer). In some examples the width of the arresting construct is larger than the width of the linker, spacer, reporter, and/or tick mark. In some examples, the width of the arresting construct is at least 20%, at least about 30%, at least about 40%, at least about 50%, at least about 60%, at least about 75%, or at least about 100% larger than the width of the linker, spacer, reporter, and/or tick mark.

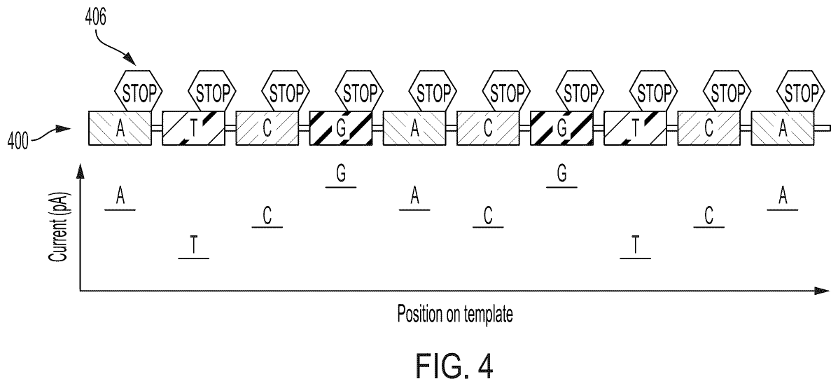

FIG. 4 illustrates a generic elongated polymer 400 comprising arresting constructs and reporter elements, wherein each reporter element produces a distinguishable signal when traversing a nanopore. In some examples, cyclic loop modifications are incorporated into a daughter strand of the polynucleotide, that is, into polymer 400, in a manner such as described with reference to FIGS. 1A-1B. In some examples, each cyclic loop nucleotide contains a unique barcoding/reporter region (i.e., reporter element) that is specific to a corresponding one of the original bases (e.g., A, T (or U), C, or G). The daughter strand is then “elongated” by cutting the cleavable sites on the polynucleotide backbone. Consequently, when sequencing the daughter strand, the nanopore can “read” the barcoding/reporter region to generate a signal that can be used to identify the base that such barcoding/reporter region encodes. The reporter element that is introduced in the daughter strand via polymerization is designed to occupy the readhead of the nanopore entirely, and is spaced apart from other reporter elements, for example with a spacer that increases the distance between the adjacent reporter elements, hence reducing the number of signals to just four, i.e., one per nucleobase. Thus, these modifications allow barcode-based decoding of individual bases.

In some examples, an arresting construct may be present to modulate translocation speed of the polymer 400. In some examples, the cyclic loop may contain at least one non-barcoding spacer which allow the daughter strand to elongate after cutting the cleavable sites. The non-barcoding spacers may produce a distinguishable signal or a distinguishable signal break from signals of the nucleobases when passing through the nanopore, thereby isolating and/or enhancing the recorded signals from the nucleobases. In some examples, the spacer may contain both barcoding/reporter regions and non-barcoding spacers. In some examples, the spacer may be the barcoding/reporter element.

### Example Tick Marks

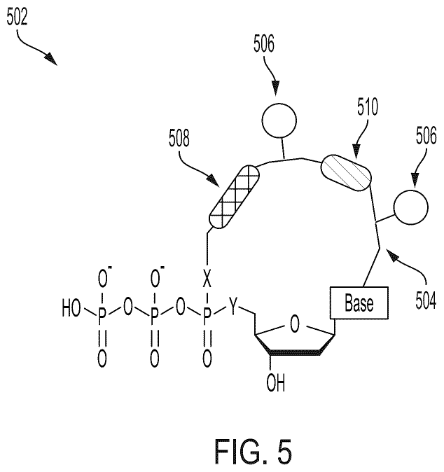

In some examples, the monomer units of polymer 110 (including, e.g., the cyclic loop modification) can further include a tick mark, which produces a signal that is different from those produced by the reporters associated with, and uniquely encoding, all nucleobases. FIG. 5 illustrates another example of a modified nucleotide that may be used to form a daughter strand of a sample polynucleotide in a manner such as described with reference to FIGS. 1A-1B. The example modified nucleotide 502 illustrated in FIG. 5 has a cyclic loop modification 504 appended to the nucleotide 502 at two locations, thereby forming a cyclic loop. The cyclic loop modification 504 further includes one or more arresting constructs 506 configured to slow or modulate the translocation speed of the target polynucleotide through a nanopore readhead. The cyclic loop modification 504 further includes one or more reporter elements 508 encoding the nucleobase. Each reporter element 508 produces a specific signal when traversing the nanopore, and the reporter element(s) 508 for each nucleobase produces substantially different signals.

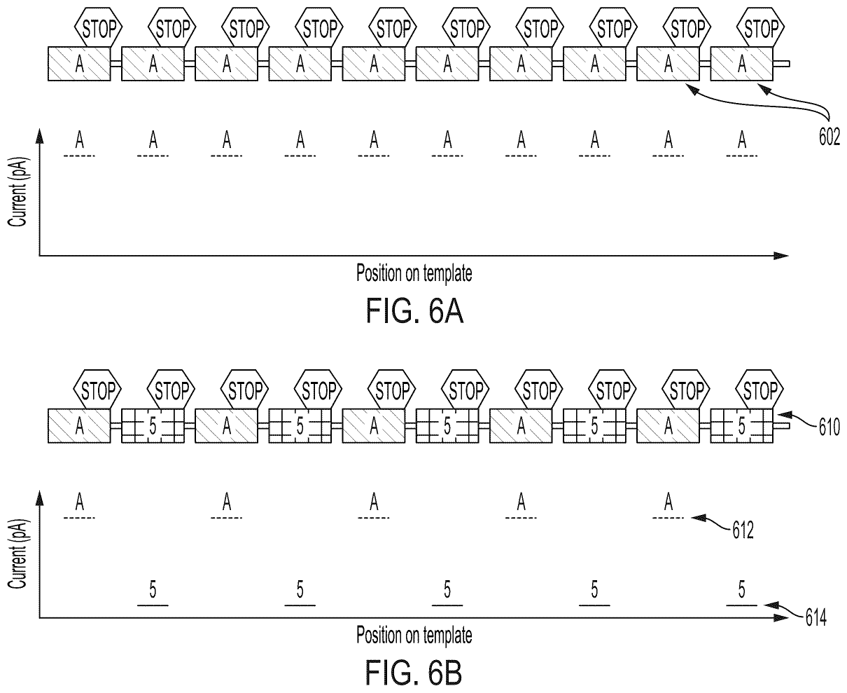

The improved modified nucleotide illustrated in FIG. 5 additionally includes a tick mark 510, which encodes a signal that is unique from the signal of the nucleobase reporters 508. For example, each nucleobase may contain one or more reporters 508 that generate a respective signal for A, T (or U), C, or G, and the tick mark 510 contains a signal labeled as “5” In FIG. 6B. In some examples, the tick mark 510 provides a signal that is decipherable from the nucleobase reporter 508 signal, thus analytically different enough to distinguish the two. In other examples, the tick mark signal may differ substantially from the nucleobase reporter 508 signal. Thus, the modified nucleobase 502 may include two unique signals (a reporter 508 signal and a tick mark 510 signal) that may be differentiated from each other to improve the sequencing resolution.

In the nonlimiting example shown in FIG. 5, the cyclic loop contains two arresting constructs 506. One arresting construct 506 is position adjacent to the reporter element 508, and one arresting construct 506 is adjacent to the tick mark 510. In some examples, one arresting construct 506 is between the reporter element 508 and the phosphate group, and another arresting construct 506 is between the reporter element 508 and the tick mark 510. Thus, the sequencing operation may be slowed or stopped first by one of the arresting constructs 506 to read out the reporter element 508, and then slowed or stopped by another one of the arresting constructs to read out the tick mark 510. In this example, providing two arresting constructs 506 in the cyclic loop may ensure that each of the reporter element 508 and tick mark 510 are not skipped, which reduces the error resulting from mismatch errors, deletion errors, or the like. In other examples such as described elsewhere herein, any suitable number of arresting constructs may be used to pause translocation of respective reporters (such as tick marks) through the aperture of a nanopore, so as to facilitate detection of signals from such reporters. Nonlimiting examples of arresting construct modifications are discussed with regard to FIGS. 6A and 6B.

In some examples, the cyclic loop may further include one or more spacer regions which operates to increase the distances between successive reporting elements 508 and/or tick marks 510. In the case where a voltage pulse is applied, the spacer(s) permits the force on the arresting construct due to the voltage pulse to fade away before the next arresting construct arrives in the pore to inhibit or avoid skipping reporters. In the case a constant voltage bias is applied, the translocation of the polymer (i.e. in “auto ratchet” mode) does not rely on a voltage pulse or other stimulus for translocation, but rather automatically advances at a relatively slow rate during the application of a constant voltage. In the auto ratchet mode, the spacer also provides a means to control translocation rates and distance the reporters.

As shown in FIGS. 6A and 6B, the incorporation of a reporter element 508 and tick mark 510 allows the fidelity of the sequencing readout to be optimized. For example, FIG. 6A shows the readout of an example polynucleotide where the polynucleotide 602 consists of a single homopolymer sequence segment, illustratively poly-A, in which each nucleotide in the polynucleotide includes a reporter moiety (rectangle denoted “A”) and an arresting construct (“stop” sign), but lacks a tick mark. For example, when the homopolymer sequence consists of repeating “A” nucleobases, the current readout only provides a single, consistent signal, which can introduce uncertainty or errors in the gathered signal even as the polymer advances between successive reporter positions. The measured signal would not directly indicate that the polynucleotide 602 has advanced from one nucleobase to the next resulting in be uncertainty in the length of the homopolymer segment. Even with arresting constructs that move as desired 90% to 95% of the time, which is a realistic expectation for efficiency, the readout may eventually have a base error rate in homopolymers of 5% to 10%.

Incorporating a tick mark 610 (rectangle denoted “5”), as shown in FIG. 6B, reduces the uncertainty provided in the signal that otherwise may result from homopolymer sequences. For example, the tick mark 610 provides a unique signal that can be differentiated from the signals from the reporters of the nucleobases, thus providing a fingerprint indicating that translocation has occurred. For example, FIG. 6B shows the tick mark 610 provides a tick mark signal 614 that is unique and separable from the nucleobase reporter signal 612, producing a readout of A-5-A-5-A-5-A-5-A-5. Differentiation between these two signals is especially useful in homopolymer stretches where the reporter signals do not change from one nucleotide/reporter to the next. Typically, the nanopore current cannot be monitored during the voltage pulse (the amplifier rails, and translocation otherwise occurs too rapidly in any event), so absent a tick mark there may be no explicit indication that the voltage pulse effectively translocated the arresting construct in a homopolymer region. Additionally, skips or stalls in translocation may not be detected without a tick mark. Particularly, the tick mark can provide redundancy and be an indicator that the nucleotide sequence has advanced through the nanopore, reducing the error that results from uncertainty in the translocation of the sequence. In addition to improving the readout of homopolymer regions, the presence of a tick mark may aid the accuracy of non-homopolymer sequences. Advantageously, the use of tick mark reduces the nucleobase readout error rate by a factor of about 10-fold. This is an unexpectedly large improvement in the accuracy of the readout of the polynucleotide sequence.

### Error Correcting Nucleotides

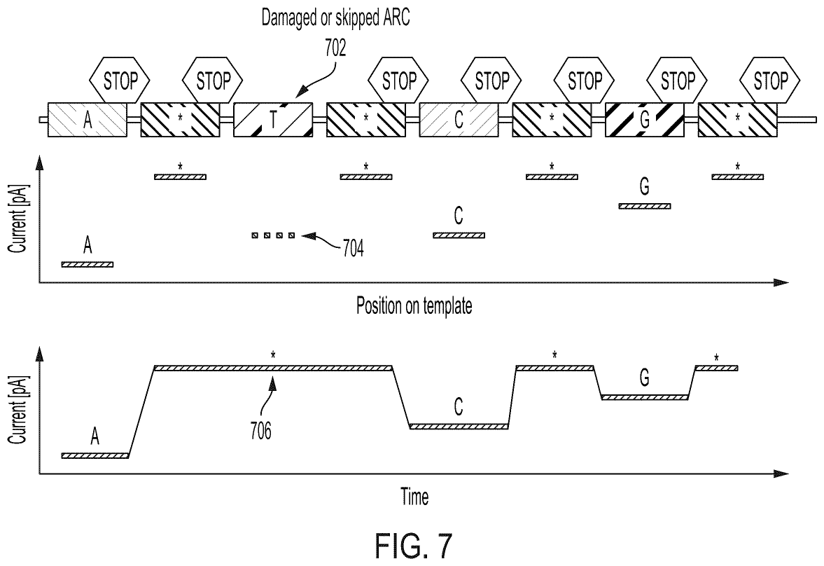

As discussed herein, a tick mark may be incorporated into a cyclic loop in order to provide an indication of advancement of the polynucleotide sequence through the nanopore, especially where a homopolymer sequence is present. In some examples, the each nucleotide in the polynucleotide sequence may be provided with a single unique tick mark to generate a tick mark signal, such as a “5.” This “5” does not uniquely represent the identity of the nucleobase, but rather indicates that there is an advancement of the nucleobase. In other words, the tick mark may not carry any information associated with the nucleobase other than an indication of advancement of one nucleobase to another. In examples such as described above with reference to FIG. 5, the cyclic loop may include (be provided with) two arresting constructs, where one arresting construct is between the tick mark and the reporter for the nucleobase. However, the use of a single tick mark signal and two arresting constructs can lower the speed at which translocation, and ultimately sequencing, can occur. Further, unexpected errors may occur in readout with a single tick mark signal (denoted by * in the rectangle), which is shown in FIG. 7.

FIG. 7 demonstrates an example sequence associated with the modified nucleobase in FIG. 5 and potential errors associated therewith. More specifically, FIG. 7 depicts an example of an error that may occur where each tick mark is only encoded with a single signal (denoted by the positions with a * in the rectangle). In this example, an arresting construct associated with the nucleobase T has a faulty arresting construct 702 because the arresting construct is damaged, or because the arresting construct is otherwise skipped. As a result, the sequence, which is A-T-C-G as shown in the upper portion of FIG. 7, is read as A-C-G because the voltage signal 706 could not differentiate between the signals of the two tick marks that surround the T nucleobase. Thus, a single tick mark signal cannot differentiate this single missing signal 704. This type of error may occur where there is a heterogeneous sequence, as illustrated, or this type of error can also occur where there is a homopolymer sequence, as discussed with regards to FIGS. 6A-6B.

Therefore, it may be desirable in examples disclosed herein to improve upon the tick mark signal and provide one or more unique tick mark signals that are distinct from each other. The unique tick mark signals can be configured to be unique from the signals of the nucleobase reporters as well.

In certain examples, the cyclic loop may be an Error-Correcting (EC) nucleotide. An error correcting nucleotide is generally a modification of a cyclic loop that provides at least two distinct signals or orders of signals associated with each nucleobase. For example, an EC nucleotide may have a reporter region associated with the nucleobase “A” and a tick mark signal only associated with the nucleobase “A.” By having two distinct signals or orders of signals associated with each nucleobase, a readout error, such as that shown in FIG. 7 can be avoided. The reporters of EC nucleotides may be referred to as EC reporters.

In some examples, the EC nucleotides of the present disclosure are advantageous as they more clearly resolve transitions between bases, compared to using tick marks alone or simply using reporter constructs (such as non-error correcting constructs). The EC nucleotides of the present disclosure are advantageous in that they are capable of resolving homopolymer regions with relatively high fidelity. Additionally, some EC nucleotides (such as F-2-8 nucleotides, described further in Table 1 below and elsewhere herein) lower basal deletion error rates by 2.5-fold (or more) as compared to a tick-mark reporter (such as in FIG. 5), and by about 10-fold to about 25-fold more than having no tick mark reporter or transition marker at all. The F-2-8 EC nucleotides are advantageous in that they incur no additional penalty to the net sequencing rate as compared to having only a tick-mark reporter (i.e. also known as an F-2-5 nucleotide, described further in Table 1 below and elsewhere herein). Additionally, the EC nucleotides can lower the requirements for the instrument signal to noise ratio (SNR) by imposing an alternating pattern of high/low signals. Moreover, the EC nucleotides can provide insertion error tolerance and mismatch error tolerance and allow for faster net sequencing rates due to improved error tolerance. Finally, the EC nucleotides may lower the requirements for numbers of distinguishable current levels and can provide information that an error has occurred, and the type of error (e.g. single deletion, mismatch error, etc.) (e.g. such as in an F-2-4 nucleotide, described further in Table 1 below and elsewhere herein).

The EC nucleotides of the present specification can be used with any of the nanopores described in this disclosure and are readily implemented as an extension of existing technology. The EC nucleotides may be highly relevant for improving the nanopore sequencing accuracy, especially for ratchet-like approaches.

One property consistent with the EC nucleotides disclosed herein is that each individual nucleobase (A, T (or U), C, or G) may be encoded with more than one reporter current. In comparison to other example approaches described herein, such as a tick mark approach with a single tick mark signal and four unique reporters, or the raw code approach with only a single reporter, the general case of EC nucleotides does not require a one-to-one correspondence between a unique current level and an encoded base's identity. For example, each of the monomer units forming polymer 110 may include a sequence of EC reporter signals (e.g., the nucleobase may be provided with a sequence of EC reporter signals in the cyclic loop), and in some examples the number of unique EC reporter signals may be fewer than the number of nucleobases (4).

The complexity of the combination of EC reporter signals (EC codes) may be described through an EC naming convention having a letter followed by two numbers (i.e. X-#-#). The letter may indicate whether the nucleotide sequence is compatible with forward translocation only (F) or with forward and backward translocation through alternating forward and reverse currents (bipolar, B). The first (middle) number indicates the length of the encoding (i.e. the number of reporters in each monomer unit, e.g., cyclic loop) in that example, and the last (right-most) number indicates the number of possible unique current signatures in that example. Applying this naming convention to a “raw code” scheme (such as that represented in FIG. 3), in this scheme each reporter has one reporter region and there is one unique signal for each of the bases (4). Thus, the EC naming code for raw coding is F-1-4 (forward, one reporter per nucleobase, and 4 unique current signals). If this naming convention is applied to a “tick marks” scheme (such as those represented in FIG. 5) the EC naming would be F-2-5, as the strand is designed for forward translocation, there are two reporter regions on each cyclic loop, and there are 5 unique current signals (4 for each base plus one unique tick mark signal).

Table 1 below depicts various configurations for EC nucleotides and various parameters associated with each different EC code configuration:

TABLE 1

| | | | | Relative | | Mini- |
|---|---|---|---|---|---|---|
| | | # of | # | time for | Median | mum |
| | | ARCs | unique | single | signal | signal |
| Name | Encoding | required | currents | pass | distance | distance |
| Raw code | A: [0] | 1 | 4 | 1 | 0.42 | 0.25 |
| (F-1-4) | T: [1] | | | | | |
| | C: [2] | | | | | |
| | G: [3] | | | | | |
| Tick mark | A: [0, 1] | 2 | 5 | 2 | 0.5 | 0.2 |
| code | T: [0, 2] | | | | | |
| (F-2-5) | C: [0, 3] | | | | | |
| | G: [0, 4] | | | | | |
| F-2-4 | A: [0, 2] | 2 | 4 | 2 | 0.5 | 0.25 |
| | T: [0, 3] | | | | | |
| | C: [1, 2] | | | | | |
| | G: [1, 3] | | | | | |
| F-2-8 | A: [0, 5] | 2 | 8 | 2 | 0.62 | 0.12 |
| | T: [1, 6] | | | | | |
| | C: [2, 7] | | | | | |
| | G: [3, 4] | | | | | |
| F-3-4 | A: [0, 1, 3] | 3 | 4 | 3 | 0.5 | 0.25 |
| | T: [0, 2, 1] | | | | | |
| | C: [2, 0, 3] | | | | | |
| | G: [2, 3, 1] | | | | | |
| F-4-3 | A: [0, 1, 0, 2] | 4 | 3 | 4 | 0.33 | 0.33 |
| | T: [0, 1, 2, 1] | | | | | |
| | C: [0, 2, 0, 1] | | | | | |
| | G: [0, 2, 1, 2] | | | | | |
| F-4-4 | A: [0, 2, 0, 2] | 4 | 4 | 4 | 0.5 | 0.25 |
| | T: [0, 3, 2, 1] | | | | | |
| | C: [3, 0, 1, 2] | | | | | |
| | G: [3, 1, 3, 1] | | | | | |
| F-5-3 | A: | 5 | 3 | 5 | 0.67 | 0.33 |
| | [0, 1, 2, 0, 2] | | | | | |
| | T: | | | | | |
| | [0, 2, 0, 1, 2] | | | | | |
| | C: | | | | | |
| | [0, 2, 0, 2, 1] | | | | | |
| | G: | | | | | |
| | [0, 2, 1, 0, 2] | | | | | |
| F-5-5 | A: | 5 | 5 | 5 | 0.4 | 0.2 |
| | [1, 4, 0, 2, 3] | | | | | |
| | T: | | | | | |
| | [1, 3, 0, 4, 2] | | | | | |
| | C: | | | | | |
| | [0, 3, 4, 1, 2] | | | | | |
| | G: | | | | | |
| | [0, 1, 3, 2, 4] | | | | | |
| B-6-3 | A: | 3 | 3 | 6 | 0.33 | 0.33 |
| | [0, 1, 0, | | | | | |
| | 1, 0, 1] | | | | | |
| | T: | | | | | |
| | [0, 1, 2, | | | | | |
| | 0, 1, 2] | | | | | |
| | C: | | | | | |
| | [0, 2, 0, | | | | | |
| | 2, 0, 2] | | | | | |
| | G: | | | | | |
| | [0, 2, 1, | | | | | |
| | 0, 2, 1] | | | | | |
| B-6-4 | A: | 3 | 4 | 6 | 0.5 | 0.25 |
| | [0, 2, 3, | | | | | |
| | 1, 2, 1] | | | | | |
| | T: | | | | | |
| | [2, 0, 2, | | | | | |
| | 0, 2, 3] | | | | | |
| | C: | | | | | |
| | [2, 1, 3, | | | | | |
| | 2, 1, 3] | | | | | |
| | G: | | | | | |
| | [0, 1, 0, | | | | | |
| | 3, 0, 3] | | | | | |

As shown above, each EC nucleotide configuration may operate with a varying number of lengths of encoding regions and number of unique signature currents. Interestingly, the number of unique signature currents may be fewer than the number of unique nucleobases that need to be encoded (4). This may be enabled, as shown above, through the order in which the signature currents are read. For example, a cyclic loop with the unique signal order of [0, 1, 3] provides a different signal associated with the nucleobase “A” whereas the unique signal order of [0, 2, 1] associated with the nucleobase “T.”

It should be noted that a person having ordinary skill in the art would comprehend that the sequence of signals listed in Table 1 are purely illustrative, and any sequence of signals may be derived by a person having ordinary skill in the art based upon the application for nanopore sequencing, whether it be optical nanopore sequencing, biological pore nanopore sequencing, or semiconductor nanopore sequencing, sequencing in a microchip, etc. For example, the code for “A” in an F-3-4 scheme may be [0, 1, 3], but a person having skill in the art would apprehend that any designated sequence of 3 signals in an F-3-4 scheme would work as long as the order of signals has sufficient resolution from the other signals for the other nucleobases.

In Table 1 above, the relative time for a single pass through a construct is reported relative to the “raw code”. Herein the terminology “a single pass” is a measure of the relative time it takes to sequence the polymer 110 from start to end. The time is measured in relation to a polymer 110 encoding the “raw code” (F-1-4) as a reference. As the actual length in time (e.g. in milliseconds) will depend on the particular biophysical characteristics of the reporter moieties, the ARCs, spacers and linkers used. For example, for a sample polynucleotide (e.g. of fixed length and nucleotide composition) encoded as a polymer 110, a value of 2 for the “time for a single pass” signifies it will take about two times as long to sequence a polymer 110 created with F-2-5 EC nucleotides (shown in FIG. 5) than an equivalent polymer 110 created with F-1-4 nucleotides (shown in FIG. 3).

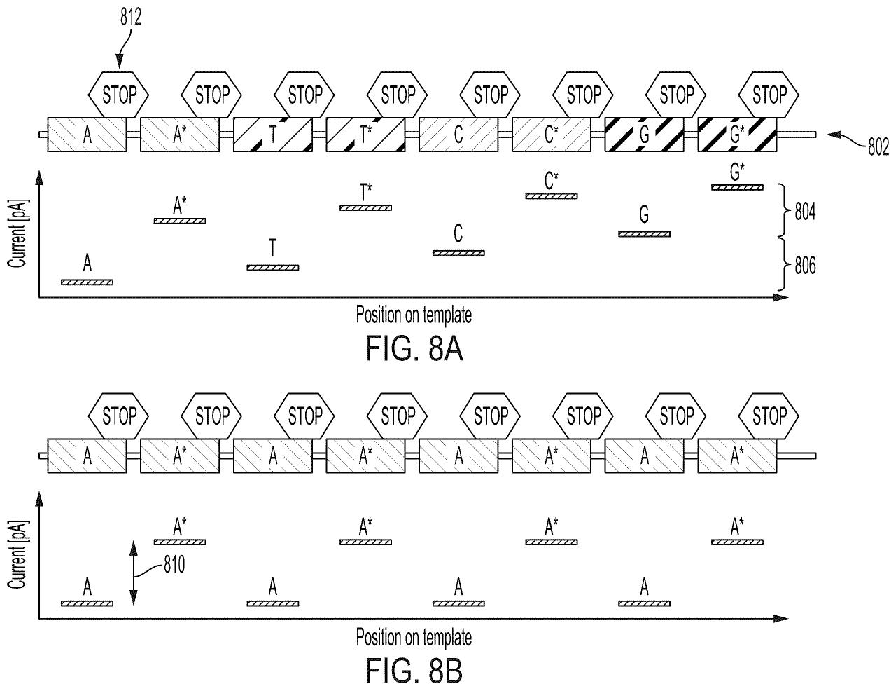

Additionally, Table 1 above shows the median and minimum signal distances as a fraction of the total current range. Herein, the “median signal distance” and “minimum signal distance” are measurements of the expected separation of reporter signals. For these calculations, it was assumed that all reporter signals are evenly distributed across a pre-determined range; additionally, the values for all signals were normalized to the unit interval (e.g. from 0 to 1) by dividing all signal values (e.g. currents) to the value of the highest signal. For example, for the “raw code” (F-1-4), which has 4 unique reporter currents (e.g. one for each base), the four normalized signal values are 0.25, 0.5, 0.75, 1. The minimum signal distance and median signal distances is computed by enumerating all pairwise combinations of EC nucleotides, and listing signal separations (e.g. absolute difference between the current values) between all adjacent EC reporter values. From the resulting list of signal separations, the minimum value and the median value is extracted. If the median value for a list is between two numbers, their average is reported. This is discussed further with respect to FIGS. 8A and 8B below.

In the implementation of EC nucleotides, several factors should be considered. First, in some examples, an EC nucleotide should maintain a minimum deletion distance between reporters in the encoding. The deletion distance is the number of signals that can be deleted in a reporter before the identity of the encoded base is lost. A deletion occurs when the pulse duration is longer than the time taken to translocate the distance from the first arresting construct to the second arresting construct. In this case, the pulse is still “high” when the second arresting construct arrives. So the minimum deletion distance is a function of D=RT (Distance=Rate*Time). The deletion distance can be both a structural feature (e.g. generated by an appropriate choice of spacer segment length and effective charge between EC reporters) and functional feature in the design choice of the EC nucleotide (e.g. in choosing an appropriate ordering of EC reporter signals such that a single, double, or multiple deletion has a desired outcome for the identification of the EC nucleotide based on the order of measured EC reporter signals). This is generally the basis of deletion error tolerance.

Second, in some examples, an EC reporter should maintain a minimum insertion distance between reporters in the encoding. The insertion distance is the number of signal values that can be added to a reporter before the identity of the encoded base is lost. The insertion distance can be both a structural feature (e.g. generated by an appropriate choice of EC reporter signal to noise ratio, or appropriate choice of arresting construct “strength” with respect to stimulus) and functional feature in the design choice of the EC nucleotide (e.g. in choosing an appropriate ordering of EC reporter signals such that a single, double, or multiple deletion has a desired outcome for the identification of the EC nucleotide based on the order of measured EC reporter signals). This is generally the basis of insertion error tolerance.

Third, in some examples, an EC nucleotide should maintain a minimum Hamming distance between reporters in the encoding. The Hamming distance is the number of reporter signal values that can be changed before the identity of the encoded base is lost. This is generally the basis of mismatch error tolerance. This notion of the Hamming distance (as well as deletion and insertion distances above) should be understood to be design guidelines/principles for the reporter codes that are selected (e.g. on how to choose suitable F-X-X, or B-X-X codes). The minimum deletion distance, the minimum insertion distance, and the minimum Hamming distance are not intended to be “physical” guidelines on how to build the molecule, but in some examples they can be used as such. Note that there are discrete states being mapped (e.g., discrete reporter states that can map to base identities either uniquely or in combination). While the signal which is read is analog, the “state” corresponding to the base or reporter identity is discrete. As such, the Hamming distance as well as “insertion/deletion channels” (where the insertion distance and deletion distance terminology come from) may be considered to be relevant to, though not controlling of, molecular design.

Fourth, in some examples, an EC nucleotide should seek to increase or maximize the number of “higher current” to “lower current” transitions between each EC reporter, and across the set of all reporters (including between adjacent EC nucleotides). This lowers the requirements for the minimum signal to noise ratio (SNR).

Fifth, in some examples, the EC nucleotide should seek to reduce or minimize the length of an encoding. This improves overall sequencing rates by requiring fewer ARCs (i.e., EC reporter current readings) per nucleotide, as each ARC requires a minimum amount of sequencing time. Finally, an EC nucleotide should seek to eliminate indistinguishable transitions between adjacent EC reporter signals (e.g. current reads). This removes needing foreknowledge of secondary metrics such as the mean duration of a current level to resolve transitions between ARCs.

With regard to eliminating indistinguishable transitions between adjacent EC reporter signals (e.g. current reads), there are general constraints in nanopore sequencing that are not widely experienced in other binary communication protocols, such as telecommunications. For example, in a binary protocol, a message such as “0001110101” can be transmitted, received and decoded with relatively high accuracy. However, in ratchet-based nanopore sequencing such a binary message may not be reliably received. One reason why a binary message such as this may be interpreted incorrectly in ratchet-based nanopore sequencing is because the sequencing by “forward ratcheting” (e.g., using a constant current or positive pulses) generally transition from one ARC position to the next stochastically. In other words, the advancement of the translocation may be modeled according to general distributions (such as bell curves, or exponential curves) in statistics, but the advancement of the sequence through the nanopore cannot be precisely predicted. Therefore, the binary message “0001110101” could be read as “010101” when translocated through a nanopore. As such, binary codings, such as having only two distinct current signals (0 or 1) may be less effective at reading polynucleotide sequences. Therefore, the EC reporters in Table 1 use at least three distinct current signals for reading the various nucleobases.

Advantageously, an EC nucleotide may be optimized to provided information indicating a sequencing readout error has occurred, and the type of error (e.g., single deletion, double deletion, insertion, etc.) for each reporter. This type of information from the errors can help facilitate quality controls in post-processing as well as possible quality controls that can occur during the sequencing operation itself, for example, by adjusting the instrument and correcting readout errors in real time. Non-limiting examples of real-time error adjustments include altering instrument parameters to reduce or minimize errors as sequencing proceeds through a construct in order to inhibit or prevent future read cycles from experiencing the same errors or, in the case of bipolar EC reporters, this may involve detecting that a readout error has occurred and “back-tracking” through the construct to attempt to fix the observed error locally and obtain a correct reading.

### F-2-8 EC Nucleotide Example

FIGS. 8A and 8B depict an example readout associated with an F-2-8 EC nucleotide. FIG. 8A depicts the example sequence of monomer units (e.g., sequence of cyclic loops 802) for an example sample polynucleotide sequence with the order of A-T-C-G. In this example, each monomer unit corresponding to a respective nucleotide has a cyclic loop with two EC reporters and two arresting constructs, one arresting construct adjacent to (e.g., after) each EC reporter. In some examples, the monomer unit (e.g., cyclic loop) may also include a spacer element to increase the distance between successive reporter elements in the EC construct of that monomer unit. The sequence shown in FIGS. 8A and 8B is unique from the sequence shown in FIG. 7, for example, in that there is no generic “tick mark” for all of the nucleobases. The sequence in FIGS. 8A and 8B has a unique EC reporter (A*, T*, C*, G*, which may be thought of as being similar to a tick mark) in addition to a nucleobase reporting region (A, T, C, G). Thus, the readout in FIGS. 8A and 8B (for a polymer encoded with F-2-8 EC nucleotides) is produced with eight unique current signals. As shown in FIG. 8A, each EC nucleotide includes a first EC reporter (illustratively, A) and a second EC reporter (illustratively, A*). In this example, the second EC reporter is associated with a higher current signal 804, and the first EC reporter is associated with a lower current signal 806. In this manner the signals may be deciphered with higher fidelity. For example, the signal 806 from the first EC reporter may be used as a first confirmation of the corresponding nucleotide's identity, and the signal 804 from the second EC reporter may be used both to confirm that the reporter has translocated out of the nanopore's aperture and as a second confirmation of the corresponding nucleotide's identity. As illustrated in FIG. 8B, the signals 804 from the EC reporters may serve a similar function as tick marks described with reference to FIG. 6B when a homopolymer region is being sequenced.

It will be appreciated that any suitable combination of signal levels may be used. However, in a manner such as illustrated in FIG. 8B, having a system with eight unique signals in the various EC reporter regions of each nucleobase should advantageously provide a large difference 810 in current between reporter signals (AI) in order to clearly differentiate between distinct signals. In some examples, the alternating higher and lower signals (e.g., signals 804 and 806) corresponding to each nucleobase allows for a lowering of the signal to noise ratio and improves the fidelity of the readout. However, eight unique signals may be more difficult to implement, as the AI 810 between the various signals decreases as the number of unique signals increases. For example, in FIG. 8A the lowest of the higher current EC reporter signals 804 (A* for example) is brought closer to the highest of the lower current reporter signals 806 (G for example) as the number of unique signals increases. Additionally, the distance 810 between each of the respective higher current EC reporter signals 804 (A*, T*, C*, G*) and lower current reporter signals 806 (A, T, C, G) is brought closer together with each unique signal that is added to the various cyclic loops. In some circumstances, this can reduce resolution.

FIG. 5 shows an example general structure for an F-2-8 EC nucleotide. As discussed above, the tick mark 510 in FIG. 5 (as previously discussed herein in the context of an F-2-5 “tick-mark” nucleotide) is not limited to a single distinct signal in an F-2-8 EC reporter. In an example F-2-8 EC nucleotide, there can be one of four distinct tick mark-like EC reporters 510 that generate signals that are associated with each of the four bases (such as A*, T*, C*, G*), in addition to one of the four distinct reporters that generate current signals for the nucleobases themselves (A, T, C, G). As discussed with regard to FIG. 8A, each of A*, T*, C*, G* EC reporters 510 may be configured to generate higher current signals 804 and each of A, T, C, G reporters 508 may be configured to generate lower current signals in order to increase the difference AI between the various current signals and increase the signal to noise ratio. In other examples, EC reporters 508 instead may generate higher current signals and EC reporters 510 instead may generate lower current signals.

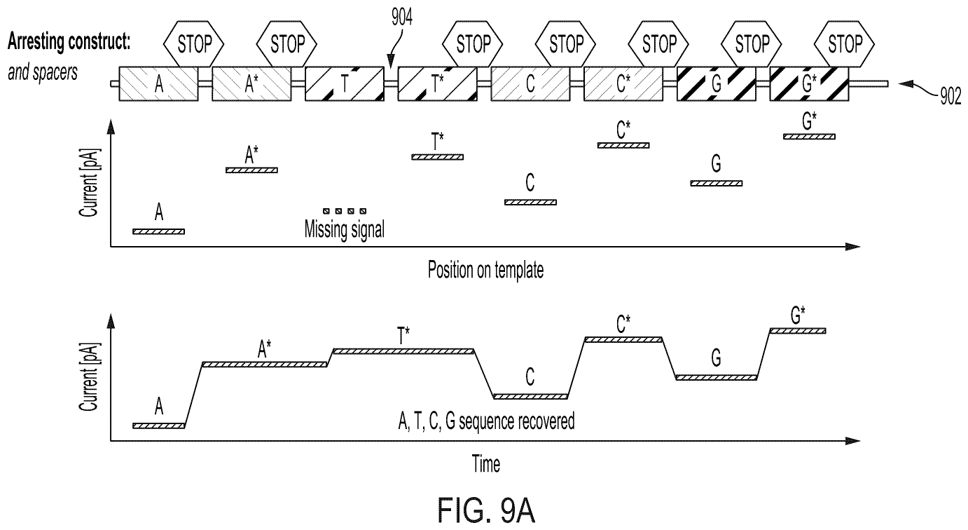

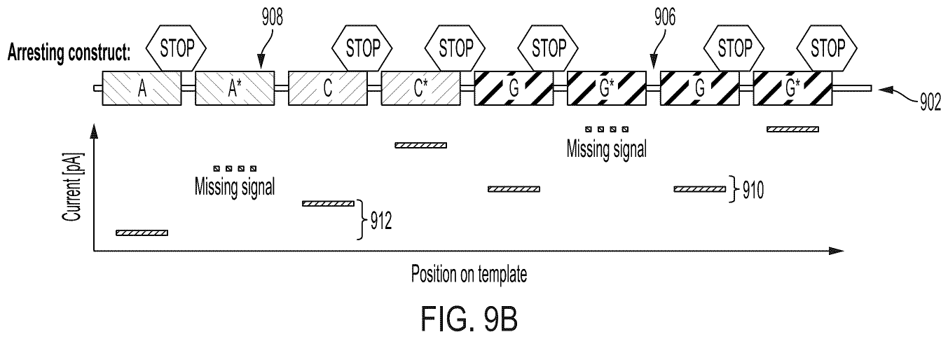

As shown in FIG. 9A, an F-2-8 nucleotide is resilient to many forms of single deletion errors. For example, FIG. 9A contains a cyclic loop sequence 902 encoding A-T-C-G using monomer units which include a first EC reporter region, an arresting construct adjacent to the reporter region, a second EC reporter, and an arresting construct adjacent to the second EC reporter. However, even in a nonlimiting example with a damaged or skipped arresting construct 904 between the A* and T* reporters, an error in the readout is identifiable and may be eliminated. For example, the absence of a signal corresponding to T may indicate that an arresting construct 904 was missed. Additionally, the signal corresponding to T* is distinguishable from the signal corresponding to A*, so the T in the sequence can still be identified based on the signal from T's EC reporter even if the reporter for T is not itself read. Thus, having four unique tick-mark-like reporters in the F-2-8 sequence may reduce or eliminate the likelihood of a readout error, such as the readout error in FIG. 7. As such, in the nonlimiting example illustrated in FIG. 9A, the correct sequence of A-T-C-G may be identified despite the deletion error (missing read of T reporter).

One drawback of the F-2-8 reporter relates to readout errors in homopolymer sequences where the higher current signal (in this case G*) has a damaged or skipped arresting construct 906, such as that shown in FIG. 9B. Thus, the transition between the monomer units of the homopolymer G-G was not recognized and an incorrect readout of A-C-G was recorded, instead of the correct sequence of A-C-G-G. This is because the transition in signal 910 between the two “G” reporters was not recorded. However, the F-2-8 reporter was able to recognize a damaged or skipped arresting construct 908 in the heteronucleotide sequence A-C, resulting in the recovery of a transition signal 912.

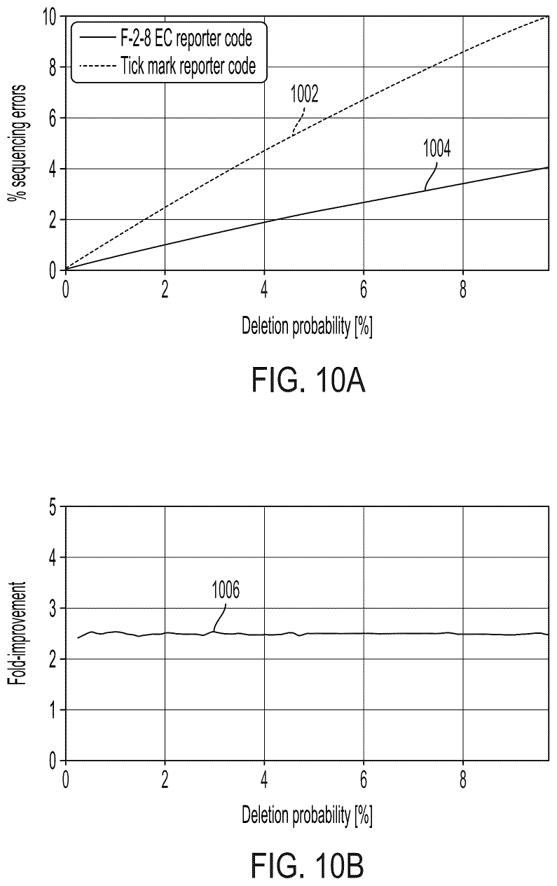

FIG. 10A depicts a computational comparison of the percentage of sequencing errors for a random sample polynucleotide sequence encoded with F-2-8 EC nucleotides (solid line 1004) to that of an F-2-5 EC nucleotide including a generic tick mark reporter (dashed line 1002). FIG. 10A depicts the deletion probability on the x axis and the percent of sequencing errors on the y-axis. As shown in FIG. 10A, an F-2-8 EC reporter advantageously provides exceptional reduction in deletion probability and an overall reduction in sequencing errors compared to an F-2-5 tick mark reporter. FIG. 10B shows a computational comparison of the improvement of an F-2-8 reporter (solid line 1006) as compared to an F-2-5 tick mark reporter. The deletion probability is graphed on the x-axis with the fold of improvement (as compared to a single tick mark reporter in an F-2-5 scheme) on the y-axis. Advantageously, the EC F-2-8 nucleotide provides more than a two-fold improvement (e.g., approximately a 2.5-fold improvement) that does not materially change with respect to deletion rate.

### F-3-4 EC Nucleotide Examples

An F-3-4 EC reporter is advantageous in that it lowers the requirements for signal to noise ratio. As discussed above with respect to the EC F-2-8 nucleotide, using 8 distinct signals may cause the signals to decrease in AI between the various signals. However, with an F-3-4 EC reporter, the AI increases, as there are fewer unique signals to be read. This type of EC nucleotide can be advantageously applied to applications with inherently low SNR (such as optical detection of nanopore currents), although this type of EC nucleotide suitably may be implemented in applications with higher SNR as well.

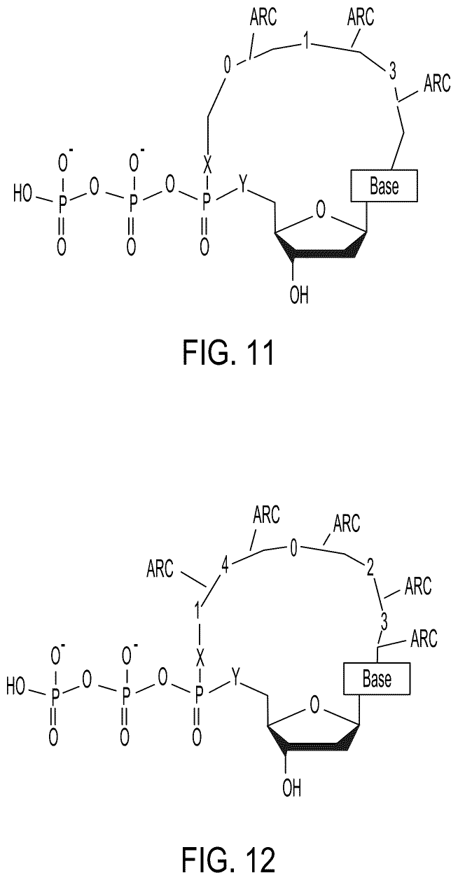

FIG. 11 depicts an example general scheme for an F-3-4 EC reporter modified nucleobase. In this example, the cyclic loop comprises three distinct EC reporter signals (“0” “1” and “3”) as well as three arresting constructs. Although not shown, a fourth distinct signal (i.e., “2”) may be substituted for the EC reporters to code for different bases (for example A: [0, 1, 3]; T: [0, 2, 1]; C: [2, 0, 3]; G: [2, 3, 1]). Also not shown may be various linkers or spacers that can advantageously provide distance between successive reporter elements.

In some examples, an F-3-4 EC code can be characterized by any suitable combination of the following features. First, the reporters within the code can be combined in arbitrary orders to encode a nucleotide sequence and are built into the cyclic loop construct for each nucleotide (for example, A [0, 1, 3] vs. T: [0, 2, 1]). Second, the code has three distinct current signatures per nucleotide used to encode the bases of a DNA sequence, allowing to clearly resolve homopolymer regions and other sequences. Third, the encoding has a partial tolerance to deletion, insertion, and mismatch errors, and single errors (if such errors occur) are readily identified with a look-up table. Fourth, the code has a signal that substantially differs from one reporter to the next due to the relatively low SNR requirements (only four reporter currents need to be identified). This final feature is advantageous as it helps lower the probability of mismatch and insertion errors and can result in overall faster sequencing rates due to the lower requirements for the signal integration time (i.e., read time).

One potential limitation of an F-3-4 EC reporter is that it may require more passes of the molecule through the nanopore in order to achieve a total error rate of < 1/1000 (i.e., longer net sequencing time for a total accuracy of Q30 relative to F-2-8). However, the higher SNR of the F-3-4 EC reporter example could enable a shortened integration time. Therefore, as the F-3-4

EC reporter example may require twice as many passes through the molecule (12 passes for F-3-4 vs. 6 passes for F-2-8), the sequencing time may not necessarily be doubled over all.

### F-5-5 EC Nucleotide Examples

FIG. 12 depicts a general scheme for an F-5-5 EC reporter modified nucleobase. The cyclic loop comprises five distinct EC reporters for generating respective signals as well as five arresting constructs. Although not shown, various linkers or spacers that can advantageously provide distance between successive reporter elements. In some examples, each EC reporter element (i.e., 0, 1, 2, 3, 4, or 5) may be spaced from another reporting element by a spacer in order to provide distance and increase resolution. If correlated with the examples in Table 1, the nucleobase encoded in FIG. 12 (1, 4, 0, 2, 3) would be coded for Adenine (A). However, the encoded bases in Table 1 are purely illustrative, and any combination of sequences may be used to encode certain nucleobases.

In some examples, an F-5-5 EC code can be characterized by any suitable combination of the following features. First, the EC reporting elements can be combined in arbitrary orders to encode a nucleotide sequence and are built into the cyclic loop construct for each nucleotide. Second, the EC reporting elements have five different current signatures to encode respective nucleotides, and it allows to clearly resolve homopolymer regions and other sequences. Third, the encoding has a full tolerance to single deletion, insertion and mismatch errors, and partial tolerance to double deletion, mismatch, and insertion errors. If such errors occur, the errors may be readily identified with a look-up table. Fourth, the EC reporting elements have a signal that appreciably differs from one template position to the next.

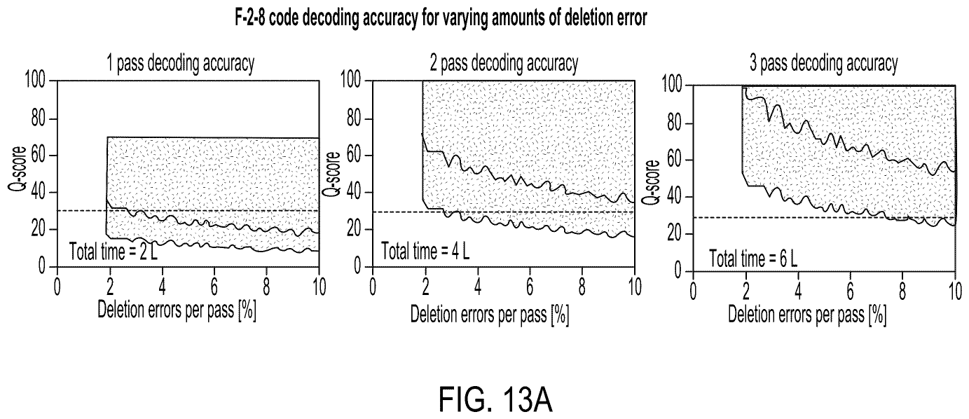

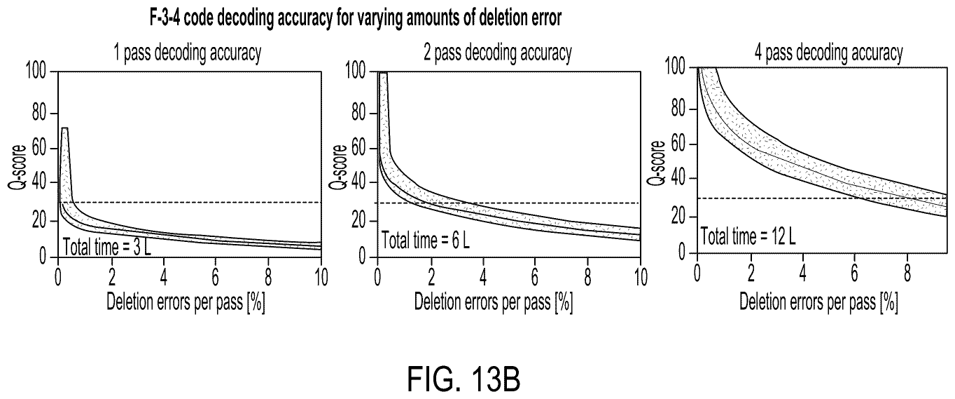

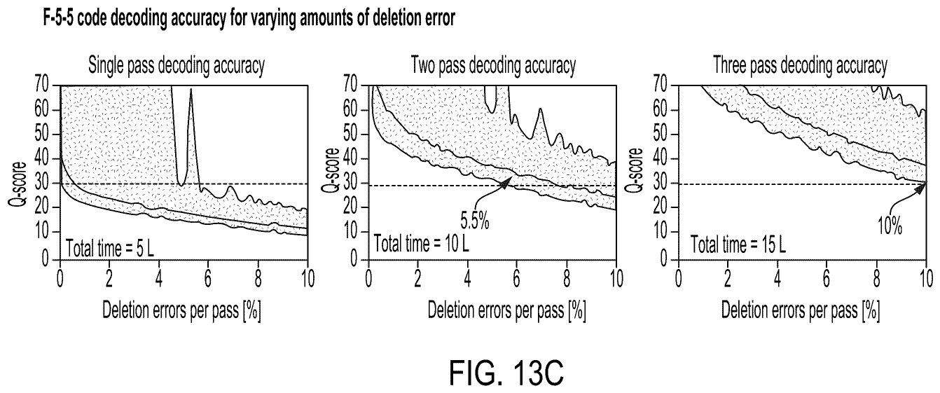

As shown in FIG. 12, the EC reporting elements for an F-5-5 sequence have additional arresting constructs (ARC) compared to an F-3-5 or an F-2-5 configuration. An increase in the number of reporting elements and arresting constructs generally results in an increase in the chemical synthesis complexity of each of the constructs for A, T, C, G, and henceforth the total cost of producing daughter strands for nanopore sequencing. However, despite the longer construct length, the F-5-5 code achieves Q30 with a similar rate as the tick-mark code (i.e., requiring 10 L total sequencing time). This is discussed further with respect to FIG. 13C.

Finally, one particular advantage the F-5-5 scheme has is that it provides a full error log in the case of mismatch errors, insertion errors, and deletion errors. Because of the redundancy and the number of signals in each cyclic loop, each error may be identified and mapped accordingly. This may provide particular insights into the sequencing process as well as allows for relatively high accuracy in the sequencing readout.

### Q30 for F-2-8, F-3-4, and F-5-5

FIGS. 13A, 13B, and 13C depict a computational comparison of the F-2-8, F-3-4, and F-5-5 schemes with deletion errors per pass (as a percentage) versus the Q-score for each time period. The Q score is measured as Q=−10 log 10 (error probability). The Q30 benchmark accuracy is shown in each figure subpanel by a dashed line. To generate the Q-score lines shown in the plots, one thousand random sample polynucleotide sequences of length 100 bases were generated, and each polynucleotide sequence was converted to a series (list) of signals corresponding to an EC reporter code using the encoding schemes shown in Table 1. Each reporter signal in the list was then either kept or randomly discarded using a uniform random number generator. For example, for a 5% deletion error rate, a random number between 0 and 1 was generated and if the value is below 0.05 the reporter signal was deleted from the list resulting in a “corrupted” list of signals. Then, using the “corrupted” list of signals, the signals were “decoded” back into a predicted polynucleotide sequence using a lookup table. Using pairwise alignment between the predicted polynucleotide sequence (i.e., the sequence decoded from the corrupted signal) and the ground truth sequence (i.e., the original, uncorrupted polynucleotide sequence), a basecalling accuracy was computed and stored. The mean basecalling accuracy and its standard deviation was then converted to a Q-score and plotted in FIGS. 13A to 13C for each sampled percentage of deletion error from 0% to 10%. To compute the expected Q-scores associated with two and three passes (flosses), the percentage error from the single-passes were squared or cubed, respectively and the resulting residual errors were then converted to Q-scores; this procedure assumes that the errors between each floss (pass) are uncorrelated.

The net sequencing time (e.g. indicated in each figure subpanel as “total time”) is reported relative to the sample polynucleotide (e.g. DNA) sequence length L in numbers of encoded bases (e.g. monomer units) multiplied by the number of times the molecule is flossed (i.e., indicated by the number of “passes”). This accounts for the time spent reading all the reporters as well as reading through the polymer more than one time. For example, as an illustration, a sample polynucleotide of length L=100 bases may create a polymer (e.g. similar to polymer 110) with 200 reporters for an F-2-8 code (FIG. 13A), 300 reporters for an F-3-4 code (FIG. 13B), or 500 reporters for an F-5-5 code (FIG. 13C). Therefore, the “total time” for a single pass will be 2 L (i.e., 200 units of time), 3 L (300 units of time) and 5 L (500 units of time) respectively. The relative time can also be thought of as the sequencing time relative to an F-1-4 encoded polymer. For the cases where multiple flosses (e.g. “passes” or “re-sequencing”) of the polymer is performed, the total time is reported as the “single pass” time, multiplied by the number of flosses. In the above example, the “total time” for two flosses (two passes) will be 4 L (i.e., 400 units of time) for the F-2-8 code, 6 L (600 units of time) for the F-3-4 code, and 10 L (1000 units of time) for the F-5-5 code. FIG. 13A sequentially depicts the F-2-8 scheme with 1 pass decoding accuracy (total time of 2 L), 2 pass decoding accuracy (total time of 4 L), and 3 pass decoding accuracy (total time of 6 L). FIG. 13B sequentially depicts the F-3-4 scheme with 1 pass decoding accuracy (total time of 3 L), 2 pass decoding accuracy (total time of 6 L), and 4 pass decoding accuracy (total time of 12 L). FIG. 13C sequentially depicts the F-5-5 scheme with 1 pass decoding accuracy (total time of 5 L), 2 pass decoding accuracy (total time of 10 L), and 3 pass decoding accuracy (total time of 15 L).

As stated with respect the F-5-5 schemes, the projected time for reading the F-5-5 cyclic loop EC reporters may be longer for a single pass (5 L for F-5-5 versus 3 L or 2 L) as the F-5-5 scheme incorporates 5 different unique signals on each cyclic loop, increasing the number of arresting constructs and the total time for the readout. However, after only two passes (total time 10 L) the F-5-5 scheme projection exceeds the Q-30 threshold (at a 5% deletion rate for over 68% of the molecules), whereas the F-3-4 achieves the Q30 threshold (at a 5% deletion rate for over 68% of the molecules) after 4 passes (total time 12 L) and the F-2-8 scheme exceeds the Q30 (at a 5% deletion rate for over 68% of the molecules) threshold after 3 passes (total time 6 L). Therefore, the F-5-5 scheme requires a fewer number of passes to exceed the Q30 threshold due to having 5 unique signals on each cyclic loop, providing the redundancy for relatively high fidelity readouts.

FIG. 13A shows that F-2-8 is the fastest overall sequencing scheme, with the lowest total time to exceed the error threshold for Q30. However, as discussed in greater detail below each of the EC reporter schemes may provide a unique benefit in each scenario, and fastest overall sequencing time may have tradeoffs for lower error reporting, etc.

B-6-4 EC nucleotide Examples

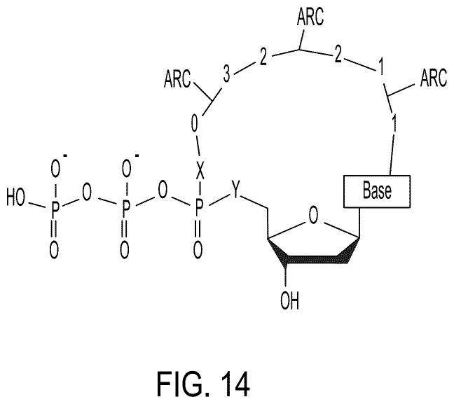

FIG. 14 depicts a general scheme for a B-6-4 EC reporter modified nucleobase. The cyclic loop comprises four unique EC reporter signals (0, 1, 2, 3) with six sequential signals (e.g., [0, 3, 2, 2, 1, 1]) and three arresting constructs. The first of the six sequential signals (0) is provided before an arresting construct, the next two signals (3 and 2) are provided before the next arresting construct, the next two signals (2 and 1) are provided before an arresting construct and the last signal (1) is provided adjacent to the base. Although the depiction of these reporter has been listed sequentially, there is no single readout direction. Since the B-6-4 code operates as a bipolar code, it may be read forwards ([0, 3, 2, 2, 1, 1]) or backwards ([1, 1, 2, 2, 3, 0]). Further, a person having ordinary skill in the art could foresee that a coding scheme could operate with a palindromic sequence or ring structure (i.e. 3, 2, 1, 0, 1, 2, 3) and be read forwards or backwards. With the modified nucleobase of FIG. 14, the arresting constructs provide a type of palindromic order, with one signal flanking the ends of the arresting constructs and two signals nestled between the arresting constructs (creating a one, two, two, one encoding scheme). The arresting construct flanked by reporters allows the positioning of the correct reporter in the readhead depending on the read voltage polarity. The arresting construct should be capable of pausing (stalling) the reporter in the nanopore sequencing readhead when the ARC is in the pore vestibule (under positive biases) and when the ARC is beneath the nanopore sequencing readhead (under negative biases)

Although not shown, various linkers or spacers that can advantageously provide distance between successive reporter elements. In some examples, each EC reporter element (i.e., 0, 1, 2, or 3) may be spaced from another reporting element by a spacer in order to provide distance and increase resolution. If correlated with the examples in Table 1, the modified nucleobase in FIG. 14 would be coded for Adenine (A) in the forward and reverse direction. However, the encoded bases in Table 1 are purely illustrative and various combinations of sequences may be used to encode certain nucleobases.

With the forward ratcheting mechanisms discussed previously (such as F-#-#), a single reading step may be performed between each ratcheting event. However, with a bipolar EC coding scheme, two reading steps may be performed between each ratcheting event, under a positive and negative voltage (or a forward and reverse translocation movement). Thus, in some examples the B-6-4 is bipolar as the read may be performed both in the forward direction at a first time and in the reverse direction at a second, different time. Further, bipolar reading can be performed (occur) between two arresting constructs (in forward and reverse directions), between two nucleobases (forward and reverse), between a predefined set of nucleobases (forward and reverse) or along the entire length of a polymer encoding a DNA or RNA strand, both in forward and reverse.

In some examples, a B-6-4 EC reporter scheme can be characterized by any suitable combination of the following features. First, the signals can be combined in arbitrary orders to encode a nucleotide sequence and are built into the cyclic loop construct for each nucleotide. For instance, the order of the A, T, C, and G nucleobases should not affect the resolution of the readout, whether in a forward translocation event or in a reverse translocation event. Second, the scheme has four distinct current signatures used to encode respective nucleotides and allows to clearly resolve homopolymer regions and other sequences. Third, the encoding has a tolerance to single deletion, insertion and mismatch errors. If such errors occur, such errors are also readily identified with a look-up table. Fourth, the code has a signal that substantially differs from one template position to the next. Fifth, the code is operatable in a “bipolar read mode”, which is compatible with ratcheting with alternating current waveforms. The alternating current waveform allows to improve nanopore device longevity by reducing or minimizing the ionic depletion over time. As discussed previously, the sequence of signals also should be resolvable in a forward and reverse bias voltage.

The encoding of the cyclic loop in a B-6-4 scheme is advantageous in that it is compatible with AC waveforms (oscillating forward and reverse currents) while still providing full deletion error tolerance, full insertion error tolerance, and full mismatch error tolerance.

The B-6-4 scheme provides other desirable advantages to a sequencing operation. For example, the bipolar reading mechanism can extend the lifetime of the sequencing nanopore. This is partially because the trans well electrolyte is recharged in real-time when a negative voltage is applied to the nanopore. Secondly, the bipolar EC codes may reduce the chemical complexity of synthesizing the daughter strand due to their symmetry. For example, two reporters for generating reporter signals can be provided between each arresting construct, this reduces the numbers of arresting constructs required in each cyclic loop while still providing additional information between each arresting construct. Further, there are four unique signals, which reduces the numbers of signals that are synthesized in the daughter strand (compared to other schemes that have 5 or 8 unique signals). Finally, the capability to read reporters under a negative bias provides additional information simply by its position with respect to an arresting construct (for example if two reporters are before an arresting construct or if only one reporter is adjacent to an arresting construct). Information therefore can be encoded by manipulation of the reporter's position (below or above the ARC), rather than by its chemical structure and associated current blockade. This advantageously reduces the structural diversity that is required of the construct (for example only requiring 4 unique signals and not 5 or 8). However, a person having ordinary skill in the art could foresee a bipolar scheme with more than 4 unique signals to encode additional information or provide additional redundancy and provide a more complete error log. It is also possible to provide a bipolar current scheme with fewer than 4 unique signals, such as the B-6-3 scheme shown in Table 1.

In some examples, the implementation of a bipolar scheme may be chosen for the appropriate nanopore sequencing event. For example, negative bias ratcheting events may be more error prone (such as to auto-ratcheting or skips) than forward bias ratcheting events. Therefore, the nanopore for a bipolar scheme should ideally have relatively high predictability in both the forward ratcheting and the reverse ratcheting operations. Further, it may be desirable to provide predictable chemical structures for the EC reporters (0, 1, 2, 3, etc.) in a bipolar scheme in order to ensure that the directionality of the incorporated cyclic loop nucleotide is consistent in order to preserve the robustness of the EC-code. Further, providing more straightforward and predictable chemical structures in the cyclic loop can lower the complexity in the synthesis of the cyclic loop. Finally, if uncontrolled, either the forward or reverse code could be read as the strand is ratcheted through the nanopore in bipolar schemes. This could be problematic for an approach relying on fewer unique reporters (e.g., B-6-3). Therefore, it may be desirable to provide increased redundancy to the sequence in order to provide a reduced error (and in some examples, error-proof) mechanism to troubleshoot the direction of the readout. For example, it may be desirable to provide a bipolar code with at least four unique signals (such as B-6-4) as opposed to a bipolar scheme with only three unique signals (B-6-3). In this manner it may be easier to identify a mixture of signals that may be unpredictably advanced in both a forward and reverse direction. As discussed before, a person of ordinary skill in the art could foresee a variety of signal sequence orders in order to detect and troubleshoot potential issues such as this. For example, a bipolar encoding scheme could have a palindromic-type sequence or an algebraic ring structure that could provide redundancy in both a forward and reverse direction.

### Comparison of Select Schemes

In total, the various schemes have advantages and disadvantages, some nonlimiting examples of which have been discussed herein. For example, raw code (F-1-4) is synthetically advantageous as the monomer units (e.g., cyclic loops) are only provided with a single unique reporter to generate a corresponding unique signal from which a nucleotide may be identified. This reduces the complexity of the coding operation. However, raw code is not fully or partially error-proof to single or double deletion error, single or double deletion insertion error, or single or double deletion mismatch tolerance. The time to process raw code to Q30 (at a 5% deletion error rate for >68% of molecules) is >30 L, and the raw code does not provide an error log.

The tick mark scheme (F-2-5) is advantageous in part because it is partially tolerant to single deletion errors, insertion errors, or mismatch errors but is not tolerant to double deletion errors, insertion error, or mismatch errors. The time to process to Q30 (at a 5% deletion error rate for >68% of molecules) is 10 L and it provides a partial error log. The tick mark scheme may be particularly advantageous for certain applications in that the output of the processed signals is human readable, such as after converting the current, voltage, or optical signals to numbers or letters.

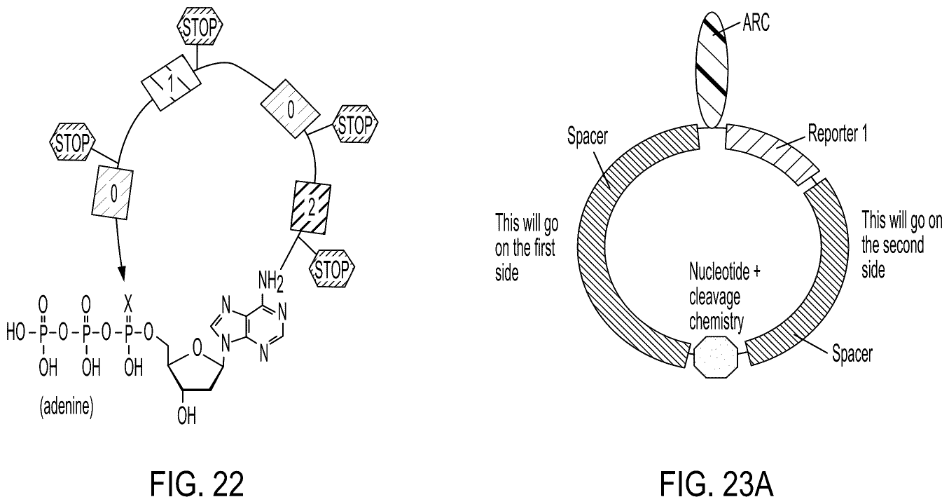

The F-2-8 EC coding scheme is advantageous in part because it is partially tolerant to single and double deletion errors, single and double insertion errors, and single and double mismatch errors. The time to process the sequence to reach Q30 (at a 5% deletion error rate for >68% of molecules) is 6 L and it provides a partial error log. In addition, the F-2-8 EC coding scheme may be particularly advantageous for certain applications in that it is the fastest overall sequencing operation when compared to raw code, tick marks, or the F-3-4, F-5-5, F-4-3 or bipolar schemes. An example F-4-3 scheme is illustrated in FIG. 22, in which it may be seen that there are four arresting constructs and four reporter moieties (0, 1, 0, 2) chosen from only three different types of reporter moieties (0, 1, 2, and 3). Although adenine is the nucleobase in this example, other nucleobases suitably may be used.

The F-3-4 EC coding scheme is advantageous in part because is partially tolerant to single deletion errors, single and double insertion errors, and single and double mismatch errors. The time to reach Q30 (at a 5% deletion error rate for >68% of molecules) is 12 L and it provides a partial error log. The F-3-4 EC scheme may be particularly advantageous for certain applications in that it has lower signal to noise ratio requirements (as it generally uses 3 unique signals with larger separation between the signal).

The F-5-5 EC coding scheme is advantageous in part because it is fully tolerant to single deletion errors, single insertion errors, and single mismatch errors. Furthermore, it is partially tolerant to double deletion errors, double insertion errors, and double mismatch errors. The time to reach Q30 (at a 5% deletion error rate for >68% of molecules) is 10 L and it provides a substantially full error log. The F-5-5 EC scheme may be particularly advantageous for certain applications because it provides the fullest error log and has a relatively low amount of time to reach Q30, which is unusual for the number of distinct signals encoded in each cyclic loop.

Finally, the B-6-4 EC scheme is advantageous in part because it is fully tolerant to single deletion errors, single insertion errors, and single mismatch errors. It is partially tolerant to double insertion errors and double mismatch errors as well. It does provide a partial error log and may be particularly useful in certain examples or with certain nanopore structures in that it provides for alternative current reads.

Table 2 below summarizes example, selected features of certain schemes described herein.

TABLE 2

| | | # | Deletion error | | Insertion error | | Mismatch error | | Time | | |
|---|---|---|---|---|---|---|---|---|---|---|---|
| | # of | unique | tolerance | | tolerance | | tolerance | | to | Error | Example |
| Name | ARCs | barcodes | Single | Double | Single | Double | Single | Double | Q30 | log | Feature |
| Raw | 1 | 4 | N | N | N | N | N | N | >30 L | N | — |
| code | | | | | | | | | | | |
| Tick | 2 | 5 | + | N | + | N | + | N | 10 L | + | Human |
| marks | | | | | | | | | | | readable |
| F-2-8 | 2 | 8 | + | + | + | + | + | + | 6 L | + | Fastest |
| | | | | | | | | | | | overall |
| | | | | | | | | | | | sequencing |
| F-3-4 | 3 | 4 | + | N | + | + | + | + | 12 L | + | Lower |
| | | | | | | | | | | | SNR |
| | | | | | | | | | | | requirements |
| F-5-5 | 5 | 5 | ++ | + | ++ | + | ++ | + | 10 L | ++ | Best error |
| | | | | | | | | | | | reporting |
| B-6-4 | 3 | 4 | ++ | N | ++ | + | ++ | + | — | + | Alternating |
| | | | | | | | | | | | current reads |

Symbols meanings: ++ (Full), + (partial), N (None).
The time to Q30 is computed for a 68% yield above Q30 and a 5% deletion error rate. The time to Q30 is reported relative to the total DNA sequence length L in numbers of encoded bases. This accounts for the time spent reading all the reporters as well as reading through the construct several times. Naming convention for constructs is F- for “forward only” reads, B- for bipolar reads (i.e. “forward and reverse” reads for use with AC waveforms). The two numbers that follow indicate the code word length and the number of unique reporter currents required. Following this convention, the “tick-mark” code is an F-2-5 code and the “raw code” is an F-1-4 code. Example molecular designs for these constructs are described elsewhere herein.

### Candidate Reporter Structures

To provide distinct signals during passage through the nanopore, various candidate chemical structures have been developed. Some linking structures may operate as spacers and reporting regions. For example, one or more synthetic polymer sequences may be used to space out the reporting regions as well as provide a unique signal for the nucleobase. It may be envisioned by a person having ordinary skill in the art could synthesize two, three, or more consecutive sequences of the polymers below. However, doing so may slow down the nanopore sequencing operation. Thus, a person having skill in the art would evaluate the tradeoff between additional repeating sequences, synthetic complexity, and time required to sequence the nanopore to a suitable accuracy (such as a Q30).

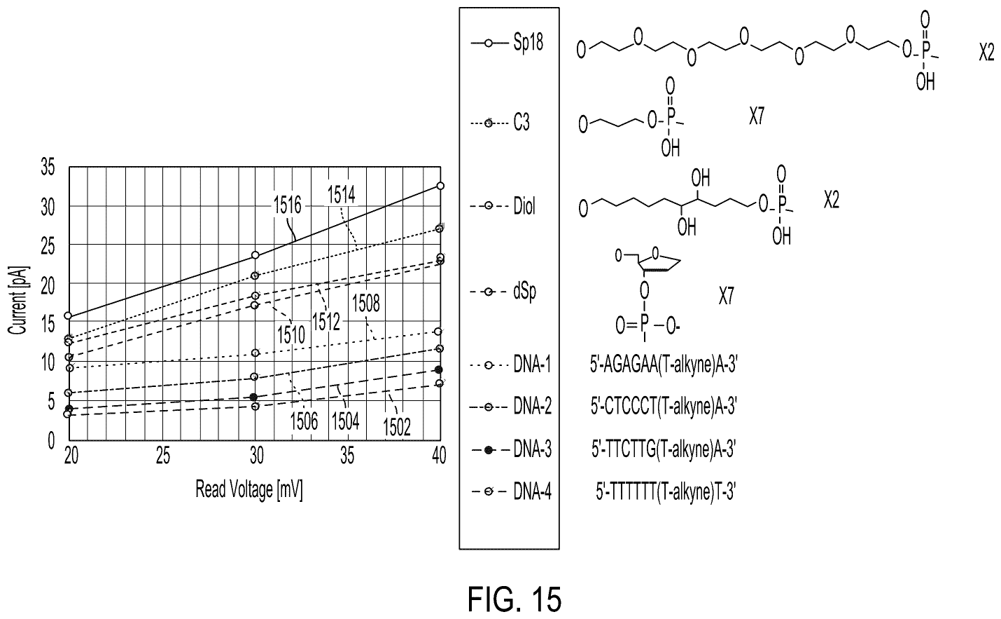

Eight candidate structures have been identified and will be discussed more with regard to FIG. 15, but a person having skill in the art would appreciate that other structures may be contemplated. Additionally, any of the structures or structure motifs below could be implemented in any of the reporting schemes shown in Table 1. In other words, any of the chemical structures below could be used to represent a 0, 1, 2, 3, 4, etc. in the encoding scheme of Table 1 (i.e. [0,1] is substituted for [Sp18, C3] or any number of consecutive Sp18's to represent “0” and any number of consecutive C3's to represent “1”).

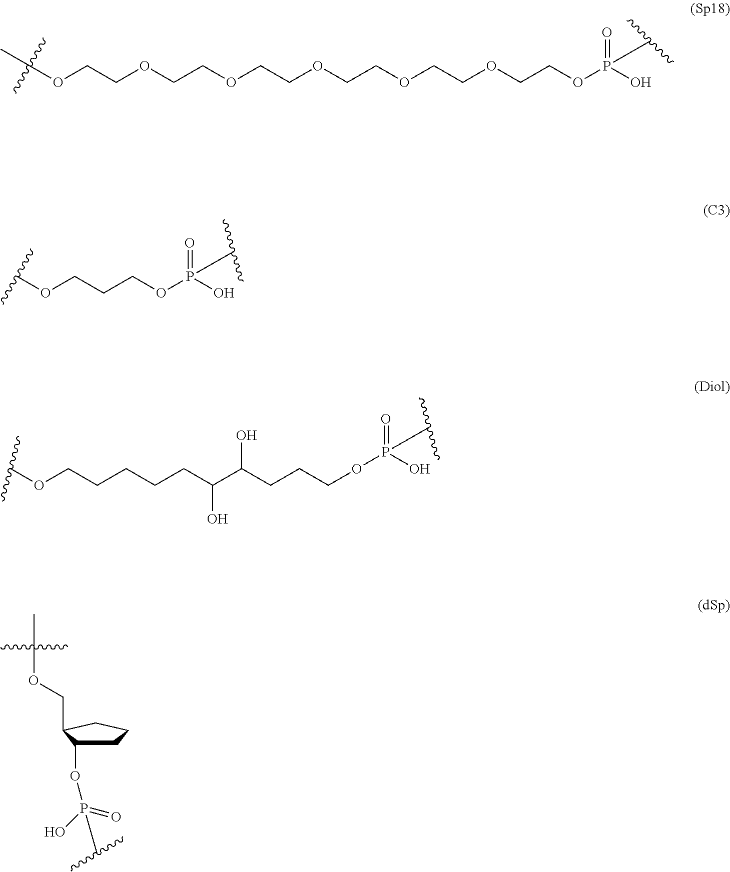

| | |
|---|---|
| 5′-A-G-A-G-A-A-(T-Alkyne)-A-3′ | DNA-1 |
| 5′-C-T-C-C-C-T-(T-Alkyne)-A-3′ | DNA-2 |
| 5′-T-T-C-T-T-G-(T-Alkyne)-A-3′ | DNA-3 |
| 5′-T-T-T-T-T-T-(T-Alkyne)-A-3′ | DNA-4 |

FIG. 15 shows the above candidate structures as a function of read voltage (in millivolts) on the x-axis and current (in picoamps) on the y-axis. DNA-4 is displayed as the lowest line 1502 followed by DNA-3 as the second lowest line 1503 followed by DNA-2 as the third lowest line 1506 followed by DNA-1 as the fourth lowest line 1508. The next line moving upward is to represent dSp (x7) as line 1510 followed by the next highest line 1512 to represent Diol (x2) followed by the next highest line 1514 to represent C3 (x7) and finally the highest line 1516 is to represent Sp18 (x2). Importantly, all of the potential reporters show a unique mean current signature (pA) across the potential read voltages from 20-40 mV. This is advantageous as each reporter provides a decipherable signal that may be used in the readout of a nucleobase. The configuration in FIG. 15 could be seen as corresponding with the lower current signals 806 and the higher current signals 804 in FIG. 8A. For example, lines 1502, 1504, 1506, and 1508 could be seen as generally corresponding with the lower current signals A, T, C, G and lines 1510, 1512, 1514, and 1516 could be seen as generally corresponding with the higher current signals A*, T*, C*, and G*. Advantageously, each of the potential reporters shown in FIG. 15 possesses a AI that is distinguishable from another reporter in FIG. 15.

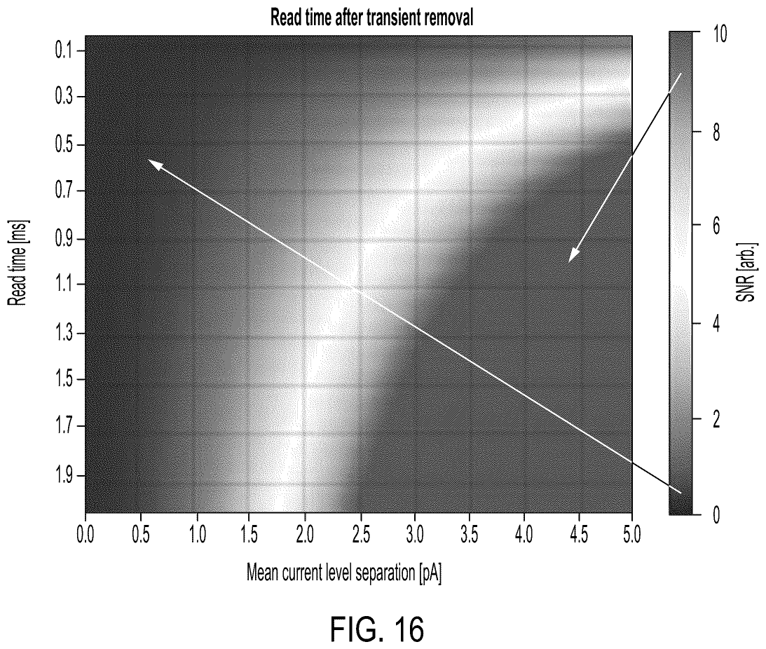

FIG. 16 depicts a theoretical graph of signal to noise ratio shown as a gray scale index with mean current level separation between adjacent reporters (in pA) on the x-axis and read time (in milliseconds) on the y-axis. As an example, the depiction in FIG. 16 could be applied to the mean current level separation for the candidate reporters in FIG. 15. To reduce or minimize mismatch error probabilities to less than 1 per 1,000, a minimum SNR of 5 is generally required. FIG. 16 generally depicts a SNR over 5 in the bottom right portion of the plot and a SNR under 5 in the upper left portion of the plot. Therefore, a person having ordinary skill in the art reading FIG. 16 would understand that it is advantageous to select current reporters that have about 2.5 pA of mean level separation between them. The advantages increase further with >2.5 pA mean current separation, as with greater separation (in pA) between the current reporters the read time (in milliseconds) decreases. In some examples it may be advantageous to select current reporters with separations greater than about 3 pA, greater than about 3.5 pA, greater than about 4 pA, or greater than about 4.5 pA. In some examples, it may be desirable to have sequencing of about 500 ARCs/sec (i.e., about 1 ms read times after transient removal of 1 ms). These calculations assume a basal current standard deviation of ±2 pA for samples collected at 10 kHz. In some examples it may be desirable to have read sequencing times of greater than about 550 ARCs/sec, greater than about 600 ARCs/sec, greater than about 650 ARCs/sec, or greater than about 700 ARCs/sec. Advantageously a faster read time is advantageous in decreasing overall costs. However, read times may be balanced with error correcting mechanisms that may slow down the nanopore readout but provide higher fidelity in the sequencing data. The plotted data in FIG. 16 are generally in line with the expected noise floor for nucleosidic and abasic reporters in various examples presented herein. In FIG. 16, and various other examples herein, the SNR may be calculated according to the following equation:

$$SNR = \frac{\vert {I}_{2} - {I}_{1} \vert}{\sqrt{Var ( {I}_{1} ) + Var ( {I}_{2} )}}$$

where I₂, I₁ are the mean reporter currents

### EC Reporter Variants (EC Oligomers)

As discussed above, an EC reporter may be used to encode a nucleobase, e.g., encoded into the cyclic loop of each nucleobase (e.g. implemented as an EC nucleotide). However, it may be advantageous in some instances to have a cyclic loop extend multiple nucleobases at a time (e.g. implemented as an EC oligomer (also referred to as an EC oligo)). Or a single cyclic loop may be bonded to a single nucleobase but encode for adjacent nucleobases at the same time. In such circumstances, the EC reporters may be configured to meet the various permutations of nucleobases, such as those discussed with respect to raw code. For example, with unmodified nucleobases the nanopore may read four bases at a time, which results in 4\hat{ }4=256 possible signals. Encoding an EC reporter for three bases at a time reduces this to 4\hat{ }3=64 different possible signals. Encoding an EC reporter for two bases at a time reduces this further to 4\hat{ }2=16 different possible signals. A person having ordinary skill in the art would likely balance the readout errors associated with each scheme with the speed of readout time, synthesis complexity, signal complexity, mean current distance between the various EC reporters, etc. Examples of cyclic loops having EC reporters that extend between two or more nucleobases are shown below in structure I to VI, where L₁ and L₂ are optional linkers and/or spacers:

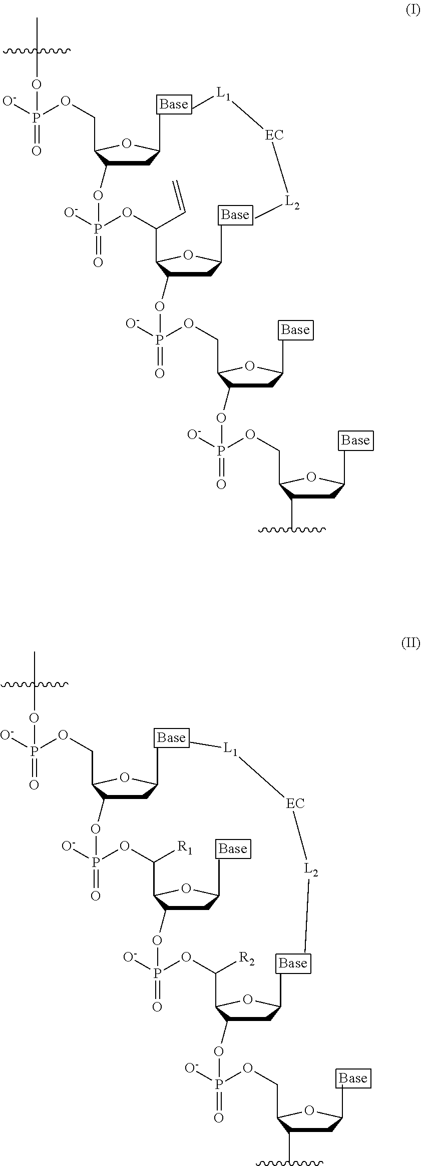

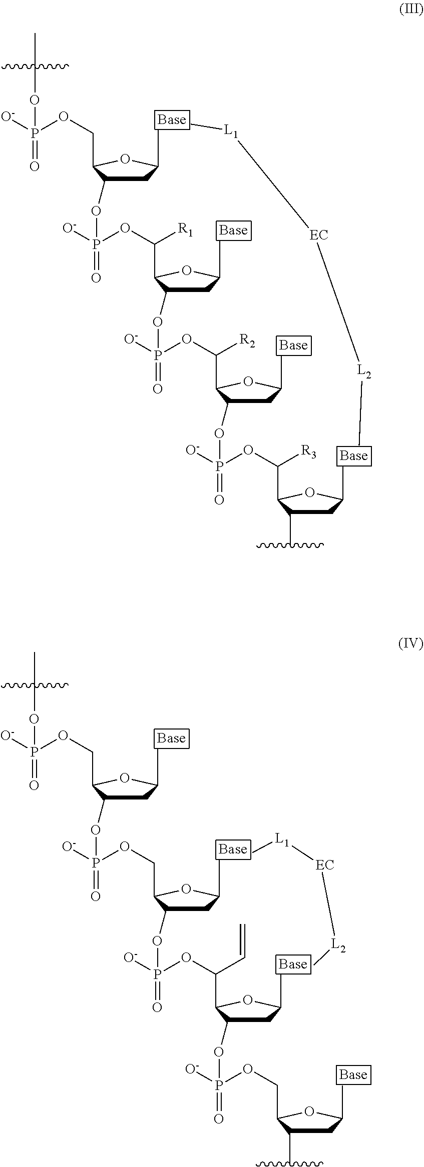

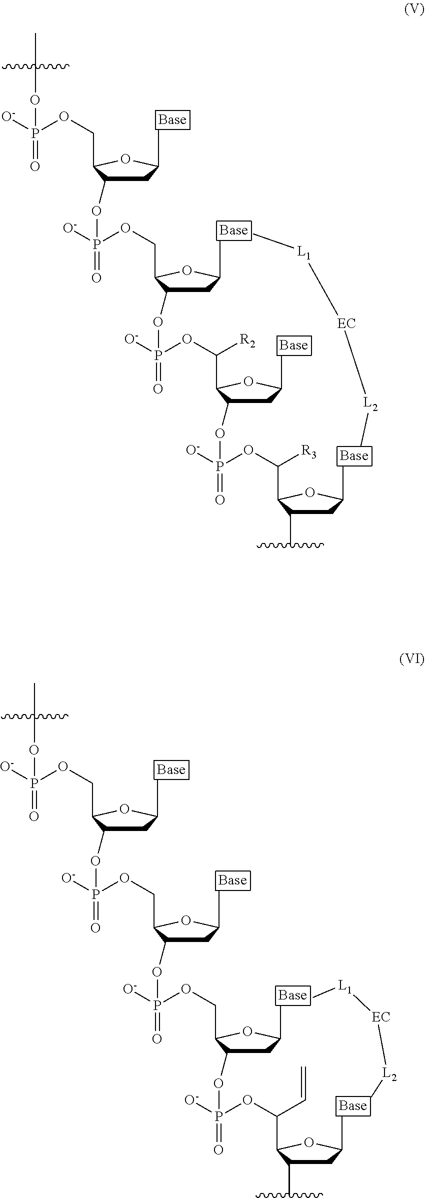

For the examples of cyclic loops having EC oligos (i.e. EC reporters that extend between two or more nucleobases) illustrated in structure I to VI, a polymer 400 may be synthesized using sequential ligation, where EC oligos such as I to VI may sequentially bind to their complementary single-stranded sample polynucleotide (e.g. such as DNA, RNA or otherwise), and the EC oligos may be joined together by a ligase. In some examples, elongation of the synthetic complementary strand may proceed by sequential ligation of the EC oligos to one another.

Many of the examples disclosed herein relating to the EC nucleotides (or EC oligos) have disclosed constant length encodings (where various encoding reporters in each scheme F-2-8 or F-5-5 employ the same number of signals in each cyclic loop). However, it may be advantageous to alter the number of arresting constructs, reporter elements, or spacers from one monomer unit (e.g., nucleobase) to another within the same scheme. Such a variable approach may be useful in applications where there may be specific sequence biases. For example, in organisms (or DNA strands) with relatively high GC content, it may be possible to create reporters for G and C that have fewer ARCs than reporters for A and T. This could result in a net overall speed-up in sequencing while maintaining error tolerance properties. A person having ordinary skill in the art would also appreciate that this variant approach may combine various aspects of the schemes discussed in Table 1 within the same polynucleotide daughter strand. Advantageously, variable length encodings may be better where there is self-synchronization, a process which inhibits or prevents errors downstream from where the error occurred due to added or deleted bits in a message. For example, variable length encodings of cyclic loops may be advantageous in self-synchronization in data with insertions and deletions.

Finally, the EC nucleotide examples disclosed herein may be advantageously compatible with an optical nanopore sensing strategy. For example, EC reporters may be particularly advantageous in optical nanopore sensing requiring few unique currents in the forward ratcheting mode, like the F-4-3 code in Table 1. As one non-limiting example, EC nucleotides may be used in optical nanopore sensing with calcium-sensitive fluorescent dyes coupled to a nanopore/lipid bilayer system to identify a set of unique reporters, or a smaller set of unique reporters.

### Additional EC Reporter Variants

Other examples of the EC-reporter may be incorporated or combined with the EC reporters disclosed herein. These can include extensions to ligation methods (as discussed elsewhere herein in the context of EC oligos) for generating the sequencing construct, encodings for use with alternating current waveforms, encodings for use with a combined optical nanopore system, and variable length encodings.

Alternating current waveforms allow to read an ARC in both the “forward” and “reverse” directions provided an ARC is flanked by two barcodes (i.e., current reporters on both sides). Switching between a “forward” and “reverse” read can be a robust indicator of transitions between sequencing template positions, and therefore allows to expand the possible space of EC-code designs. For example, it is possible to allow for some use of repeat current levels in the encoding (for example see Table 1, codes B-6-3 and B-4-2). The use of bipolar reads can also expand on the space of possible molecular construct designs. Some error correcting nucleotide examples can have EC-code words where the identity of a nucleotide is encoded with current signals (i.e. subreporters) positioned between two ARCs. However, it is also possible to design bipolar read compatible codes where subreporters are positioned flanking the ARCs. In EC reporting examples, the choice of waveform construct generally sets the constraints on EC-code design. However, alternating current waveforms can be configured consistent with any suitable combination of the following principles: First, in some examples, an EC nucleotide (or EC oligo) should maintain a minimum deletion distance between reporters in the encoding. The deletion distance is the number of signals that can be deleted in a reporter before the identity of the encoded base is lost. This is generally the basis of deletion error tolerance. Second, in some examples, an EC nucleotide (or EC oligo) should maintain a minimum insertion distance between reporters in the encoding. The insertion distance is the number of signal values that can be added to a reporter before the identity of the encoded base is lost. This is generally the basis of insertion error tolerance. Third, in some examples, an EC nucleotide (or EC oligo) should maintain a minimum Hamming distance between reporters in the encoding. The Hamming distance is the number of reporter signal values that can be changed before the identity of the encoded base is lost. This is generally the basis of mismatch error tolerance. Fourth, in some examples, an EC nucleotide (or EC oligo) should seek to increase or maximize the number of “higher current” to “lower current” transitions within each reporter, and across the set of all reporters. This lowers the requirements for the minimum signal to noise ratio (SNR). Fifth, in some examples, the EC nucleotide (or EC oligo) should seek to reduce or minimize the length of an encoding. This improves overall sequencing rates by requiring fewer ARCs (i.e., current readings) per nucleotide, as each ARC requires a minimum amount of sequencing time. Finally, in some examples, an EC nucleotide (or EC oligo) should seek to eliminate indistinguishable transitions between adjacent current reads. This removes needing foreknowledge of secondary metrics such as the mean duration of a current level to resolve transitions between ARCs.

### Transient Tick Mark Reporters

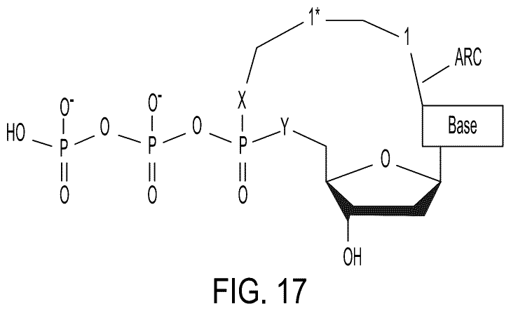

FIG. 17 illustrates a sample structure for a transient tick mark reporter. In FIG. 17 a cyclic loop connects the phosphate backbone to the nucleobase. The cyclic loop contains two reporter regions, represented by 1 and 1*. In FIG. 17, the 1* reporter may indicate the location of a universal transient tick mark, or nucleotide specific tick-mark; the 1 may indicate the location of candidate reporter structures used to identify the nucleobases (A, T (or U), C, G). Although not shown, any suitable one or more of various linkers or spacers may be included that can advantageously provide distance between successive reporter elements or between the reporter(s), tick mark(s), ARC(s), and the phosphate group(s) and/or the nucleobase.

The transient tick mark reporter scheme may be analogized to the F-2-8 EC scheme discussed above. However, the F-2-8 EC scheme places an arresting construct adjacent to (e.g., after) each of A*, T*, C*, or G*, whereas the transient tick mark scheme only incorporates an arresting construct in the monomer unit (e.g., cyclic loop). As such, the transient tick mark scheme provides the following non-exhaustive advantages. First, a single ARC is required per monomer unit (e.g., cyclic loop). Second, the transient tick mark may enable faster sequencing as compared EC schemes involving more than one ARC per monomer unit (e.g., cyclic loop). This is useful because, for example, two ARCs per cyclic loop effectively halves the number of pausing (stopping) sites in a 1× coverage scenario as compared to one ARC per reporter. Third, in the transient tick mark scheme the cyclic loop design is simpler and risk of scalability for manufacturing is lower. Fourth, the transient tick mark requires fewer number of pulses to translocate past each nucleotide (therefore resulting in faster raw sequencing rate per nucleotide) as compared to the EC nucleotides described elsewhere herein that have one ARC per reporter.

In some examples, a tick mark should be able to be placed between consecutive reporter elements, be observed after a voltage pulse that translocates the first arresting construct, and have a signal (fingerprint/signature) that differs substantially from the 4 chosen reporter signals used to encode the bases. However, as discussed above, using additional reporter signals and additional ARCs has the potential to slow down the readout process, whether because of increasing the number of ratchet events or increasing the distance between the nucleobases that are being read. As discussed above with respect to FIGS. 13A, 13B, and 13C, some EC reporting schemes may be advantageous because they may be run through a nanopore multiple times, reducing errors through multiple readings. On the other hand, an EC reporter scheme may be slower, but each read through the nanopore produces fewer read errors because of the additional reporters and/or ARCs included in that scheme (for example F-5-5), and fewer passes through the nanopore may be required in order to achieve a Q30 level.

Transient tick marks are advantageous as they do provide an additional level of redundancy compared to raw code (F-1-4), but have the same number of ARCs per cyclic loop as the raw code (compare FIG. 3 and FIG. 17, for example). The use of transient tick marks may advantageously reduce the percentage of basecalling error when compared to the raw (F-1-4) code, especially in homopolymer regions (e.g., where more than one nucleotide of similar type are adjacent to one another in the sample polynucleotide sequence).

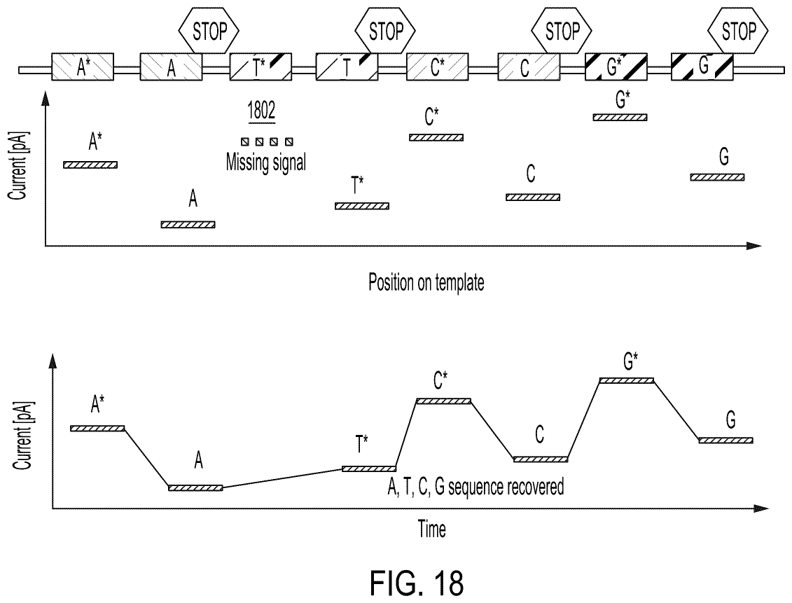

Furthermore, a transient tick mark scheme may be relatively tolerant in the event of random “deletion errors” where a reporter signal is unexpectedly absent. This is illustrated in FIG. 18, which shows the readout in the event of a deletion error 1802 in a polymer with tick marks (e.g., transient tick marks). Even though the T* signal was missing from the nanopore readout, the correct sequence of A-T-C-G was recovered. Further, the transient tick mark scheme is able to identify random deletion errors even at a lower depth of coverage.

### Transient Tick Mark Candidate Structures

Various structural units or repeating structural units may be incorporated into a cyclic loop in order to provide signals with relatively high fidelity. In some examples, transient tick marks should be able to be distinguishable from an ARC in the monomer unit (e.g., cyclic loop) and be able to translocate at a given read voltage without the need for a pulse voltage. Further, the transient tick mark should be able to provide a reproducible, distinguishable and resolvable signal from the reporter signals. Finally, the signal from the tick mark should generally be transient or short-lived but detectable a substantial fraction of the time (e.g., over 90%, over 95%, or over 99% depending on the desired deletion error accuracy that can be tolerated) as it translocates through the nanopore. Various chemical structures, such as PEG, aliphatic chains, synthetic polymers, polyphosphate or polypeptide, may operate as transient tick marks if they satisfy these general criteria.

The tick marks could be a single modification, or a plurality of modifications ranging anywhere from about 1-5, about 6-10, about 11-15, about 16-20, about 21-25, or about 26-50 unique and/or repeating subunits where they satisfy the three criteria listed above. Alternatively, or additionally, the tick marks could comprise macromolecules such as crown ether, cucurbituril, pillararenes, or cyclodextrins. For further details regarding crown ethers, see An et al., “Crown ether-electrolyte interactions permit nanopore detection of individual DNA abasic sites in single molecules,” PNAS 109 (29): 11504-11509 (2012), the entire contents of which are incorporated by reference herein. In some examples, conjugation of such macromolecules to the cyclic loop construct may be advantageously accomplished through covalent conjugation chemistries such as amine-NHS ester, amine-imidoester, amine-pentafluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, or azide-norbornene.

The four candidate structures below, which were also discussed with regard to FIG. 15, may be used alone or in combination with each other to provide a transient signal as they pass through a nanopore readhead.

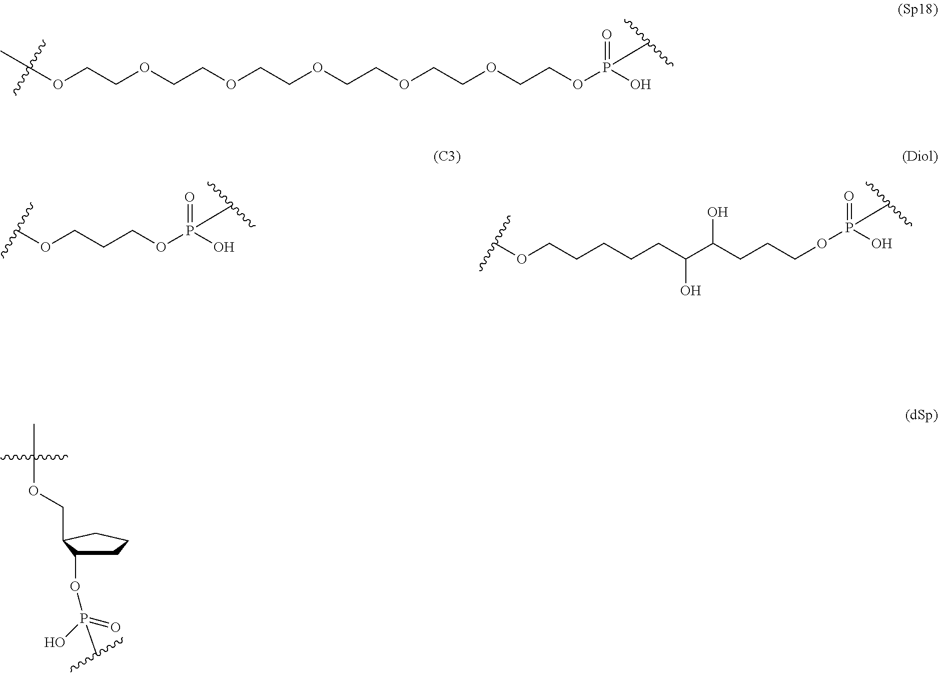

Further, these structures may be incorporated with additional chemical units such as T-Alkynes or other endcaps or spacers.

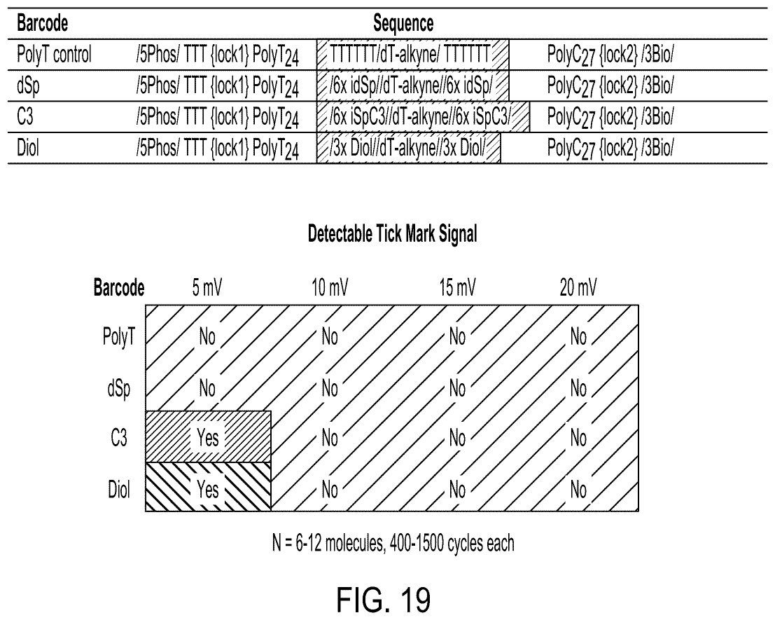

FIG. 19 illustrates data associated with various synthetic constructs for transient tick marks as compared to a baseline (control) signal of “TTTTTT/dT-alkyne/TTTTTT” (T=thymine nucleotide). As shown, the baseline signal did not provide a distinguishable signal from itself at 5, 10, 15, or 20 mV. The dSp structure (comprising six idSp units, a dT-alkyne unit in the middle, followed by six additional idSp units) also did not provide a distinguishable signal from the baseline at 5, 10, 15, or 20 mV. The C3 structure (six iSpC3 units, a dT-alkyne unit in the middle, followed by six additional iSpC3 units) provided a distinguishable signal from the baseline at 5 mV, but did not provide a distinguishable signal at 10, 15, or 20 mV. Finally, the Diol structure (three diol units, a dT-alkyne unit in the middle, followed by three additional diol units) provided the strongest distinguishable signal from the baseline at 5 mV, but did not provide a distinguishable signal at 10, 15, or 20 mV.

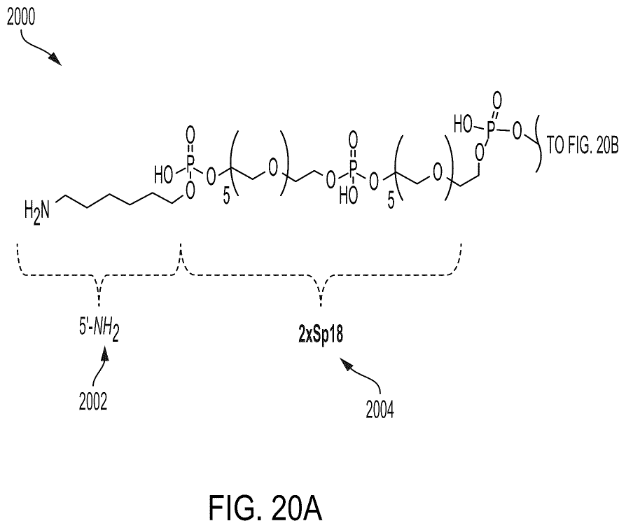

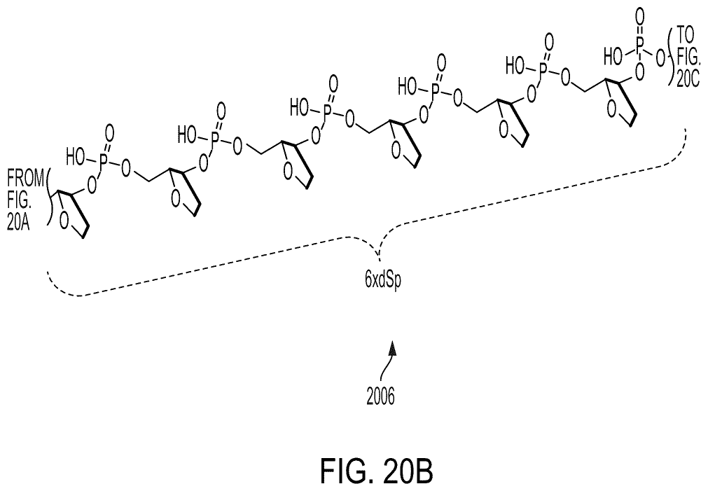

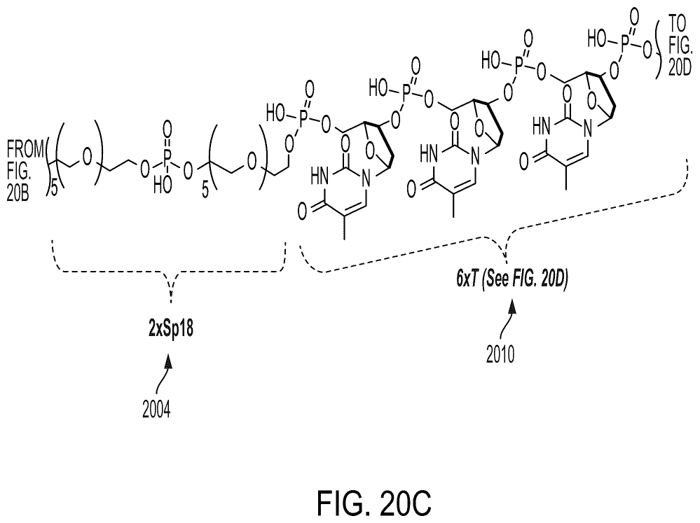

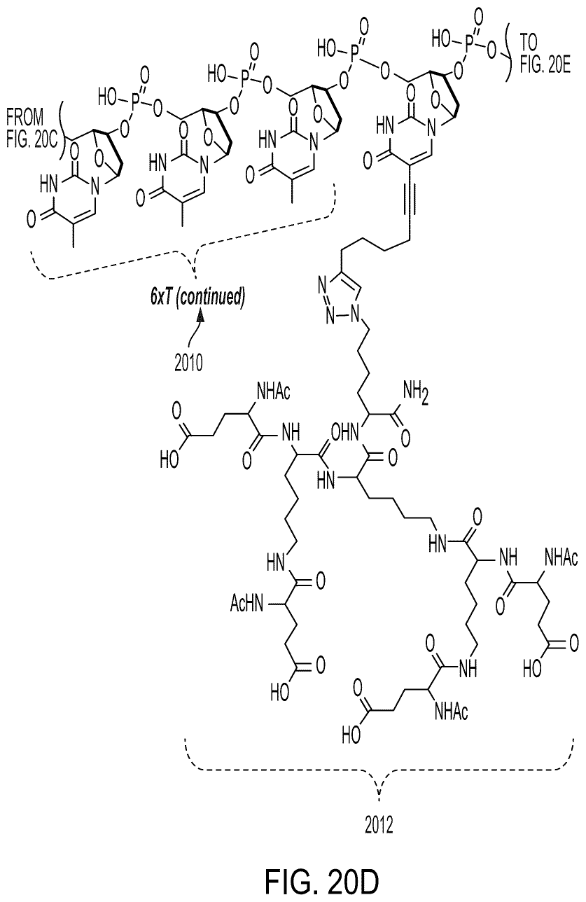

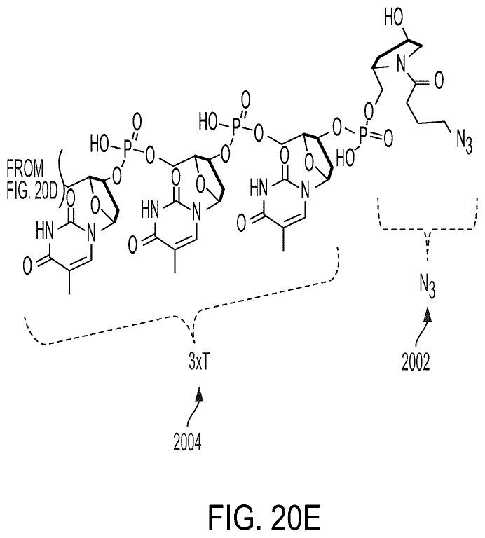

FIGS. 20A-20E depict an example of a sample cyclic loop structure 2000 with a transient tick mark comprising six dSp units. The 5′ end (FIG. 20A) begins with a conjugation or linker unit 2002 of NH₂ followed by two Sp18 spacers 2004. Following the spacers, the transient tick mark signal 2006 is provided (FIG. 20B), which is six repeating units of dSp. This is followed by another two Sp18 spacers 2004 (FIG. 20C). This is followed by an example reporter region 2010 (FIGS. 20C-20D), which may include six thymine base units. Thereafter an arresting construct (ARC) 2012 is provided (FIG. 20E). This ARC may operate as discussed with regard to various examples disclosed herein, so as to stop or slow the progression through the pore and generally requires a voltage bias to advance. Thereafter the cyclic loop is provided with three thymine nucleobases, which operate as spacers 2004. Finally, the structure ends with a conjugation or linker unit 2002 of N₃. The conjugation or linker units may allow for the cyclic loop to be attached to various points on a nucleotide, such as the nucleobase itself, a derivative of the nucleobase, or the phosphate backbone. Alternatively, the cyclic loop could span more than a single nucleotide, as discussed elsewhere herein. Finally, the transient tick mark in this example may be replaced by any of the structures with distinct signals discussed herein.

In some examples, the transient tick mark scheme is advantageous compared to the EC reporter schemes discussed herein as it provides a faster sequencing time, improved resolution for homopolymers and random deletion errors, and the cyclic loop design is synthetically simpler with fewer unique reporters and/or spacers and/or tick marks. As discussed above, in some examples, the transient tick mark scheme is advantageous as it may provide at least a 10-fold error reduction over raw code.

### Cyclic Loop Units

In various examples, a chemical subunit may be employed as a spacer or a reporter, depending upon its repeating structure within a string of modified nucleotides or a cyclic loop. The example structures below may be used as spacers or reporters, such as nucleobase reporters or as transient tick marks.

The following depicts various polyphosphate subunits (where “a” indicates a repeating subunit and X may be O⁻, OMe, or S⁻). Each of these may be used as spacers or signal units, such as reporters or tick marks:

### Phosphate Units

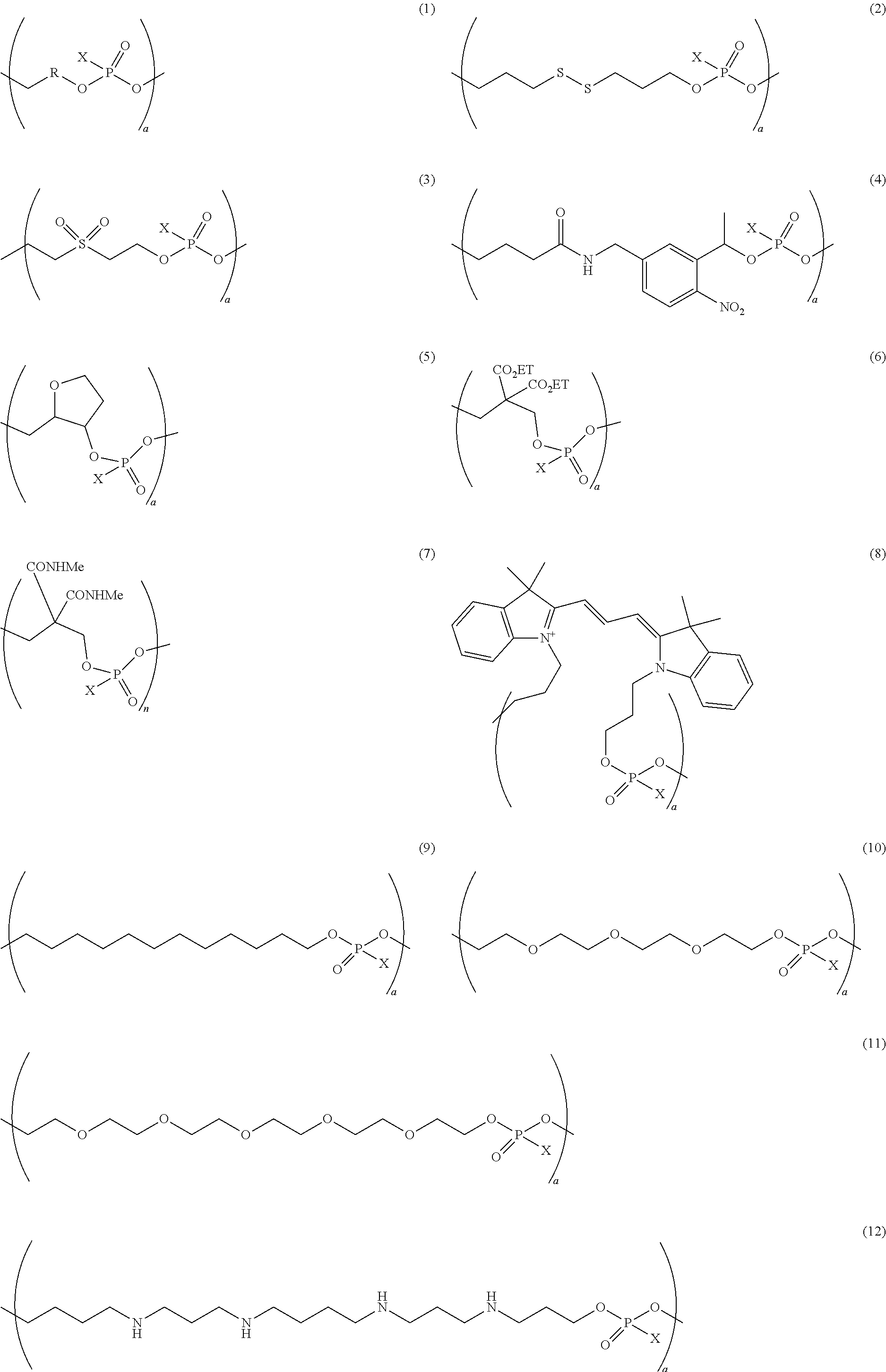

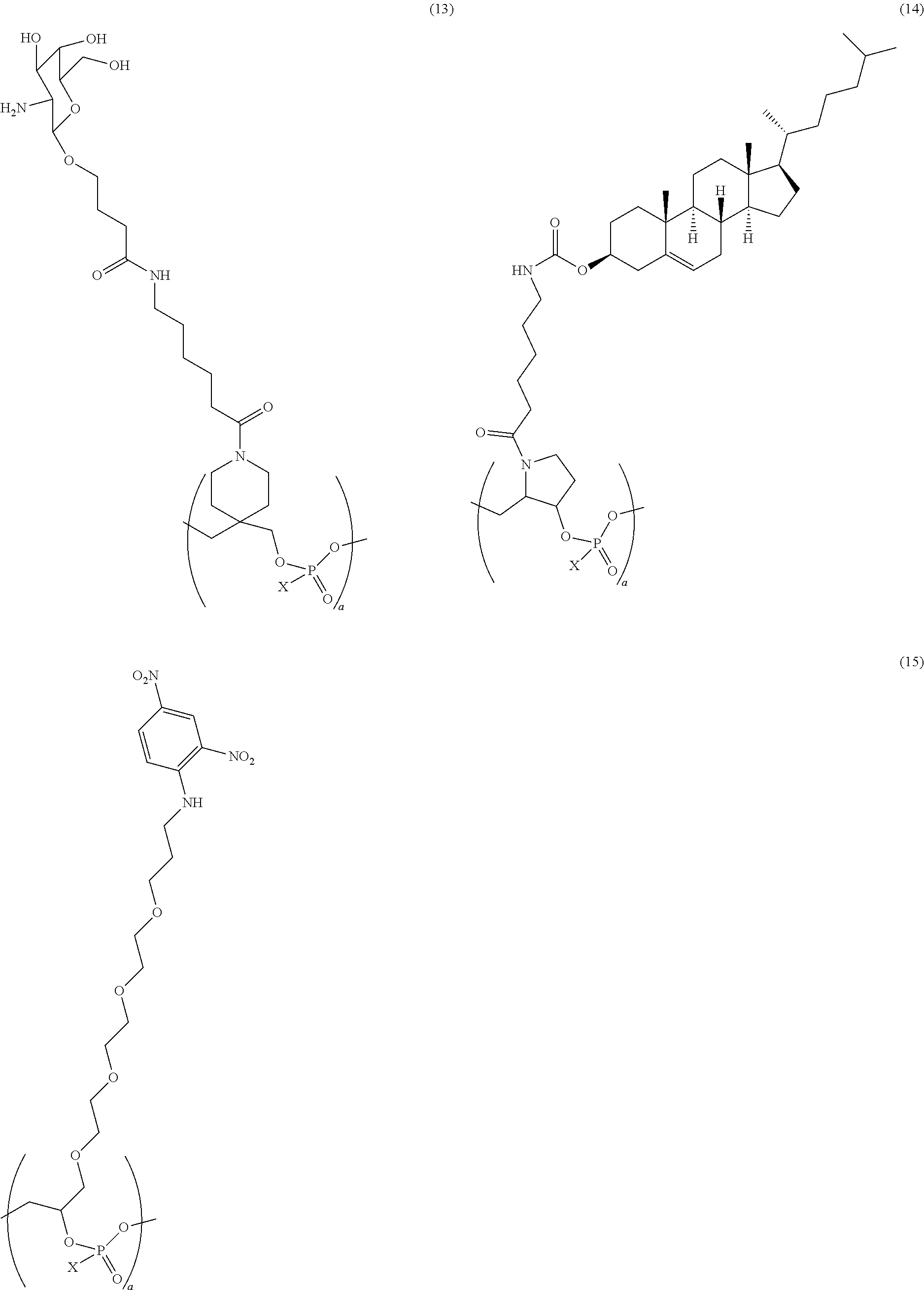

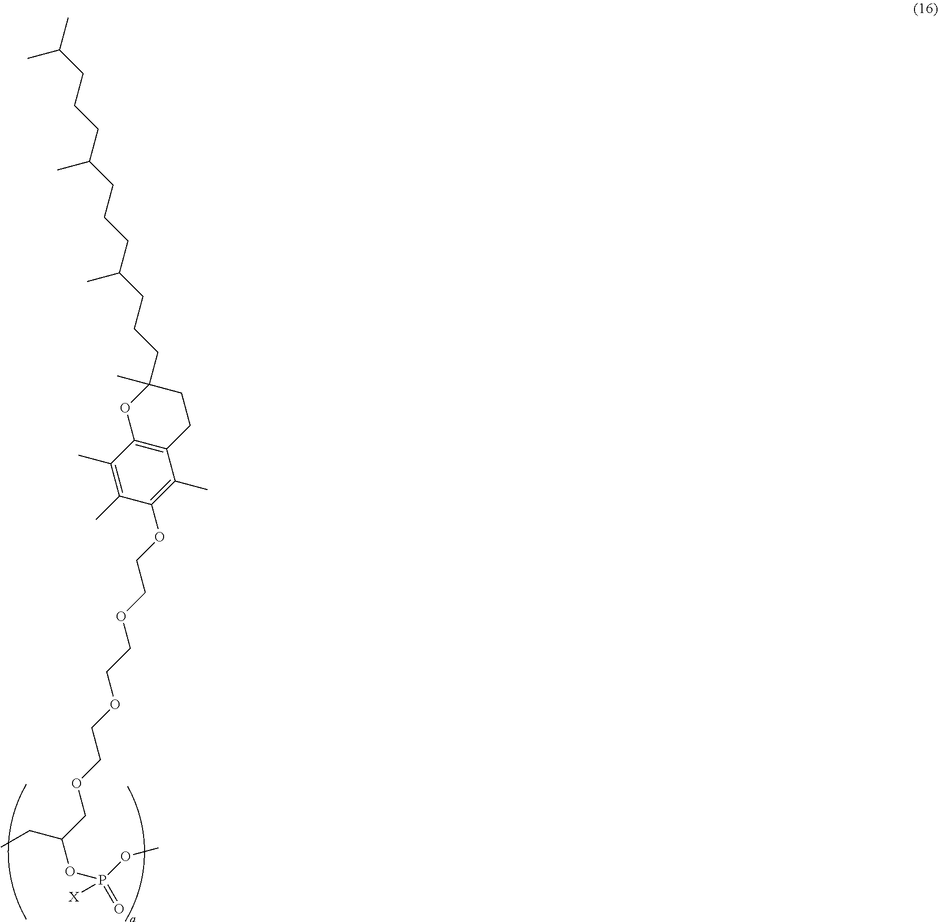

The following depicts various polyamide subunits (where “n” or “m” indicates a repeating subunit and is a positive integer). The polyamide may be a homopolyamide (comprising a single type of amide subunit in sequence, such as shown below) or a heteropolyamide (comprising a mixture of different types of amide subunits). Each of these may be used as spacers or signal units, such as reporters or tick marks:

### Polyamide Units

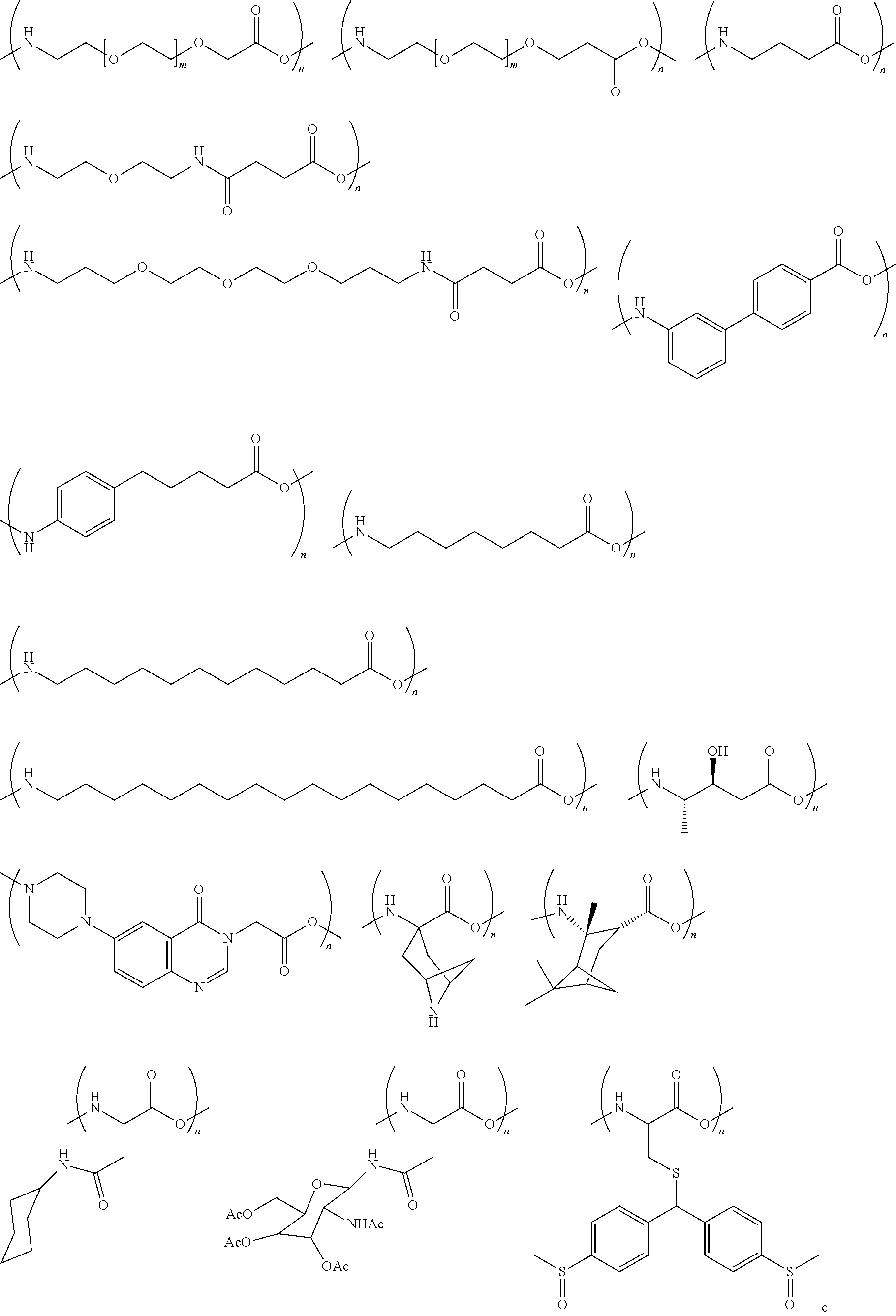

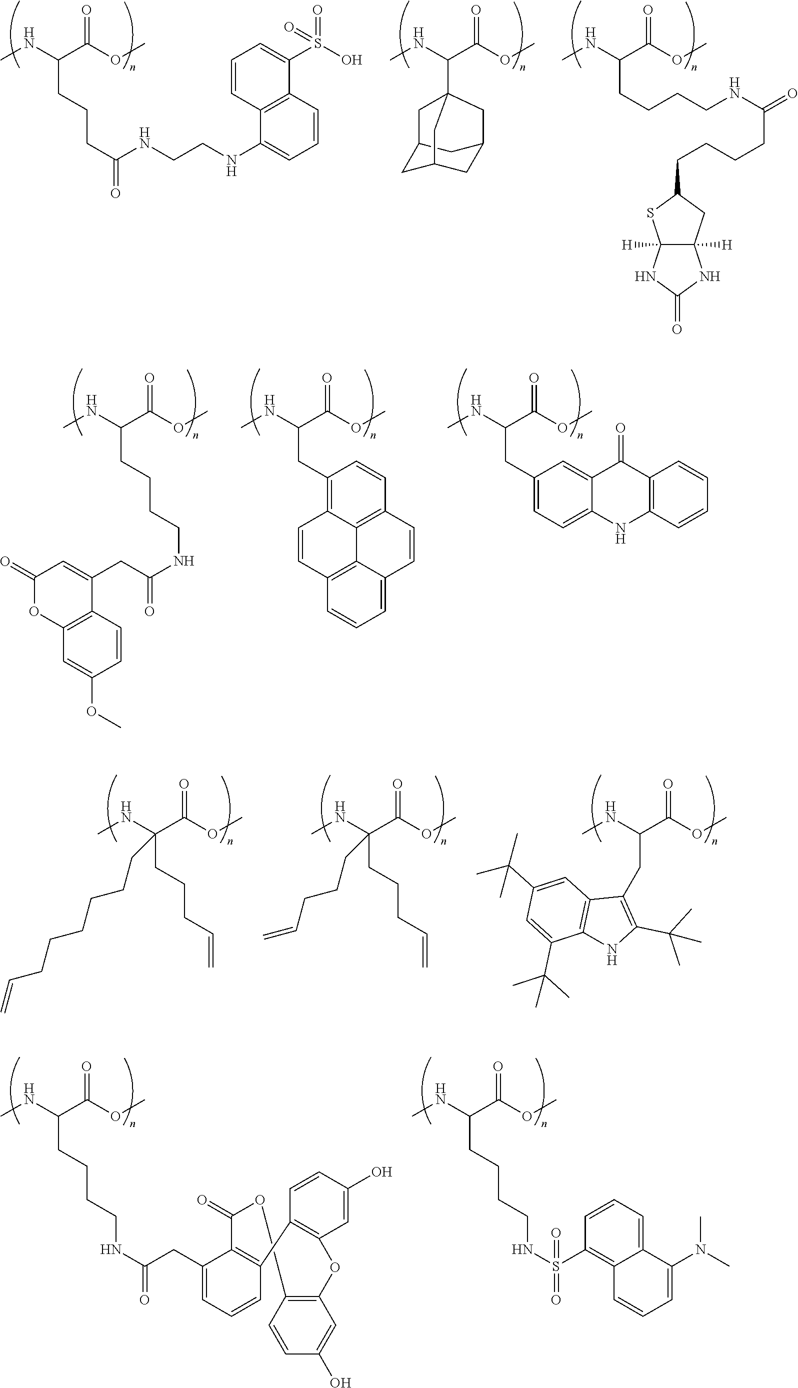

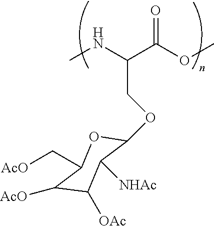

The following depicts various synthetic polymer subunits (where “n” indicates a repeating subunit that is a positive integer). Each of these may be used as spacers or signal units, such as reporters or tick marks:

### Synthetic Polymer Units

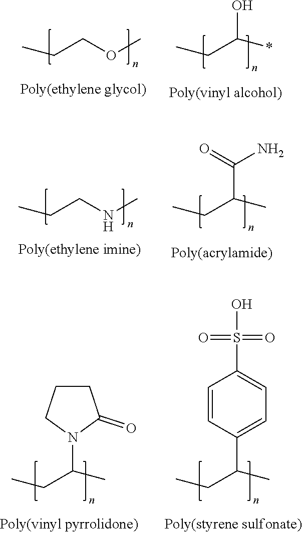

The following depicts various peptide subunits (where “n” indicates a repeating subunit that is a positive integer). The peptide may be a homopolypeptide (comprising a single type of peptide subunits in sequence) or a heteropolypeptide (comprising a mixture of different types of peptide subunits in sequence). The peptide sequence may comprise any combination of natural L or D amino acids or unnatural (non-naturally occurring) L or D amino acids. Each of the following structures may be used as spacers or signal units, such as reporters or tick marks:

### Peptide Units

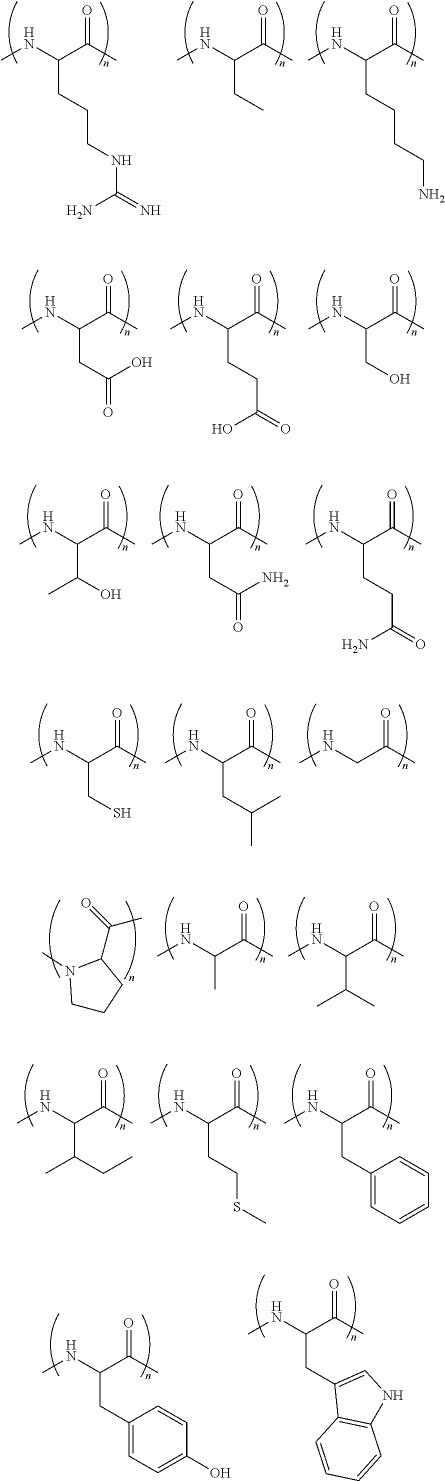

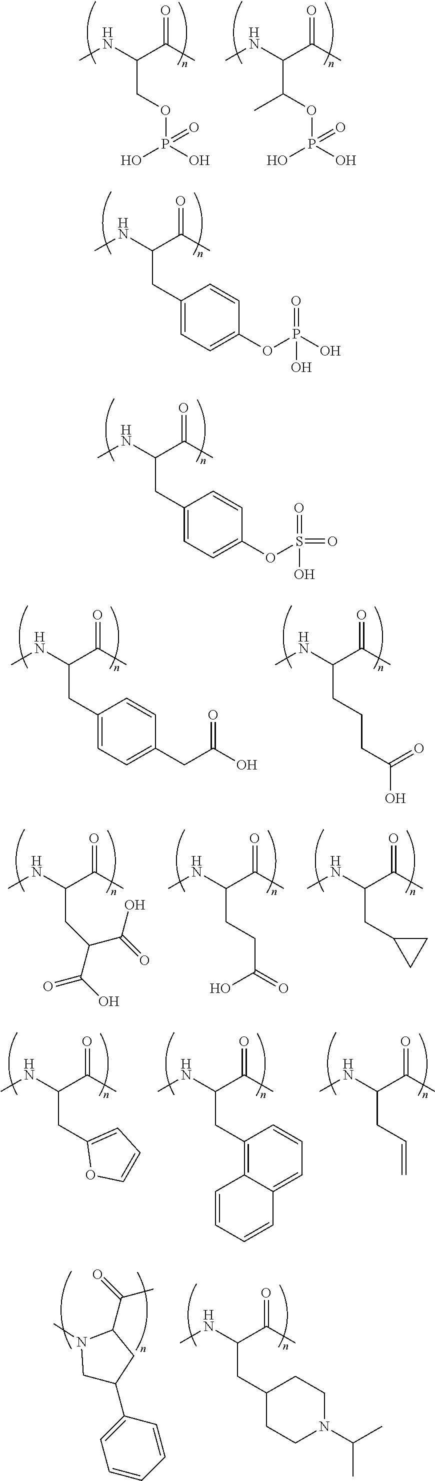

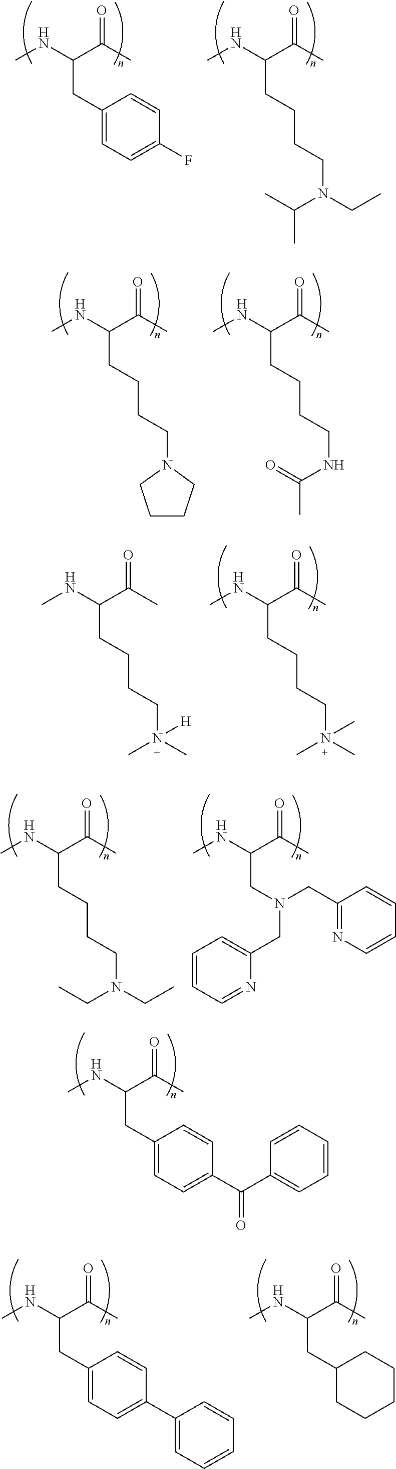

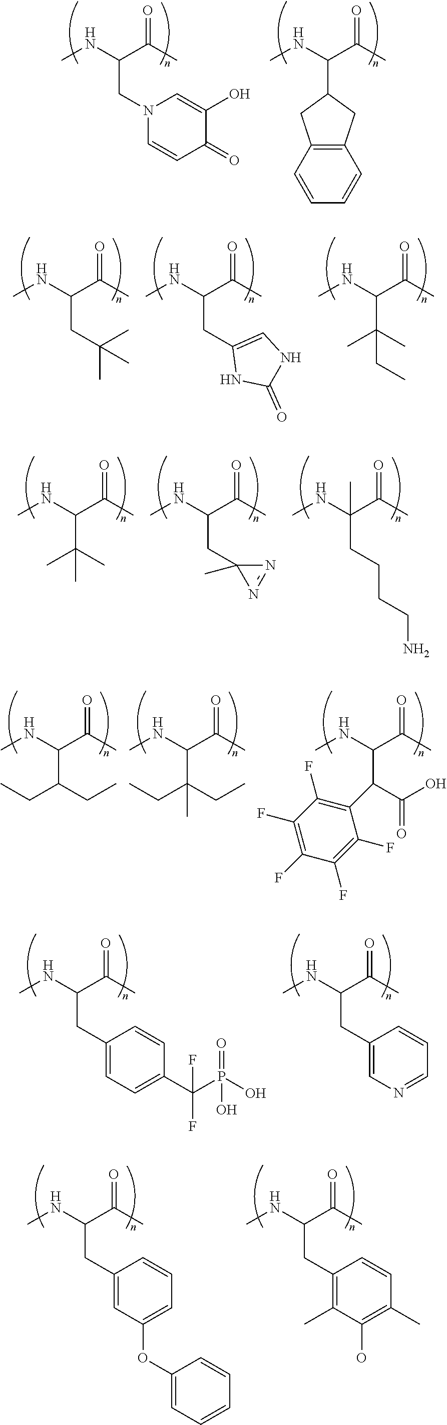

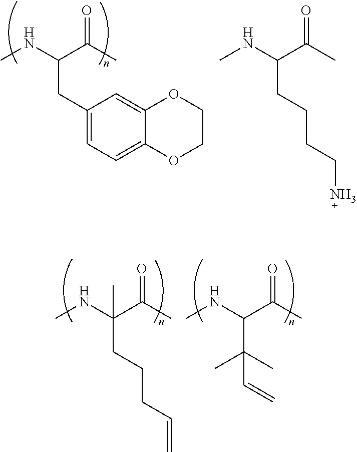

### Example Sequencing Methods, Polymers, Compositions, and Devices

From the foregoing, it will be appreciated that a wide variety of polymers may be used to encode the sequences of polynucleotides, and that a wide variety of nanopore sequencing methods may be implemented to determine such sequences.

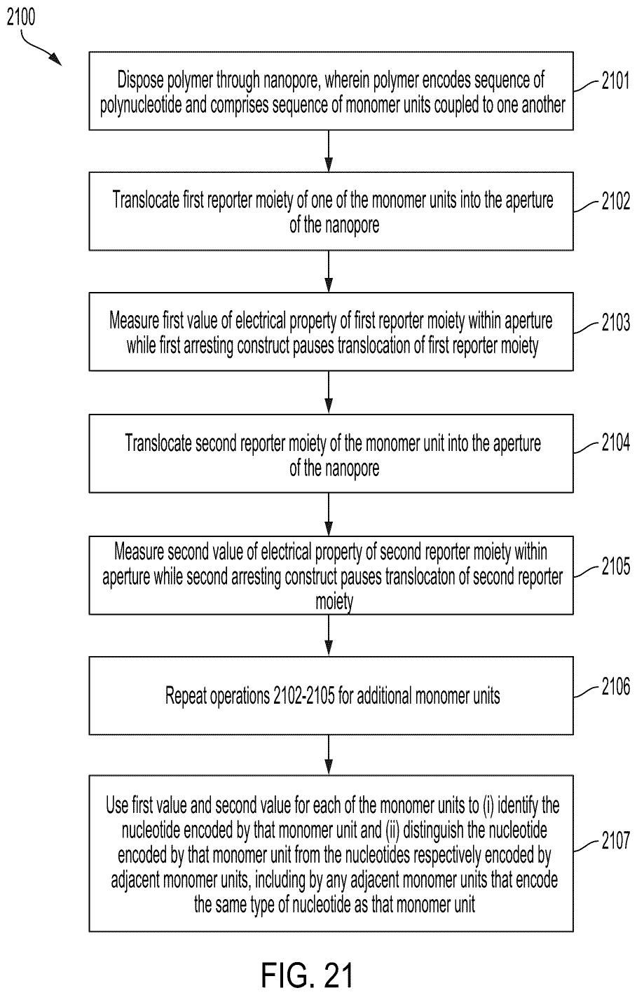

For example, FIG. 21 illustrates an example flow of operations in a method for sequencing a polynucleotide using a polymer that encodes a sequence of the polynucleotide. Method 2100 illustrated in FIG. 21 may include disposing a polymer through a nanopore, wherein the polymer encodes a sequence of a polynucleotide and comprises a sequence of monomer units coupled to one another (operation 2101). The nanopore may have a first side, a second side, and an aperture extending through the first and second sides, and the polymer may be disposed through the aperture such that a first end of the polymer is on the first side of the nanopore, and a second end of the polymer is on the second side of the nanopore, for example in a manner such as described with reference to FIG. 1A.

Each of the monomer units may encode an identity of a nucleotide in the polynucleotide. In some examples, each of the monomer units may include a first reporter moiety; a second reporter moiety; a first arresting construct; and a second arresting construct. Nonlimiting examples of such monomer units are the F-2-5, F-2-4, F-2-8, F-3-4, F-4-3, F-4-4, F-5-3, F-5-5, B-6-3, and B-6-4 EC codes described with reference to Table 1 and FIGS. 5, 6B, 7, 8A-8B, 9A-9B, 10A-10B, 11, 12, 13A-13C, 14, and 18. Nonlimiting examples of reporter moieties and arresting constructs suitable for use in the monomer units are described elsewhere herein.

Referring again to FIG. 21, method 2100 may include translocating the first reporter moiety of one of the monomer units into the aperture of the nanopore (operation 2102). For example, in a manner such as described with reference to FIG. 1A, a voltage bias may be applied across the nanopore that causes the polymer to move relative to the nanopore. In some examples, the voltage bias may cause the polymer to move towards the first side of the nanopore, such that the first reporter moiety enters the aperture from the second side of the nanopore (such an example may in some circumstances be referred to herein as a “reverse” read). In other examples, the voltage bias may cause the polymer to move towards the second side of the nanopore, such that the first reporter moiety enters the aperture from the first side of the nanopore (such an example may in some circumstances be referred to herein as a “forward” read).

Method 2100 illustrated in FIG. 21 further may include measuring a first value of an electrical property of the first reporter moiety within the aperture while the first arresting construct pauses translocation of the first reporter moiety (operation 2103). For example, in a manner such as described further above, the arresting construct may stop or slow translocation of the first reporter moiety out of the aperture, thus providing additional time to obtain a signal from the first reporter moiety.

Method 2100 illustrated in FIG. 21 further may include translocating the second reporter moiety of the monomer unit into the aperture (operation 2104). Method 2100 further may include measuring a second value of an electrical property of the second reporter moiety within the aperture while the second arresting construct pauses translocation of the second reporter moiety (operation 2105). In some examples, translocating the first reporter moiety of that monomer unit into the aperture includes applying a first stimulus, and translocating the second reporter moiety of that monomer unit into the aperture includes applying a second stimulus. The second stimulus may be applied at a different time than the first stimulus. Additionally, or alternatively, the first stimulus and the second stimulus may be of substantially the same magnitude as one another. Illustratively, a first voltage bias may be applied during operation 2102, a second voltage bias may be applied during operation 2103, a third voltage bias may be applied during operation 2104, and a fourth voltage bias may be applied during operation 2105. In some examples, the first and third voltage biases may have the same magnitude as one another but are at different times than one another, and the second and fourth voltage biases may have the same magnitude as one another but are at different times than one another. In other examples, the first reporter moiety is translocated out of the aperture under a constant a stimulus, and the second reporter moiety is translocated out of the aperture under the constant stimulus. Illustratively, the same voltage bias as described with reference to operation 2102 may be continuously applied across the nanopore throughout operations 2102 through 2104 that causes the polymer to move relative to the nanopore in a manner which is modulated by the first and second arresting constructs (e.g., in a scheme which may be referred to above as “auto-ratchet”).

Method 2100 illustrated in FIG. 21 further may include repeating operations 2102 through 2105 for additional monomer units (operation 2106). For example, the voltage bias(es) may be continually or repeatedly applied across the nanopore to obtain values for the electrical properties of the reporter moieties of different monomer units in the polymer. The signals obtained during operations 2103, 2105 (and repetitions thereof) may characterize any suitable electrical property of the first and second reporter moieties. For example, measuring the first value during operation 2103 may include characterizing a first electrical current, ionic current, electrical resistance, or electrical voltage drop across the nanopore while the first reporter moiety is within the aperture; and measuring the second value during operation 2105 may include characterizing a second electrical current, ionic current, electrical resistance, or electrical voltage drop across the nanopore while the second reporter moiety is within the aperture.

Method 2100 illustrated in FIG. 21 further may include using the first value and the second value for each of the monomer units to (i) identify the nucleotide encoded by that monomer unit; and (ii) distinguish the nucleotide encoded by that monomer unit from the nucleotides respectively encoded by adjacent monomer units, including by any adjacent monomer units that encode the same type of nucleotide as that monomer unit (operation 2107).

Nonlimiting examples of the manner in which respective signals from first and second reporters of a given monomer unit (e.g., cyclic loop) may be used to identify nucleotides and to distinguish nucleotides from one another (even in a homopolymer region) are described with reference to FIGS. 6B, 7, 8A-8B, 9A-9B, 10A-10B, 13A-13C, and 18. Illustratively, in some examples, the first reporter moiety uniquely identifies the nucleotide encoded by that monomer unit and the second reporter moiety does not uniquely identify the nucleotide encoded by that monomer unit, or the second reporter moiety uniquely identifies the nucleotide encoded by that monomer unit and the first reporter moiety does not uniquely identify the nucleotide encoded by that monomer unit. In some such examples, the reporter moiety that does not uniquely identify the nucleotide encoded by that monomer unit may be or include a tick mark (e.g., in an F-2-5 scheme such as described with reference to Table 1).

In other examples, the first reporter moiety uniquely identifies the nucleotide encoded by that monomer unit; and the second reporter moiety uniquely identifies the nucleotide encoded by that monomer unit (e.g., in an F-2-8 scheme such as described with reference to Table 1). In a manner such as described above, such a scheme may be particularly robust to deletion errors. For example, in such a scheme, for an additional one of the monomer units, a single deletion error causes the first value to be measured and the second value not to be measured, or causes the first value not to be measured and the second value to be measured. Nonetheless, the measured first value may be used to uniquely identify the nucleotide and the non-measurement of the second value to identify that the single deletion error occurred; or the measured second value may be used to uniquely identify the nucleotide and the non-measurement of the first value to identify that the single deletion error occurred, for example in a manner such as described with reference to FIGS. 9A-9B. Additionally, or alternatively, in such a scheme, for an additional one of the monomer units, a single insertion error causes the first value to be measured more than once, or causes the second value to be measured more than once. The method may include using the twice measured first value to uniquely identify the nucleotide and to identify that the single insertion error occurred; or using the twice measured second value to uniquely identify the nucleotide to identify that the single insertion error occurred.

In still other examples, the first and second reporter moieties, alone, do not uniquely identify the nucleotide encoded by that monomer unit; and the first and second reporter moieties, together, uniquely identify the nucleotide encoded by that monomer unit, for example in a manner such as described above for the F-2-4, F-3-4, F-4-3, F-4-4, F-5-3, F-5-5, B-6-3, and B-6-4 schemes with reference to Table 1.

For example, each of the monomer units may further include a third reporter moiety and a third arresting construct, where the first, second, and third reporter moieties, alone, do not uniquely identify the nucleotide encoded by that monomer unit; and the first, second, and third reporter moieties, together, uniquely identify the nucleotide encoded by that monomer unit, for example in a manner such as described above for the F-3-4 scheme with reference to Table 1.

As another example, each of the monomer units further may further include a third reporter moiety, a fourth reporter moiety, and a third arresting construct, where the first, second, third, and fourth reporter moieties, alone, do not uniquely identify the nucleotide encoded by that monomer unit; and the first, second, third, and fourth reporter moieties, together, uniquely identify the nucleotide encoded by that monomer unit, for example in a manner such as described above for the F-4-3, F-4-4, F-5-3, F-5-5, B-6-3, and B-6-4 schemes with reference to Table 1.

As yet another example, each of the monomer units further may include a fifth reporter moiety, a fourth arresting construct, and a fifth arresting construct, wherein the fifth reporter moiety does not uniquely identify the nucleotide encoded by that monomer unit, for example in a manner such as described above for the F-5-3 and F-5-5 schemes with reference to Table 1.

In some examples, the method further includes, before repeating operations 2102-2105 for additional monomer units, translocating the second reporter moiety of that monomer unit out of the aperture while translocating that first reporter moiety into the aperture, and measuring a third value of an electrical property of that first reporter moiety within the aperture while the second arresting construct pauses translocation of that first reporter moiety. The method further may include using the first, second, and third values together to: identify the nucleotide encoded by that monomer unit; and distinguish the nucleotide encoded by that monomer unit from the nucleotides respectively encoded by adjacent monomer units, including by any adjacent monomer units that encode the same type of nucleotide as that monomer unit. Such an example may be described elsewhere herein as a bipolar measurement which includes forward and backward translocation through alternating forward and reverse currents, e.g., on a monomer-unit by monomer-unit basis, or even on the basis of a portion of the monomer unit (e.g., on a reporter-region by reporter-region basis). This may be thought of as “flossing” the monomer unit or a portion thereof.

In some examples, the method further includes, after repeating operations 2102-2105 for additional monomer units, translocating a plurality of the monomer units to the first side of the nanopore, and then repeating operations 2102 through 2105 to obtain a plurality of additional values characterizing the polynucleotide encoded by the polymer. This may be thought of as “flossing” a portion of the polymer that contains multiple monomer units. Each such monomer unit (or portion thereof) may be individually “flossed” as well in a manner such as described elsewhere herein.

From the present disclosure, it will be apparent that within a given monomer unit (e.g., within a given cyclic loop) the arresting construct(s) and reporter moiet(ies) may have any suitable arrangement relative to one another and relative to other portions of that monomer unit.

In some examples, the first arresting construct may be disposed between the first reporter moiety and the second reporter moiety of an adjacent monomer unit. Alternatively, the first arresting construct may be disposed between the first reporter moiety and the nucleobase. Additionally, or alternatively, in some example the second arresting construct may be disposed between the first reporter moiety and the second reporter moiety. Nonlimiting examples of various such arrangements are described with reference to FIGS. 5, 6A-6B, 7, 8A-8B, 9A-9B, 11, 12, and 14.

Additionally, from the present disclosure, it will be apparent that operations such as described with reference to FIG. 21 may be performed in any suitable order, and are not limited to the order specifically suggested in FIG. 21. For example, operations 2102 and 2103 may be performed before operations 2104 and 2105. Or, for example, operations 2102 and 2103 may be performed after operations 2104 and 2105. That is, reference herein to a given reporter moiety, arresting construct, monomer unit, or other element or operation being “first,” “second,” “third,” or the like is for convenience of distinguishing such elements or operation from one another, rather than intending to connote any specific ordering in space or time of such element or operation.

It will further be appreciated that the present polymers may be used in any suitable system, apparatus, device, method, or operation, and are not limited to use in methods such as described herein. For example, an optical readout may be used to characterize changes in the ionic current through a nanopore which are caused by reporter moieties having different electrical characteristics than one another. In some examples, a polymer encoding a sequence of a polynucleotide may include a sequence of monomer units coupled to one another. Each of the monomer units encoding an identity of a nucleotide in the polynucleotide and may include a first reporter moiety; a second reporter moiety; a first arresting construct; and a second arresting construct. Nonlimiting examples of such monomer units are the F-2-5, F-2-4, F-2-8, F-3-4, F-4-3, F-4-4, F-5-3, F-5-5, B-6-3, and B-6-4 EC codes described with reference to Table 1 and FIGS. 5, 6B, 7, 8A-8B, 9A-9B, 10A-10B, 11, 12, 13A-13C, 14, and 18. In other examples, each of the monomer units may include a first reporter moiety; a second reporter moiety having a different signal characteristic (e.g. electrical property, optical property, or otherwise) than the first reporter moiety; and a first arresting construct. Such monomer units optionally can, but need not necessarily, include a second arresting construct. Nonlimiting examples of such monomer units are the are the F-1-4, F-2-5, F-2-4, F-2-8, F-3-4, F-4-3, F-4-4, F-5-3, F-5-5, B-6-3, and B-6-4 EC codes described with reference to Table 1 and FIGS. 5, 6B, 7, 8A-8B, 9A-9B, 10A-10B, 11, 12, 13A-13C, 14, 17, and 18. Nonlimiting examples of reporter moieties and arresting constructs suitable for use in any such monomer units are described elsewhere herein.

In some examples, the polymer includes four different types of the monomer units.

Illustratively, a first type of the monomer units corresponds to a first type of nucleotide (e.g., A); a second type of the monomer units corresponds to a second type of nucleotide (e.g., C); a third type of the monomer units corresponds to a third type of nucleotide (e.g., G); and a fourth type of the monomer units corresponds to a fourth type of nucleotide (e.g., T or U). In some examples, the first reporter moieties of the first, second, third, and fourth types of the monomer units are of different types than one another. Additionally, or alternatively, in some examples, at least some of the second reporter moieties of the first, second, third, and fourth types of the monomer unit are of the same type as one another, for example, in EC coding configurations. Alternatively, in some examples, the second reporter moieties of the first, second, third, and fourth types of the monomer unit are of the same type as one another, for example, as in TM configurations.

Similarly as discussed with reference to method 2100, although the present polymers are not limited to use with such a method, each of the monomer units further may include a third reporter moiety; and a third arresting construct, for example such as in the F-3-4 and higher order EC codes and the B-6-4 and other bipolar (B) codes described with reference to Table 1. In some examples, each of the monomer units further includes a fourth reporter moiety, for example such as described with reference to the cyclic loop of FIG. 12 or FIG. 14. In some examples, within each one of the monomer units, at least two of the first, second, third, and fourth reporter moieties within that monomer unit are of different types than one another. For example, at least three of the first, second, third, and fourth reporter moieties within that monomer unit may be of different types than one another. Or, for example, all four of the first, second, third, and fourth reporter moieties within that monomer unit may be of different types than one another. A wide variety of options for the number of different types of reporter moieties that may be used in a given monomer unit are described with reference to the various codes of Table 1. Such reporter moieties may have any suitable arrangement relative to one another. Illustratively, the third arresting construct, the third reporter moiety, and the fourth reporter moiety may be disposed between the first reporter moiety and the second reporter moiety. Other arrangements suitably may be used.

It will further be apparent that the reporter moieties of the present polymers, regardless of the particular method(s) in which such polymers are used, may have any suitable characteristics that generate signals which are sufficiently distinguishable from one another. Illustratively, the first reporter moiety may have a first electrical characteristic, and the second reporter moiety may have a second electrical characteristic that is different from the first electrical characteristic. Similarly, any other reporter moit(ies) in the present polymers may have electrical characteristics that are different than—or the same as, in some examples—one another. Such characteristics may facilitate distinguishing the reporter moieties from one another and/or may facilitate distinguishing monomer units from one another, e.g., so as to solve the homopolymer problem as well as the problem of deletion errors, insertion errors, and/or mismatch errors.

In nonlimiting examples in which the present polymers are used with a nanopore, the present polymers are compatible with any suitable arresting constructs that may spontaneously translocate through a nanopore or may translocate through a nanopore responsive to a stimulus (e.g., appropriate voltage bias across the nanopore). In some examples, the first arresting construct may be of the same type as the second arresting construct. In other examples, the first arresting construct may be of a different type than the second arresting construct. Illustratively, the first arresting construct may include a peptide. The second arresting construct may include peptide of the same type as that of the first arresting construct, or of a different type than that of the first arresting construct.

Optionally, the present monomer units may include a spacer, for example a spacer disposed between the first reporter moiety and the second reporter moiety of the adjacent monomer unit. Nonlimiting examples of spacers are provided elsewhere herein.

Regardless of whether spacer(s) are included in a given example, it will be appreciated that in various examples herein the reporter moiet(ies) may be spaced apart from one another by a sufficient distance that when a given arresting construct pauses translocation of a corresponding reporter moiety through a nanopore, signal from only a single reporter moiety is measured. As such, the monomer unit (e.g., cyclic loop) may be configured such that the first reporter moiety and the second reporter moiety are spaced apart from one another by a minimum distance such that deletion (skip) errors are minimized, as well as the possibility of “read interference” between adjacent/successive reporters.

Optionally, any of the present polymers further may include a first steric lock coupled to the first end of the polymer, and a second steric lock coupled to the second end of the polymer. The first steric lock may be sufficiently large as not to be able to pass through the nanopore or feature thereof (e.g., through constriction 114), thus retaining that end on the first side of the nanopore. The second steric lock may be sufficiently large as not to be able to pass through constriction 114 (such as an oligonucleotide hybridized to polymer 110), thus retaining the second end of the polymer on the second side of the nanopore. As such, regardless of the polarity of a bias voltage that circuitry may apply across the nanopore (e.g., during operations such as described herein) the polymer may remain associated with the nanopore during such operations.

In some examples, compositions are provided herein that include a nanopore having a first side, a second side, and an aperture extending through the first and second sides; and any of the polymers described herein, wherein a first end of the polymer is on the first side of the nanopore, and a second end of the polymer is on the second side of the nanopore. In some examples, devices are provided herein that include such a composition, and circuitry configured to implement any of the methods described herein (e.g., with reference to FIG. 21).

Some examples herein provide a nucleotide that includes a sugar; a nucleobase coupled to the sugar; an alpha phosphate group coupled to the sugar; a first reporter moiety coupled to the nucleobase; a second reporter moiety coupled the alpha phosphate group; and a first arresting construct. Some examples optionally include a second arresting construct. Nonlimiting examples of such monomer units are the F-2-5, F-2-4, F-2-8, F-3-4, F-4-3, F-4-4, F-5-3, F-5-5, B-6-3, and B-6-4 EC codes described with reference to Table 1 and FIGS. 5, 6B, 7, 8A-8B, 9A-9B, 10A-10B, 11, 12, 13A-13C, 14, and 18. The first and second arresting constructs (if provided) may have any suitable location within the nucleotide. For example, the first arresting construct may be disposed between the first reporter moiety and the second reporter moiety. Additionally, or alternatively, for example, the second arresting construct may be either (i) disposed between the first reporter moiety and the sugar or (ii) disposed between the second reporter moiety and the alpha phosphate group.

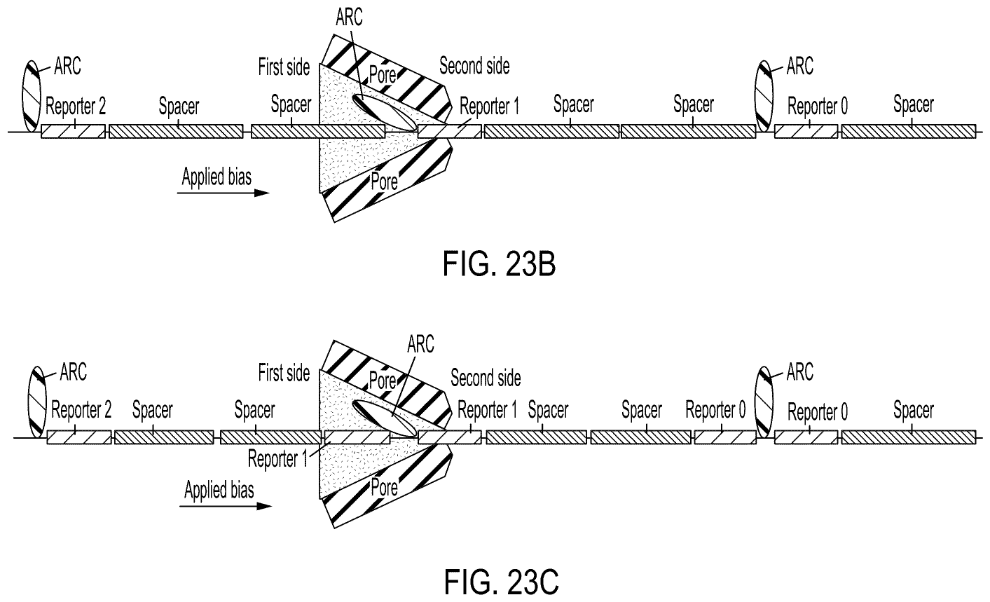

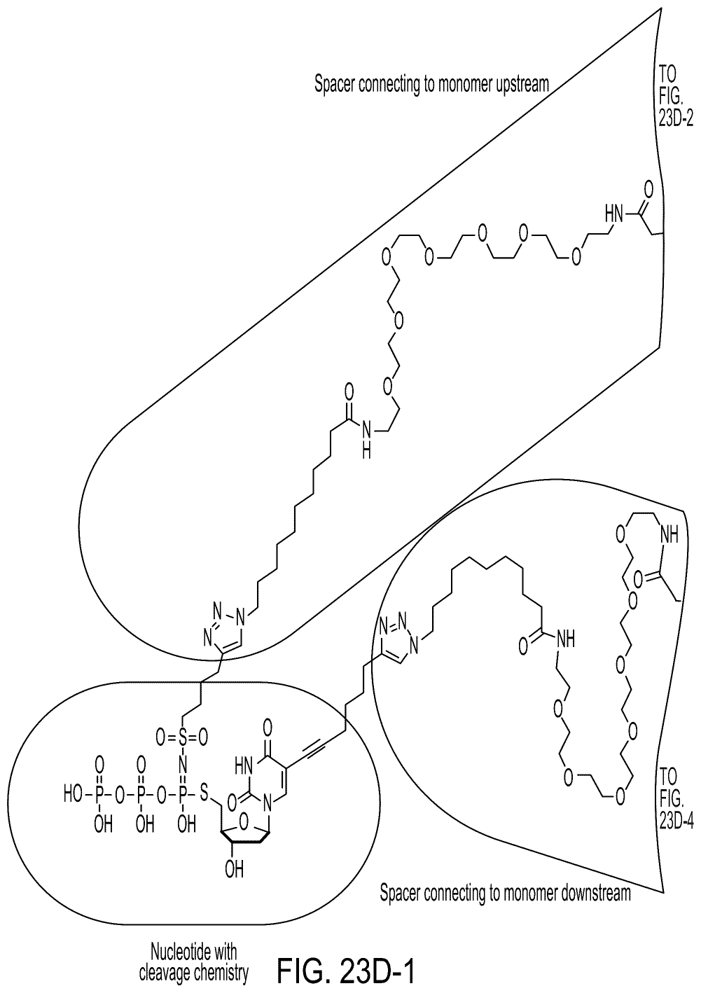

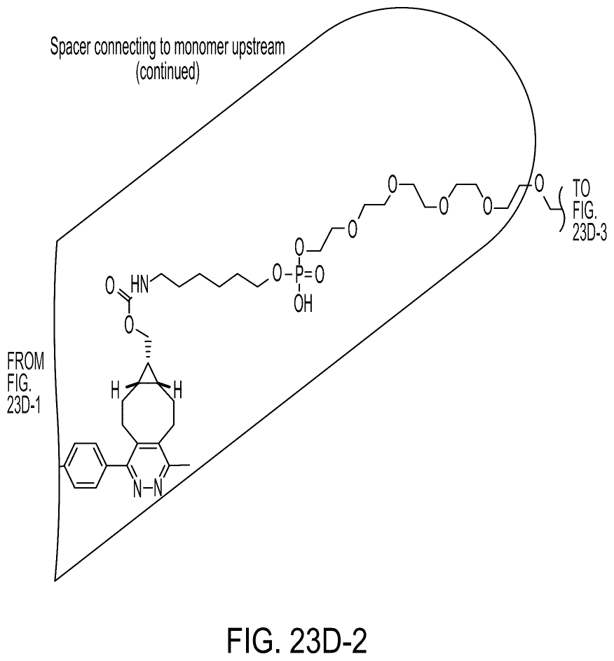

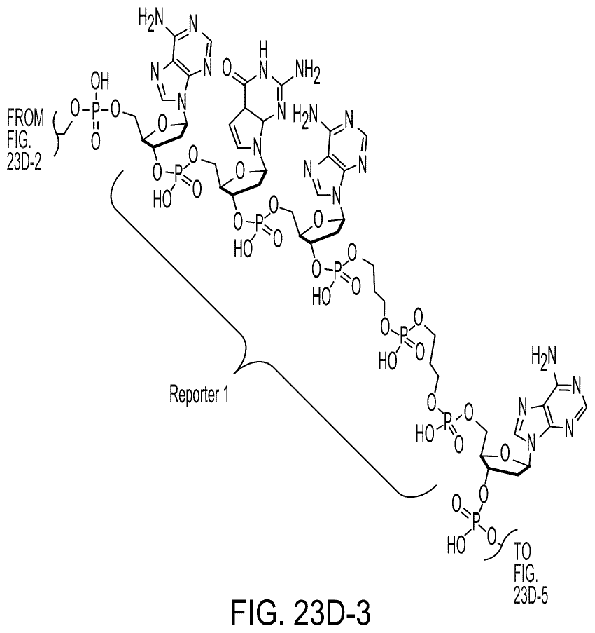

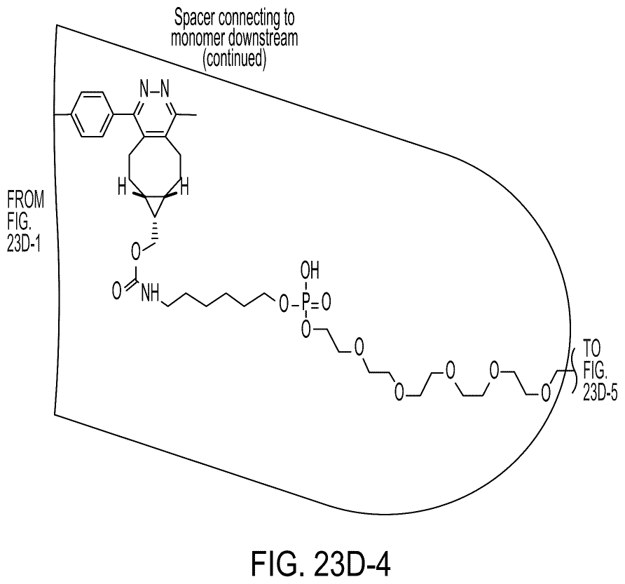

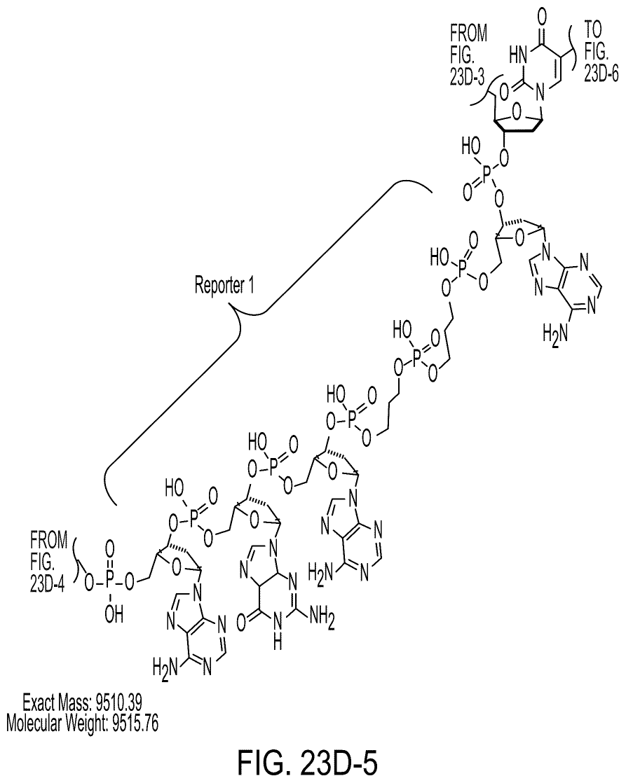

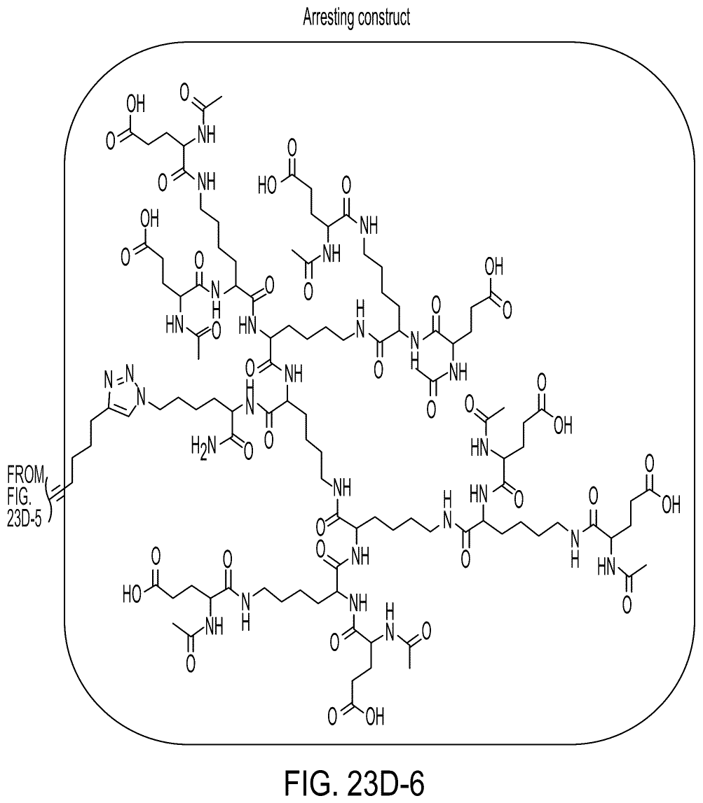

FIG. 23A schematically illustrates an example modified nucleobase with an asymmetric cyclic loop including a single arresting construct and a single reporter moiety. In this example, the reporter moiety (“reporter 1”) is adjacent to the arresting construct (ARC). Additionally, a first spacer is located between the ARC and the nucleobase or alpha phosphate group (collectively referred to as nucleotide+cleavage chemistry), and a second spacer is located between the reporter moiety and the nucleobase or alpha phosphate group. FIG. 23B schematically illustrates a polymer including a monomer unit with the cyclic loop of FIG. 23A, disposed through a nanopore. In this example, under an applied bias between the first side and the second side of the nanopore (“pore”), the ARC pauses the reporter moiety within the aperture of the nanopore, and the reporter is being “read” from the first side to the second side (“forward” method). Alternatively, the reporter may be read from the second side to the first side if the orientation of the reporter region is flipped in the cyclic loop relative to that shown in FIGS. 23A and 23B. Translocation typically proceeds in the direction of reading (e.g., the amount of polymer in the second side increases over time in the “forward” method). In some modes of operation the polymer may be translocated from the second side to the first side while continuing to read from the first side to the second side, e.g., by application of appropriately timed biases.

FIG. 23C schematically illustrates a polymer including a monomer unit with an alternative, symmetric cyclic loop, disposed through a nanopore. The cyclic loop shown in FIG. 23C (monomer unit) includes the same reporter moieties symmetrically disposed on either side of an ARC. Here it may be seen that regardless of the direction of the read (forward or backward) the ARC will pause one of the two reporter moieties in the readhead of the nanopore.

FIGS. 23D-1 through 23D-6 schematically illustrate an example implementation of a modified nucleobase with an symmetric cyclic loop. The cyclic loop shown in FIGS. 23D-1 through 23D-6 (monomer unit) includes the same reporter moieties (FIG. 23D-3 and FIG. 23D-5) symmetrically disposed on either side of an ARC (FIG. 23D-6) labeled as “Reporter 1”. Here it may be seen that regardless of the direction of the read (forward or backward) the ARC will pause one of the two reporter moieties in the readhead of the nanopore. These reporters in this example are composed of nucleosidic reporter moieties and abasic reporter moieties. The spacer (and linker) (FIG. 23D-1, FIG. 23D-2, FIG. 23D-4) are adjacent to the Reporter 1 and attach to the alpha phosphate and nucleobase (labeled as “Nucleotide with cleavage chemistry”).

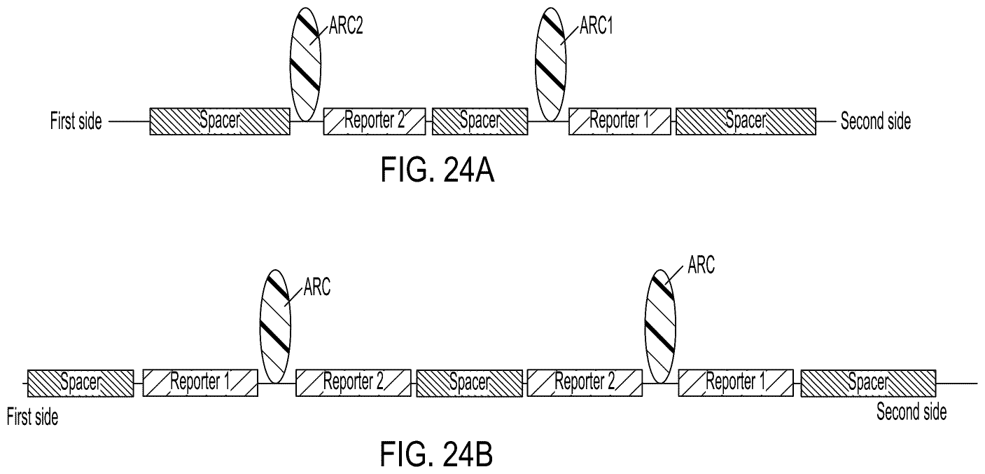

FIG. 24A schematically illustrates a polymer including a monomer unit with an asymmetric cyclic loop including two arresting constructs and two reporter moieties. In this example, the first reporter moiety (“reporter 1”) is adjacent to the first arresting construct (ARC1), and the second reporter moiety (“reporter 2”) is adjacent to the second arresting construct (ARC2). Additionally, a first spacer is located between ARC1 and the nucleobase or alpha phosphate group, a second spacer is located between the second reporter moiety and the nucleobase or alpha phosphate group, and a third spacer is located between the second reporter and the first arresting construct. FIG. 24B schematically illustrates a polymer including an alternative monomer unit with a symmetric cyclic loop including two arresting constructs. In this example, the first arresting construct is adjacent to a first copy of the first reporter moiety (“reporter 1”) and a first copy of the second reporter moiety (“reporter 2”); and the second arresting construct is adjacent to a second copy of the first reporter moiety (“reporter 1”) and a second copy of the second reporter moiety (“reporter 2”). Additionally, a first spacer is located between the first copy of the first reporter moiety and the nucleobase or alpha phosphate group, a second spacer is located between the second copy of the first reporter moiety and the nucleobase or alpha phosphate group, and a third spacer is located between the first copy of the second reporter and the second copy of the second reporter.

Many other options readily may be envisioned on the present teachings.

### ADDITIONAL NOTES

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 nm to about 20 nm should be interpreted to include not only the explicitly recited limits of from about 2 nm to about 20 nm, but also to include individual values, such as about 3.5 nm, about 8 nm, about 18.2 nm, etc., and sub-ranges, such as from about 5 nm to about 10 nm, etc. Furthermore, when “about” and/or “substantially” are/is utilized to describe a value, this is meant to encompass minor variations (e.g., up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods and systems described herein may be embodied in a variety of other forms.

Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Although the foregoing invention has been described in terms of certain preferred examples, other examples will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred examples, but is instead to be defined by reference to the appended claims. All references cited herein are incorporated by reference in their entirety.

The terminology used in the description presented herein is not intended to be interpreted in any limited or restrictive manner and unless otherwise indicated refers to the ordinary meaning as would be understood by one of ordinary skill in the art in view of the specification. Furthermore, examples may comprise, consist of, consist essentially of, several novel features, no single one of which is solely responsible for its desirable attributes or is believed to be essential to practicing the examples herein described. As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this disclosure is in the context of certain examples and examples, those of ordinary skill in the art will understand that the present disclosure extends beyond the specifically disclosed examples to other alternative examples and/or uses of the examples and obvious modifications and equivalents thereof. In addition, while several variations of the examples have been shown and described in detail, other modifications, which are within the scope of this disclosure, will be readily apparent to those of ordinary skill in the art based upon this disclosure. It is also contemplated that various combinations or sub-combinations of the specific features and aspects of the examples may be made and still fall within the scope of the disclosure. It should be understood that various features and aspects of the disclosed examples can be combined with, or substituted for, one another in order to form varying modes or examples of the disclosure. Thus, it is intended that the scope of the present disclosure herein disclosed should not be limited by the particular disclosed examples described above.

1. A method of sequencing a polynucleotide, the method comprising:
(i) disposing a polymer through a nanopore having a first side, a second side, and an aperture extending through the first and second sides, such that a first end of the polymer is on the first side of the nanopore, and a second end of the polymer is on the second side of the nanopore,
wherein the polymer encodes a sequence of a polynucleotide and comprises a sequence of monomer units coupled to one another, each of the monomer units encoding an identity of a nucleotide in the polynucleotide and comprising:
a first reporter moiety;
a second reporter moiety;
a first arresting construct; and
a second arresting construct;
(ii) translocating the first reporter moiety of one of the monomer units into the aperture;
(iii) measuring a first value of an electrical property of the first reporter moiety within the aperture while the first arresting construct pauses translocation of the first reporter moiety;
(iv) translocating the second reporter moiety of the monomer unit into the aperture;
(v) measuring a second value of an electrical property of the second reporter moiety within the aperture while the second arresting construct pauses translocation of the second reporter moiety;
(vi) repeating operations (ii) through (v) for additional monomer units; and
(vii) using the first value and the second value for each of the monomer units to:
identify the nucleotide encoded by that monomer unit; and
distinguish the nucleotide encoded by that monomer unit from the nucleotides respectively encoded by adjacent monomer units, including by any adjacent monomer units that encode the same type of nucleotide as that monomer unit.

2. The method of claim 1, wherein translocating the first reporter moiety of that monomer unit into the aperture comprises applying a first stimulus, and wherein translocating the second reporter moiety of that monomer unit into the aperture comprises applying a second stimulus.

3. The method of claim 2, wherein the second stimulus is applied at a different time than the first stimulus.

4. The method of claim 2, wherein the first stimulus and the second stimulus are of substantially the same magnitude as one another.

5. The method of claim 1, wherein the first reporter moiety is translocated out of the aperture using a constant stimulus, and wherein the second reporter moiety is translocated out of the aperture using the constant stimulus.

6. The method of claim 1, wherein measuring the first value comprises characterizing a first electrical current, ionic current, electrical resistance, or electrical voltage drop across the nanopore while the first reporter moiety is within the aperture; and
wherein measuring the second value comprises characterizing a second electrical current, ionic current, electrical resistance, or electrical voltage drop across the nanopore while the second reporter moiety is within the aperture.

7. The method of claim 1, wherein:
the first reporter moiety uniquely identifies the nucleotide encoded by that monomer unit and
the second reporter moiety does not uniquely identify the nucleotide encoded by that monomer unit; or
the second reporter moiety uniquely identifies the nucleotide encoded by that monomer unit and
the first reporter moiety does not uniquely identify the nucleotide encoded by that monomer unit.

8. The method of claim 1, wherein:
the first reporter moiety uniquely identifies the nucleotide encoded by that monomer unit; and
the second reporter moiety uniquely identifies the nucleotide encoded by that monomer unit.

9. The method of claim 8, wherein for an additional one of the monomer units, a single deletion error causes the first value to be measured and the second value not to be measured, or causes the first value not to be measured and the second value to be measured, the method further comprising:
using the measured first value to uniquely identify the nucleotide and the non-measurement of the second value to identify that the single deletion error occurred; or
using the measured second value to uniquely identify the nucleotide and the non-measurement of the first value to identify that the single deletion error occurred.

10. The method of claim 8, wherein for an additional one of the monomer units, a single insertion error causes the first value to be measured more than once, or causes the second value to be measured more than once, the method further comprising:
using the twice measured first value to uniquely identify the nucleotide and to identify that the single insertion error occurred; or
using the twice measured second value to uniquely identify the nucleotide to identify that the single insertion error occurred.

11. The method of claim 1, wherein:
the first and second reporter moieties, alone, do not uniquely identify the nucleotide encoded by that monomer unit; and
the first and second reporter moieties, together, uniquely identify the nucleotide encoded by that monomer unit.

12. The method of claim 1, wherein:
each of the monomer units further comprises a third reporter moiety and a third arresting construct;
the first, second, and third reporter moieties, alone, do not uniquely identify the nucleotide encoded by that monomer unit; and
the first, second, and third reporter moieties, together, uniquely identify the nucleotide encoded by that monomer unit.

13. The method of claim 1, wherein:
each of the monomer units further comprises a third reporter moiety, a fourth reporter moiety, and a third arresting construct;
the first, second, third, and fourth reporter moieties, alone, do not uniquely identify the nucleotide encoded by that monomer unit; and
the first, second, third, and fourth reporter moieties, together, uniquely identify the nucleotide encoded by that monomer unit.

14. The method of claim 13, each of the monomer units further comprising a fifth reporter moiety, a fourth arresting construct, and a fifth arresting construct,
wherein the fifth reporter moiety does not uniquely identify the nucleotide encoded by that monomer unit.

15. The method of claim 13, further comprising, before (vi) repeating operations (ii) through (v) for additional monomer units:
(viii) translocating the second reporter moiety of that monomer unit out of the aperture while translocating that first reporter moiety into the aperture;
(ix) measuring a third value of an electrical property of that first reporter moiety within the aperture while the second arresting construct pauses translocation of that first reporter moiety; and
(x) using the first, second, and third values together to:
identify the nucleotide encoded by that monomer unit; and
distinguish the nucleotide encoded by that monomer unit from the nucleotides respectively encoded by adjacent monomer units, including by any adjacent monomer units that encode the same type of nucleotide as that monomer unit.

16. The method of claim 1, further comprising, after (vi) repeating operations (ii) through (v) for additional ones of the monomer units:
translocating a plurality of the monomer units to the first side of the nanopore, and then repeating operations (ii) through (vii) to obtain a plurality of additional values characterizing the polynucleotide encoded by the polymer.

17. The method of claim 1, wherein the first arresting construct is disposed between the first reporter moiety and a nucleobase of the monomer unit, or wherein the first arresting construct is disposed between the second reporter moiety and a phosphate group of the monomer unit.

18. The method of claim 1, wherein the second arresting construct is disposed between the first reporter moiety and the second reporter moiety or between the first reporter moiety and the base.

19. The method of claim 1, wherein operations (ii) and (iii) are performed before operations (iv) and (v).

20. The method of claim 1, wherein operations (ii) and (iii) are performed after operations (iv) and (v).

21-80. (canceled)
